(19) United States
(12) Patent Application Publication
(10) Pub. No.: US 2013/0317322 A1
(43) Pub. Date: Nov. 28, 2013

(54) METHOD FOR EVALUATING AND MODIFYING THE STATE OF HYDRATION OF A SUBJECT

(71) Applicant: Audrius Andrijauskas, Vilnius (LT)

(72) Inventor: Audrius Andrijauskas, Vilnius (LT)

(73) Assignee: MEDITASKS, LLC, Tenafly, NJ (US)

(21) Appl. No.: 13/880,946

(22) PCT Filed: Oct. 21, 2011
(86) PCT No.: PCT/US2011/057362

§ 371 Date: Jul. 10, 2013

Related U.S. Application Data
(60) Provisional application No. 61/405,711, filed on Oct. 22, 2010.
(60) Provisional application No. 61/470,224, filed on Mar. 31, 2011.

(51) Int. Cl. A61M 5/172; A61B 5/145; A61B 5/00

(52) U.S. Cl. CPC A61M 5/1723; A61B 5/4875; A61B 5/14546; A61B 5/4839; A61B 5/7278
USPC 600/309; 604/66

(57) ABSTRACT

This invention provides methods and devices for individual evaluating and modifying the state of interstitial hydration of an individual.

This application claims priority of U.S. Provisional Applications Nos. 61/470,224, filed Mar. 31, 2011, and 61/405,711, filed Oct. 22, 2010, the contents of each of which are hereby incorporated by reference in their entirety.

Throughout this application, certain publications are referenced by citation or number in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

1. Field of Endeavor

1.1. Intravenous fluid resuscitation is an integral part of modern medicine practice in a variety of medical fields. Fluid administration aims for optimization of fluid status which is a combination of the optimized circulation and hydration. Administration of intravenous fluids is a common practice during surgery and is indispensable in the management of many nonsurgical medical conditions. It is often a crucial component in areas such as (1) patients undergoing elective, urgent or emergent surgical or obstetrical procedures, (2) patients who elect not to be transfused, (3) patients undergoing treatment in intensive care and toxicology units, (4) critically-ill patients (5) dehydrated patients, and so on.

1.2. The present invention is focused on the administration of fluid for patients with preexisting dehydration or overhydration, and circulating volume deficit (hypovolemia) or overload (hypervolemia), also for patients undergoing surgery and other procedures in which significant fluid and/or blood loss occurs or is expected. This includes but is not limited to: (1) patients undergoing major elective or emergency surgery and obstetrical procedures, major surgery or organ transplantation (2) patients with preexisting disorders of fluid turnover or acquired fluid deficiency states secondary to burns, bleeding or ileus, (3) critically-ill patients.

1.3. The present invention applies to both inpatient and outpatient surgical or hematology settings, and to procedures performed in operating rooms, intensive care units and other locations (e.g., interventional radiology or surgery wards) where fluid replacement is indicated. This new invention is directly applicable to the care administered by anesthesiologists, intensive care doctors, surgeons and individuals who deliver care under their medical direction or supervision.

1.4. The present invention relates to the mathematical model for individual assessment of circulation and plasma hydration, also providing guidelines for optimization and maintenance of the optimized status by means of individualized fluid and transfusion administration.

1.5. The present invention includes a method and apparatus for determining the fluid status from the dynamics of target parameters such as blood hemoglobin concentration during the series of test volume loads of isoosmotic crystalloid solutions. The apparatus can guide administration of fluids and blood transfusion targeted to achieve or maintain the optimal fluid status.

2. Description of the Related Art

2.1. Under the existing processes, fluid administration aims to provide fluid replacement by individually and specifically approaching the body needs for hydration and circulating volume. The main clinical target is to achieve and maintain effective circulating volume in parallel with optimal plasma hydration. Adequate fluid resuscitation is especially important in anemic individuals since transfusion decision is mainly based on the signs of tissue hypoxia in the setting of pre-established normovolemia. Goal-directed fluid therapy or management is the most advanced method for the individual optimization of circulation by intravenous fluid administration. Sophisticated monitoring is required in order to get the most of it. Clinical assessment of hydration status relies on the standardized formulae to calculate preexisting fluid deficits, ongoing losses, and maintenance fluid requirements. Intravascular retention of infused fluid is context-sensitive. With limited clinical applicability, the time course of plasma dilution and the pattern of fluid handling is investigated by volume kinetics. Blood hemoglobin concentration is probably the most frequently obtained blood test in both outpatients and inpatients. It is used for the evaluation of plasma dilution in response to fluid administration.

### Existing Art 1

### Assessment and Optimization of Circulation and Hydration by Goal Directed Fluid Management and Volume Loading Test

2.1.1. Under the existing processes, fluid administration aims to provide fluid replacement by individually and specifically approaching the needs of hydration and circulating volume. The main clinical target is to achieve and maintain effective circulating volume in parallel with optimal hydration. Clinical guidance of fluid management is based on the assessment of circulation and the body fluid balance. Methods ranging from assessment of basic clinical signs to sophisticated monitoring of flow related parameters are used for clinical assessment of circulation. Validated assessment of hydration currently relies on classical nonspecific clinical signs and formulae for estimates of fluid balance.

(a) Under the existing art, as shown in TAB.1, clinical assessment of hydration specifically addresses the calculation of fluid balance and evaluation of nonspecific general signs and symptoms, also conventionally available measures [1]. Textbooks and handbooks recommend a number of standardized formulae to calculate preexisting fluid deficits, ongoing losses, and maintenance fluid requirements [2]. New methods for the more specific and sensitive evaluation of hydration state such as Volume Loading Test (VLT) method, which is disclosed in U.S. 2007/0178167 A1, hereby incorporated by reference in its entirety.

(b) Under the existing art, e.g. U.S. 2007/0178167 A1, the Homeostatic Blood States method or HBS Method proposed an algorithm—the Volume Loading Test (VLT)—that serves for clinical assessment of the state of hydration. It is based on the concept that kinetics of intravenously administered isotonic or nearly isotonic fluids are dependent on the state of hydration, so that more crystalloid fluid is retained in plasma in the setting of preexisting dehydration. An algorithm or steps of VLT-test procedure evaluates absolute changes in blood hematocrit by comparing it at two checkpoints—just before the crystalloid test-volume load (TVL) infusion, and after the steady state without fluids. The rate of infusion is defined as “high rate bolus” that usually takes 10-15 minutes, and the amount recommended is the “author's preferred rate in preliminary investigations” which is 10 ml/kg/h. The steady state after the end of infusion is referred to as equilibration pause lasting for 20 minutes. If Hct decreases for less than 0.01, the preexisting homeostatic target state is assumed. The later is presumably associated with optimal plasma hydration. If Hct decreases for more than 0.01, the preexisting dehydration is assumed, and another test fluid load is indicated. That continues until Hct decreases for less than 0.01. Corrections for presumably osmotically induced changes in hematocrit also apply.

(c) Under the prior art, clinical assessment of circulating volume is indirect and referred to as assessment of circulation or fluid status or volume status or volemia. It is based on a range of clinical signs and flow related parameters. The blood flow delivered by the heart, or the total mean flow in the circulation, is referred to as cardiac output. The output during a single heartbeat is the cardiac stroke volume (SV). The systemic peripheral resistance affecting the blood flow depends on the geometry of blood vessels (the inner radius and its length) and viscosity of blood.

Arterial blood pressure is a critical hemodynamic parameter. Noninvasively it can be measured indirectly by means of sphygmomanometer. Invasive methods require catheterization of arterial blood vessels. The pressure in the left heart chambers is obtained only invasively by inserting catheters into brachial or femoral arteries. Catheters inserted into an antecubital vein and advanced until they reach small branches of the pulmonary artery record the pulmonary artery wedge pressure—the pressure downstream from the catheter tip, that is, the left atrial pressure. Clinical measurements of venous pressure are typically made by inserting a catheter into the jugular or subclavian vein. In the research laboratory, capillary pressure can be obtained by means of the inserted micropipette.

The spectrum of blood flow measurements in the circulation ranges from determinations of systemic blood flow to assessment of flow within an organ or a particular tissue within an organ. The most frequently used modern invasive instruments for measuring blood flow are electromagnetic and ultrasound flow meters based on Doppler effect. In clinical practice, noninvasive ultrasound methods are widely used. Plethysmographic methods are noninvasive approaches for measuring blood flow of a limb, or even a whole person. Cardiac output can be measured indirectly by the Fick method, which is based on the law of conservation of mass (measuring rate of uptake or elimination of a substance by an organ). The dye-dilution method is a variation of the Fick procedure (the simultaneous downstream measurement of the injected substance). Regional blood flow can be measured by clearance methods, which are another application of the Fick principle.

(d) Under the existing art, the reach for predefined hemodynamic values and the fixed volume fluid administration regimens referred to as restrictive and liberal strategies [FIG. 1] are currently being replaced by individual and context-sensitive approaches such as goal directed fluid management. It optimizes of fluid status by individual maximization of the flow-related parameters referred to as target parameters [3]. The GDM strategy was introduced in 2002 [4]. In contrast to the previously dominating fixed volume regimens and the reach for predefined hemodynamic values, individual features are taken into account [FIG. 2].

(e) Under the existing art, a range of static and dynamic flow related parameters [FIG. 3] are used for the evaluation of fluid status and fluid responsiveness in goal directed fluid management. Static parameters specifically reflect the fluid status, while dynamic measures address the fluid responsiveness which is a prediction of cardiovascular response to the fluid load before it is administered. Evaluation of fluid status by means of most reliable static and dynamic parameters [FIG. 4] is described in the GDM algorithm, part A [5].

In the recent past, central venous pressure and pulmonary artery occlusion pressure were used for guiding fluid therapy. Later, the volumetric measures of cardiac preload such as global end diastolic volume index were found to be much more fluid-state-specific than cardiac filling pressures [6]. Cardiac output and especially stroke volume have been reported to be the more reliable. They became a standard of care in selected patients, especially when derived by minimally invasive validated methods such as esophageal Doppler [7].

Currently, to the best knowledge of the inventor, the cardiac stroke volume is widely acknowledged as being the most specific circulating volume target parameter used for goal directed fluid management. Meanwhile cardiac output is not that specific due to its dependency on the heart rate. Optimized fluid status is assumed when maximized cardiac stroke volume is reached. Such cardiac performance is associated with maximal contractility of the myocardium. That functional state is referred to as the highest point on the Frank-Starling curve of the heart [FIG. 5]. It was shown to improve functional parameters, also reduce hospital stay and morbidity after major surgery [8-11]. Unlike static parameters, dynamic parameters are poor markers of fluid status, but they specifically reflect the fluid responsiveness, which is a prediction of cardiovascular response to volume expansion and related increase in cardiac preload induced by fluid loading [FIG. 5]. Stroke volume variation and pulse pressure variation were reported to be reliable in evaluation of fluid responsiveness in cardiac surgery, cardiac failure and sepsis [12], and also in animals subjected to graded hemorrhage [13]. Conclusion regarding the preexisting fluid status can usually be drawn from the evaluation of static and dynamic flow related parameters. Hypovolemia, normovolemia or hypervolaemia may be suspected [FIG. 4]. The strength of evidence in the support of these conclusions is described in FIG. 6.

The strongest evidence is provided by the combination of consistent findings derived from basic clinical evaluation, volumetric measures of preload and dynamic flow related parameters, especially those defining stroke volume responsiveness. The same criteria and parameters can be used for the evaluation of actual response to the fluid loading [FIG. 7] as described in the GDM algorithm, part B [5]. Under the existing art, actual response of the target parameter is evaluated after the 5 min steady state following 200-250 ml fluid challenge of colloid. A 10% increase in SV is referred to as “responder” case, also justifying further fluid challenge. Subsequent intravenous fluid loads are repeated until SV no longer increases (“non-responder” case). The later is assumed as indicating that maximization of SV is complete and the ‘flat part’ of the Starling curve was reached [14].

Detailed guidelines for the maintenance fluid administration and optimization of oxygen transport are disclosed in textbooks of anesthesiology and intensive care, also surgery and other specialties dealing with intravenous fluid administration [15]. Simplified approach was proposed by the inventor in part C of GDM algorithm [FIG. 8] for peri-operative fluid management and hemodynamic monitoring in non-cardiac surgery patients [5].

Optimization of fluid status and maximization of stroke volume induced by fluid loading may not be associated with normalization of arterial blood pressure, which is an important clinical endpoint. In such cases, associated increase in stroke volume and ABP may be achieved by administration of inotropic and sympatomimetic drugs [FIGS. 8,9]. In case of co-existing anemia, transfusion of packed red blood cells (PRBC) may provide the similar effect. In summary, cardiac stroke volume and its variations are the most specific target parameters in goal directed fluid management which is associated with improved outcomes in critically ill and those undergoing major surgery (11).

(f) Under the existing art, a range of methods and devices for the measurement of stroke volume and its variations are used in research and clinical practice. Traditional invasive methods are currently being replaced by minimally or non-invasive methods. The minimally invasive method for measuring stroke volume SV and SVV is based on arterial pulse contour analysis [16]. The calibrated measurements are based on the Fick principle and indicator dilution methods. That method is deployed by techniques such as LiDCO™Plus (LiDCO™ Cambridge, UK), and provides the most precise pulse contour analysis derived static and dynamic parameters. The pulse contour analysis may not need calibration of the monitoring device. For example, the PiCCO™ technique (PULSION Medical Systems, CA, USA) does not need calibration, but requires central venous and arterial access [17]. The minimally invasive technique is used by LiDCO™Rapid (LiDCO™, Cambridge, UK) and FloTrac™ Nigileo™ (Edwards Lifesciences, USA, version 1.07), since it requires catheterization of a single and even small artery [16]. Similar to LiDCO™Rapid, it can determine cardiac output, stroke volume, and SVV via existing catheter inserted in the radial artery as part of the standard monitoring.

Monitoring of stroke volume became a standard of care in selected patients thanks to the minimally invasive validated methods such as esophageal Doppler (CardioQ™, Deltex Medical, USA) [7]. Fluid management guided by esophageal Doppler derived SV and SVV has been reported to improve peri-operative outcome (11). Bio-impedance based technique such as PhysioFlow™ is a non-invasive option for measuring cardiac output. The method is validated against generally accepted reference methods, such as “direct” Fick, thermodilution and Echo-Doppler at rest and during exercise, on healthy subjects and patients suffering from cardiac or pulmonary diseases[18].

Software applications are also used for deriving flow related cardiac parameters. New technology such as HEARTSMART® (APC Cardiovascular Ltd, Crewe, UK) uses empirical physiological algorithms in their computer software programs. The static parameters are continuously calculated in real time by deploying the standard physiological variables such as central venous pressure, heart rate, mean arterial pressure, body height and weight, temperature and age. Another dynamic flow related parameter is the pleth variability index. It is used to monitor the respiratory variations in the pulse oximeter plethysmographic waveform amplitude. The PVI® is part of the upgradable Masimo Rainbow SET® platform, which is another technology for noninvasive assessment of fluid responsiveness. Zimmerman et al. reported that PVI is useful for the assessment of fluid responsiveness in mechanically ventilated patients undergoing major surgery, and its accuracy is comparable with the measurements of stroke volume variation (European Journal of Anaesthesiology 2010; Vol 27.)

### Below Follow the Deficiencies Related to Existing Art 1 [2.1.1]

2.1.2. However, systematic reviews of randomized clinical studies found no evidence to recommend one type of fluid therapy over another or provide guidance on the rational fluid administration applicable in most clinical settings [8]. Individual assessment of hydration and volume status remains disputable and challenging for decades. Inadequate intravenous fluid administration is an ongoing clinical concern. Thus, there is an obvious need for a relatively simple and reliable bedside method of estimating cardiac function. As a result, there is a continuing search for a method of flow related measurement that is more accurate and less invasive than its predecessors.

That is because:

(a) Clinical assessment of hydration relies on nonspecific signs and symptoms. In clinical practice, standardized formulae from the textbooks for the calculation of preexisting fluid deficit, ongoing losses and maintenance fluid requirements serve as rules-of-thumb, and they are modified into a range of strategies and guidelines for perioperative fluid administration [2].

(b) The art-described by the Volume Loading Test (VLT-test) for the assessment and optimization of hydration undergoes validation. Aside for that fact, it is time consuming, does not account for the dynamics of arteriovenous difference in plasma dilution and considers absolute difference in pre- and post-infusion hematocrit aiming to discriminate different states of hydration. Also, the suggested test volume loads may be excessive especially in preexisting states of over hydration and hypervolemia.

(c) Conventionally available circulation related measures are the arterial and venous blood pressures. However, they are non-specific and clinically unreliable since the relationship between the filling pressure and volume cannot be determined clinically [6]. Although some more specific and sensitive flow related parameters are routinely measured in critically ill and in high-risk patients, obtaining them is associated with inherent risk and cost of related procedures [5,14]. Traditionally used static parameters of preload were shown to be poor markers of volume status since the relationship between cardiac filling pressure and filling volume cannot be determined clinically. Instead, they may be associated with inherent risk that overcomes the clinical benefits, and these problems are well described. For example, the pulmonary artery catheter technique was consistently reported to be overly invasive for the information that it provides.

(d) The reach for predefined hemodynamic values and the fixed volume fluid administration regimens referred to as restrictive and liberal strategies were dominating for years, but all of them have failed to demonstrate absolute advantages or propose a gold standard for the fluid resuscitation.

(e) Despite accumulating evidence that goal directed fluid management is associated with better outcomes in surgical population and critically ill, there are serious deficiencies and limitations in its clinical applicability and data interpretation. Aside from limitations in clinical applicability of preload measurements such as assessment of global end diastolic volume index, data interpretation is clinically challenging. For example, intermediate values may be associated with different volume status: moderate hypovolaemia, normovolaemia, or moderate hypervolaemia. Thus, even the most reliable volumetric parameter of preload is an insensitive indicator of volume status. It remains relatively unchanged despite reduced blood flow to certain organs such as the gut. Meanwhile, occult hypovolaemia leading to poor organ perfusion is thought to be a major factor in determining postoperative morbidity after major surgery. In summary, evaluation of preload is currently limited to cases when it is a standard of care [6]. The routine estimation of most specific and reliable flow-related parameters such as SV and its variation is also uncommon, mainly because even minimally or non-invasive methods are cumbersome, expensive and potentially inaccurate. When it comes to the predictive parameters of fluid responsiveness, even the need for a synus rhythm and mechanical ventilation of lungs is a serious limitation. Despite the reports demonstrating their accuracy they still need validation, and especially in the dynamically changing settings such as acute hemorrhage. Moreover, the they are subject to inherent errors inherent for the method of obtaining and processing the readings. For example, Lahner (Br J Anaesth 2009; 103:346-351.) recently reported that accuracy of SV variations determined by arterial pulse contour analysis was not reliable for the evaluation of fluid responsiveness in major abdominal surgery patients. Serious deficiencies arise from the following features inherent to the dynamic parameters of preload: (a) parameters non-specifically reflect the ability of preload to increase in response to an increase of intrathoracic pressure, and (b) specifically reflect the ability of SV to increase in response to an increase of preload. Assumption is made that, most likely, similar response of preload and SV will be induced by the fluid challenge. However, it is not clear whether (a) preload is responsive, and (b) fluid loading will induce volume expansion sufficient to significantly increase preload and consequently SV. More specifically, dynamic parameters ≧10% are associated with potentially responsive SV by predicting its significant increase (≧10%) under the condition that preload is adequately increased by the fluid load. However, the latter condition is hard to predict, since it is dependent on several interfering factors such as plasma volume expansion efficacy of infused fluids and responsiveness of preload, the latter also being dependent on extrinsic factors such as intrathoracic pressure, etc. Note that plasma volume expansion efficacy of the fluid load is dependent on numerous factors other than inherent characteristics of the infused fluid. Meantime, alternative predictors of fluid responsiveness, such as amplitude of plethysmographic signal or the passive leg raising test still need validation. Optimization of the fluid-status in the goal directed fluid management is usually based on evaluation of actual SV response to the fluid challenge. However the method is hampered by the striking variability of responses reported in literature [13]. Hadian et al. also found that only 40% to 72% of intensive care patients with hemodynamic instability responded to fluid load by a significant increase in SV and/or cardiac output (Curr Opin Crit Care. 2007; 13, 318-323). Holte et al. proposed explanation that from the angle of pathophysiology introducing the context-sensitive pattern of fluid handling. It claims that fluid requirements and handling of infused fluids vary according to individual physiology and specific circumstances such as anesthesia and surgery (Br J Anaesth 2002; 89, 622-632.). Thus, variability in SV response may be caused by one or more factors, such as (a) low plasma volume expanding efficacy of the infused fluid (e.g., capillary leak or bleeding), (b) nonresponsive preload (e.g., venous pooling or high intrathoracic pressure), (c) nonresponsive stroke volume (e.g., preexisting maximized myocardial contractility such as the flat part of the Frank-Starling curve, due to fluid overload and/or a failing heart). An increase in blood volume is crucial to initiate the fluid load induced “chain reaction” (circulating volume→preload→SV), potentially increasing SV at the end point. Thus, without assessment of blood-volume expansion efficacy of the fluid load and responsiveness of both the preload and SV, the actual SV response to the fluid challenge may be misinterpreted. However, just like other methods for the evaluation of cardiovascular response to fluid loading, the algorithms for goal directed fluid management lack evaluation of actual plasma dilution induced by the volume challenge. Meanwhile, determining the actual plasma volume expanding efficacy of the infused fluid in association with actual response of flow related parameters could facilitate the better and wider clinical interpretation of fluid responsiveness and/or actual response. However, as discussed in the previous chapters, under existing state-of-the-art, evaluation of actual plasma volume expansion induced by the fluid load is missing in the conventional clinical evaluation of cardiovascular response to the fluid challenge.

(f) Due to limited on-site applicability and availability, also the cost and risk versus benefit ratio and lack of accuracy the monitoring of flow related parameters is limited to cases when it is a standard of care. Thus, currently it is available in the minority of clinical settings. Although traditional invasive methods are currently being replaced by minimally or non-invasive methods, they all lack validation.

### Existing Art 2

### Volemia

### Volume Status

2.1.3. Body hydration status dependent volume of free water in plasma is part of the circulating blood volume. Under the existing processes, in the context of fluid management, the physiology of circulation is approached from the angle of adequacy of central blood volume or volemia. Understanding the complexity of association between the central blood volume and the total blood volume, between plasma hydration and plasma dilution, also the principles of blood flow distribution is vital for the rational fluid management.

(a) Under the prior art, in human physiology, the distribution of blood flow is described as blood flow from the heart to arteries, which branch and narrow into arterioles, and then branch further into the capillaries [19]. After the tissue has been perfused, capillaries join and widen to become venules and then widen more to become veins, which return blood to the heart [FIG. 10]. Arteries are considered as distribution system [19], microcirculation—as diffusion and filtration system and veins—as a collection system [20]. The body's total blood volume is not uniformly distributed. There are several approaches in grouping the blood volumes [19]. The ˜85% of it resides in the systemic circulation, the ˜10% in the pulmonary and ˜5% in the heart chambers. The ˜15% of it resides in the high pressure system, the ˜80% of it resides in the low pressure system and ˜5% in the heart chambers. Most of the blood volume resides in the systemic veins. From the 85% of the total blood volume that resides in the systemic circulation, approximately ¾ (65%) is on the venous side, particularly in the small veins. The fourth approach to grouping blood volumes is dividing into central blood volume (volume of the right heart and pulmonary vessels) and peripheral, which is the rest of the circulation [19]. The central blood volume is very adjustable and constitutes the filling reservoir for the left heart. The distribution of blood flow governs the distribution of blood volume within the body according to the flow-pressure-volume relationship [21].

(b) Under the prior art, the simplistic definition of normovolemia is applied to the state of circulation that provides sufficient perfusion of vital organs and tissues. Mark et al. notices a variety of definitions for normovolemia [22]. For example, Smith defines nrmovolemia as normal blood volume of healthy individuals, and assumes it being ˜75 ml (kg body weight)⁻¹ (Smith and Kampine, 1999). According to Schrier, the effective circulating blood volume refers to the part of the volume within the arterial system effectively perfusing the tissues (Schrier, 1990; Abraham and Schrier, 1994), and it is regulated by the interplay between the circulatory system and the kidneys according to Guyton (Guyton et al. 1980). Changes in the venomotor tone can compensate for variations in the effective circulating blood volume. The effective circulating blood volume is assumed to depend mainly on the central blood volume, that is, the blood available to the heart [22]. A functional definition of ‘normovolemia’ is the ability to provide the heart with an appropriate central blood volume, i.e. cardiac preload (Ejlersen et al. 1995; Jenstrup et al. 1995). Hypovolemia may be characterized by a reduced preload to the heart, i.e. with stroke volume becoming dependent on central blood volume [22]. The reported increase in CO with volume loading is taken to imply that a patient is preload responsive (Pinsky, 2002; Boulain et al. 2002). Conversely, the intravascular volume may be expanded beyond the volume that can provide ‘maximal’ CO at rest. By interpolation between hypovolemia and hypervolemia, the state of normovolemia may be considered as the point in the cardiac preload and output relationship at which cardiac output does not increase or decrease under circumstances where venous return is unimpeded [22]. It is clear from the above that normal blood volume in the sense of the volume calculated by current formulae for normal blood volume does not warrant normovolemia and adequate perfusion of every vital capillary bed.

(c) Under the existing art, the venous system has a specific and important role in providing the heart with appropriate central blood volume. The main functions of the venous system are to return blood to the heart from the periphery and to serve as a capacitance to maintain filling of the heart. Gelman has recently addressed some confusion in the literature regarding the definitions of venous compliance and capacity, also providing proper clarification that follows [21]. Venous capacity is a blood volume contained in a vein at a specific distending pressure. Venous compliance is a change in volume of blood within a vein (or venous system) associated with a change in intravenous distending pressure. Therefore, capacity is a point of volume at a certain pressure, while compliance is a slope of change in volume associated with a change in pressure. A decrease in volume within a vein (or venous system) can be achieved by a decrease in capacity (position of the curve) or by a change in compliance (slope of the curve) or both. Change in blood volume within the veins is associated with relatively small changes in venous pressure since the veins contain approximately 70% of total blood volume and are 30 times more compliant than arteries [21]. The splanchnic system receives approximately 25% of cardiac output and contains approximately 20% of total blood volume. Because of high compliance of the veins, changes in blood volume are associated with relatively small changes in venous transmural pressure [21]. Veins are the most compliant vasculature in the human body and are easily able to accommodate changes in the blood volume. Therefore, they are called capacitance vessels and serve as a reservoir of blood that easily and immediately changes volume in it to maintain filling pressure in the right heart. Splanchnic and cutaneous veins are the most compliant and represent the largest blood volume reservoirs in the human body [21]. Veins of the extremities are less compliant than splanchnic veins, and therefore, their role as blood volume reservoir is relatively minimal. Gelman clarifies that the relation between flow, pressure, and volume within the veins occurs in very compliant (splanchnic) veins and represents a passive distribution of volume between veins (mainly the splanchnic system) and the heart, which is associated with changes in venous capacity without change in compliance [21]. A decrease in flow through the splanchnic arteries, being associated with a decrease in volume in the splanchnic veins and the liver and transfer of this volume into the systemic circulation, plays an important role not only in compensation of hypovolemia but also in compensation of cardiac failure. If CO decreases, a simultaneous decrease in flow through splanchnic arteries is associated with a shift of blood volume from splanchnic veins to the heart recruiting Frank-Starling mechanism (an increase in preload leading to an increase in contractility) [21]. Constriction of the veins decreases their capacity and expels blood from them into the systemic circulation. However, venoconstriction may increase venous resistance and subsequently decrease venous return and CO. How is the body recruiting the blood volume without an increase in resistance to venous return? The constriction of splanchnic veins is not associated with an increase in resistance to venous return because the splanchnic system is outside of the mainstream of blood flow to the heart through the caval veins [23]. That explains why patients at the beginning of bleeding (up to 10-12% of blood volume loss) maintain their systemic hemodynamics well without changes in heart rate, blood pressure, or central venous, yet later, hemodynamics quickly deteriorates. However, when the entire venous depot of blood has been mobilized, decompensation occurs suddenly. Similarly, when mobilization of volume is secondary to an increase in sympathetic tone, an intervention associated with venodilation (general or regional anesthesia, opioids, sedatives), may cause rapid decompensation without additional blood loss because of venodilation [21]. Changes in resistance in the small arteries and arterioles may affect venous return in opposite directions. In physiology textbooks, such processes as integrated response to hemorrhage are explained in a relatively complex way [FIG. 11] [19]. The simplistic but very practical for clinicians explanation was proposed by Gelman, who explained these processes by a two-compartment model of venous compartment: compliant (mainly splanchnic veins) and noncompliant (nonsplanchnic veins) [FIG. 12] [21]. Even the similarity of venous compartments to the water in the tub was used for the further explanation [FIG. 13].

(d) Under the prior art, turning a fluid load into a significant increase of cardiac stroke volume solely by the intravenous fluid infusion requires significant increase in blood volume which in turn would increase the venous flow to the heart (cardiac preload). Acknowledging the inherent role of blood volume in the interaction among cardiovascular subsystems and non-circulatory systems [FIG. 14], the concept of physiological ‘chain reaction’ was proposed by the inventor [5]. According to the physiological ‘chain reaction’, a significant increase of blood volume induced by the fluid load is considered as major condition on the way of turning a fluid load into a significant increase of cardiac function. More specifically, fluid induced plasma dilution is considered as the specifically fluid administration related trigger for the endogenous regulatory accommodations that ideally would lead to significant increase in stroke volume:

blood volume→preload→stroke volume

That approach shows the need for the clinically applicable evaluation of plasma dilution along with or even without parallel assessment of flow related target parameters.

### Below Follow the Deficiencies Related to Existing Art 2[2.1.3]

2.1.4. However, assessment of volemia remains clinically challenging for decades.

That is because:

(a) As recently noticed by Mark et al. [22], despite the advanced understanding of underlying processes and sophisticated monitoring modalities, there is a variety of definitions for normovolemia. The simplistic definition of normovolemia is applied to the state of circulation that provides sufficient perfusion of vital organs and tissues. Meanwhile, a functional definition of ‘normovolemia’ is defined as the ability to provide the heart with an appropriate central blood volume, i.e. cardiac preload (Ejlersen et al. 1995; Jenstrup et al. 1995). However, the usual clinical and haemodynamic parameters are not reliable indices of preload to the heart (Boulain et al. 2002; Pinsky, 2002) and an ‘optimal’ volume is neither defined nor is it an easily measurable entity. Moreover, there is no consensus regarding the clinical definitions of “volume status”, “fluid status”, “hydration status”, “plasma dilution” and “plasma hydration”. Very frequently, in literature and clinical practice, the plasma dehydration is referred to as hypovolemia, and over hydration—as hypervolaemia. That is misleading in many cases since, in order to cause hypovolemia, the dehydration of plasma has to be severe and present in the setting of acute anemia in order to be associated with uncompensated absolute decrease of circulating blood volume. Moreover, in conventional clinical practice, the low blood hematocrit is associated with a “diluted” individual, and high blood hematocrit is associated with a “concentrated” individual, while it can be on the opposite. In fact, we still cannot discriminate between dehydration, normal hydration and over hydration in association with any state of volemia.

(b) Interaction between flow, pressure, and volume within the veins occurs in very compliant (splanchnic) veins and represents a passive distribution of volume between veins (mainly the splanchnic system) and the heart, which is associated with changes in venous capacity without change in compliance. That is probably the main reason why it is clinically impossible to estimate without significant margin of error the blood volume reserve in the splanchnic and cutaneous veins that are extremely compliant and represent the largest blood volume reservoirs in the human body. The change in venomotor tone is clinically very important since it can compensate for variations in the effective circulating blood volume. However, the venomotor tone cannot be directly monitored in clinical settings.

(c) Acknowledging that turning a fluid load into a significant increase of cardiac stroke volume is the major task of the fluid management, there is a lack of clinically applicable evaluation of plasma dilution. Being a reflection of dilution origin changes in blood volume, monitoring of plasma dilution would be most useful if applied along with parallel assessment of flow related target parameters.

### Existing Art 3

### Interaction Between Blood Volume and Fluid Turnover

2.1.5. Under the existing processes, the blood volume is a variable with inherent contribution to the function of circulation and perfusion of tissues [19]. Normal volume is traditionally associated with normovolemia. Free water or hydrating fluid in plasma is part of the blood volume. Its amount in blood is changing in accordance with multiple processes and especially the body fluid turnover—intake, distribution and elimination. Since intravenous fluid administration is not a physiological way of fluid intake, monitoring of related blood volume changes and distribution of infused fluid is especially important for the rational fluid resuscitation.

(a) Under the prior art, the most accurate blood volume is obtained by the direct measurements, especially simultaneous measurements of red cell mass and plasma volume.

(b) Under the existing art, a range of mathematical methods are used to calculate the normal blood volume. For example, the FDA approved 1271 DE Guidance Document Section IV [24] recommends calculating blood and plasma volumes for donors in the 45-100 kg range as follows:
The blood volume in milliliters may be determined by dividing the body weight in kilograms (kg) by 0.015, or alternatively by multiplying the body weight in kilograms by 70 ml/kg.
The plasma volume in milliliters may be determined by dividing the body weight in kilograms (kg) by 0.025, or alternatively by multiplying the body weight in kilograms by 40 ml/kg.

(c) Under the prior art, the normal blood volume as being the homeostatic target that the body strives to maintain has been recently questioned. The concept of physiologic target blood volume was proposed by the volume kinetics [25-26] and homeostatic blood states theory [27-28]. The later is disclosed in part by the US patent pending Homeostatic Blood States method or HBS Method. The most powerful concept of the two theories is the mathematical demonstration of a physiologic or homeostatic target blood volume that intravascular volume will approach, usually quite rapidly after perturbation following the intravascular fluid load. Both theories proposed a mathematical way of estimating the target volume. Volume kinetics estimates it from the trends of plasma dilution induced by the iv test volume load. Meanwhile, the HBS method's estimates are based on the invented formulae that deploy blood hematocrit value, which is obtained in the state of optimised plasmadilution. Moreover, the HBS Method proposed a volume loading test (VLT) method for achieving the presumed state of optimal plasmadilution.

(d) Under the prior art, the body strives to maintain the physiologic target volume of fluid spaces by distributing fluid over central and peripheral functional fluid spaces. Similarly, the homeostatic blood states theory [27-28] claims that the homeostasis of the human body strives to maintain the red cell mass specific homeostatic target blood volume. It is equal to ideal (normal) blood volume and maintains ideal plasma volume only once per physiologic hematocrit scale—at hematocrit of Ideal Total Match which is most likely equal to Hct-0.40 (a state consistent with ideal rheology of blood). Intracellular fluid comprises two thirds of the body water. The remaining one third, approximately 15 l in the normal adult, designates the extracellular volume, consisting of the plasma (approximately 3 l), the interstitial space (approximately 12 l), and small amounts of so-called transcellular fluids, such as gastrointestinal secretion, cerebrospinal fluid, and ocular fluid [29]. In human physiology, blood flows from the heart to arteries, which branch and narrow into arterioles, and then branch further still into the capillaries [19]. After the tissue has been perfused, capillaries join and widen to become venulae and then widen more to become veins, which return blood to the heart [FIGS. 10,15]. The plasma is a carrier of the fluids that are used by the processes of hydration. Transcapillary exchange of that fluid is related to multiple factors such as hydraulic pressures, distribution of osmotic active substances, function of the biologic barriers and oxygen-consuming ion pumps. The intact vascular barrier cannot be crossed by large molecules and proteins in relevant amounts [30]. This is important because it enables the circulation to generate a positive intravascular blood pressure without unlimited fluid loss toward the interstitial space. Ernest Starling, a British physiologist, introduced the classic model of the vascular barrier as early as 1896 claiming that inside the vessels, the hydrostatic pressure is high, as is the colloid osmotic pressure [FIGS. 16,17] [31]. In continuous capillaries where permeability for water and small solutes is high and permeability for proteins is low, transvascular fluid exchange is conventionally described by the Starling formula:

Jᵥ/S=Lₚ(ΔP−σΔΠ)

where Jᵥ—filtration rate per unit area, Lₚ—hydraulic permeability of the vessel wall (fluid conductivity), ΔP—hydrostatic pressure difference between capillary lumen and tissue, σ—refection coefficient to the plasma proteins, ΔΠ oncotic pressure difference between capillary lumen and tissue.

A healthy vascular endothelium [FIG. 18] is coated by the endothelial glycocalyx [32-34]. This structure is a layer of membrane-bound proteoglycans and glycoproteins. Recently, the endothelial glycocalyx bound plasma proteins and fluids have been identified [35]. This layer, together with the endothelial cells, is part of the double-barrier concept of vascular permeability, identifying the glycocalyx as a second competent barrier in addition to the endothelial cell line opposing to unlimited extravasation. By exerting a vital role on the physiologic endothelial permeability barrier [32] and preventing leukocyte and platelet adhesion, it mitigates inflammation and tissue edema [36]. The amount of plasma fixed within the endothelial surface layer and, therefore, quantitatively not participating in the normal blood circulation is approximately 700-1,000 ml in humans [35]. However, this noncirculating part of plasma volume is in a dynamic equilibrium with the circulating part [37]. The prerequisite for the classic Starling principle to be able to bind water within the vascular system is a significant colloid osmotic pressure gradient between the intravascular and extravascular space. However, several experiments have shown that this equation cannot be correct [38]. The expected lymph flow, based on calculations according to the Starling principle, does not equal the measured flow [39]. Even after equilibration of intravascular and extravascular oncotic pressure in the isolated single microvessel model, the vascular barrier function remains intact [33]. There seems to be an oncotic gradient directly across the endothelial surface layer that defines vascular integrity, so that the presence of this layer should be the basic requirement for a physiologic barrier function [32]. It was proposed that the endothelial glycocalyx acts as a primary molecular filter and generates the effective oncotic gradient within a very small space [38]. Transcapillary fluid exchange seems not to depend on the global difference between hydrostatic and oncotic pressure between blood and tissue. Rather, the hydrostatic and oncotic pressures between the blood and the small space directly underneath the endothelial glycocalyx, but still inside the anatomical lumen of the vessel, are decisive here [32, 40]. Taking the endothelial surface layer into consideration, the “classic” Starling equation [FIG. 19] was modified into the following:

Jᵥ=K_{f}([P_{c}−Pᵢ]−σ[πₑₛₗ−π_{b}])

where Jv—net filtration; Kf—filtration coefficient; Pc—capillary hydrostatic pressure; Pi—interstitial hydrostatic pressure; σ—refection coefficient for the plasma proteins; πₑₛₗ—oncotic pressure within the endothelial surface layer; and π_{b}—oncotic pressure beneath the endothelial surface layer.

All this indicates a dependency between an alteration of the endothelial surface layer and protein or colloid shifting toward the interstitial space. Destruction of the endothelial surface layer and, therefore, the vascular barrier, leads back to the conditions proposed by the classic Starling equation, entailing transcapillary fluid shifting to equalize hydrostatic and oncotic pressures between tissue and blood—a catastrophe, if the interstitial colloid osmotic pressure equals that of the plasma [40]. This implies that, the endothelial glycocalyx should be preserved to inhibit a pathologic fluid shift into the interstitium. Ischemia—reperfusion, proteases, tumor necrosis factor, oxidized low-density lipoprotein, and atrial natriuretic peptide have the power to degrade the endothelial glycocalyx [40]. While surgical stress itself is well known to cause release of several inflammatory mediators [41], atrial natriuretic peptide release is triggered by iatrogenic acute hypervolemia [42]. Obviously, despite not being easy to achieve, maintaining intravascular normovolemia could be the key to protect the endothelial glycocalyx beyond the hardly avoidable damage caused by inflammatory mediators due to trauma and surgery. This could minimize pathologic fluid and protein shifts toward the interstitium via preservation of the endothelial glycocalyx [40].

(e) Under the prior art, in addition to adjusting the fluid intake-elimination balance, the body strives to maintain the physiologic or homeostatic target volume of blood and fluid spaces by distributing fluid over central and peripheral functional fluid spaces. Transcapillary fluid filtration and absorbtion in microcirculation beds along with fluid and protein turnover in the lymphatic loop are very important for the above processes [20]. Although capillary is the principal site for the exchange of gases, water, nutrients and waste products serving the needs of the perfused site, the transcapillary fluid shifts may serve the needs of the circulation. In example, excessive plasma fluid may be deposited in the perfused tissues, or recruitment of fluid may be activated in the states of plasma dehydration or severe volume deficit (hypovolemia). The morphology and local regulatory mechanisms of the microcirculation are designed to meet these particular needs, but the structure and function of the microcirculation may be quite different from one tissue to another [20]. The microcirculation [FIG. 20] is defined as the blood vessels from the first-order arteriole to the first-order venulae. Although the details vary from organ to organ, the principal components of an idealized single arteriole and venulae are extended by the network of true capillaries [20]. Sometimes a metarteriolae—somewhat larger than the capillary—provides a shortcut through the network. The skin which is the largest organ in the body has an unusual feature—the numerous arteriovenous anastomoses in its apical regions. These metarteriolae are under neural control rather than the control of local metabolites. Maximal sympathetic activation can completely obliterate anastomotic vessels, therefore greatly reducing blood flow to the skin. Dermis is also the major site of high fluid compliance interstitium that can accumulate large amount of fluid. That is very important since derma under the skin of the nail [FIGS. 21-23] is conventionally used for observation of microcirculation. The thin nail plate is separated by a thinner nail matrix from a relatively thick layer called the subungual corium or the nail bed. The nail plate is an avascular structure. In the subungual corium there is an exceedingly dense plexus of blood-vessels [43]. They supply the overlying nail matrix and also the numerous arteriovenous anastomoses found in the nail bed. They are especially important for the thermoregulation. This vascular plexus arises from a large number of longitudinally running vessels which in turn arise from two or three vascular arcades that run transversely across the dorsal surface of the phalanx in the deeper layers of the nail bed. There are usually two or three subungual arcades [43]. Both arteriole and venulae have vascular smooth-muscle cells. Precapillary sphincters at the transition between a capillary and either an arteriole or a metarteriolae [FIGS. 20,23], control the access of blood to particular segments of the network. Sphincter opening or closure can create small local pressure differences that may even reverse the direction of blood flow in some segments. The capillary beds of most organs perform ultra filtration at the arteriolar end and reabsorption at the venular end [FIG. 24]. Capillary blood pressure falls from the arteriolar to the venular end. However, the midcapillary pressure is not the mean value, and it is not constant and uniform either [20]. Capillary pressure differs markedly among tissues. For example, the high pressure (−50 mmHg) in the glomerular capillaries is required for ultra filtration, meanwhile the pulmonary capillaries have unusually low pressure (−5-15 mmHg) minimizing ultra filtration that otherwise would lead to the accumulation of edema fluid in the alveolar space (pulmonary edema) [20]. The active contraction of vascular smooth muscle regulates precapillary resistance, which controls capillary blood flow. Modulating the contractility of vascular smooth muscle cells in precapillary vessels is the main mechanism for adjusting perfusion of particular tissue. In contrast to skeletal muscles, vascular smooth muscle cells receive multiple excitatory as well as inhibitory inputs. Moreover, these inputs come not only from chemical synapses (neural control), but also from circulating chemicals (humoral control) [20]. However, local control mechanisms can override neural or systemic humoral influences: tissue metabolites can regulate local blood flow in specific vascular beds, independently of the systemic regulation. Because blood flow itself can influence the local concentration of the metabolic intermediates, blood flow in capillary beds tends to oscillate spontaneously over time [FIG. 25] in a process known as vasomotion [20]. Thus, despite large changes in systemic arterial pressure the capillary beds maintain local flow within a narrow range because of autoregulation [20]. Meanwhile, fluctuations in metabolism contribute to the periodicity of vasomotion. Homeostasis acts similarly at the level of a single cell and systemic parameters that affect the whole body. Thus, accommodations of sympathetic stimulation in response to the changes in blood volume will similarly affect the systemic arterial tone [FIG. 26] and the tone of arteriolar sphincters in the capillary beds. Consequently, that will affect the midcapillary hydraulic pressure gradient and the related transcapillary fluid movement accordingly. In example, the arteriolar constriction or venular dilation reduces midcapillary transmural pressure gradient, while the arteriolar dilation or venular constriction increases it [20]. In the former case the hydraulic pressure dependent fluid filtration decreases, but it increases in the later setting.

(f) Under the prior art, lymphatics arise in the interstitium as small thin-wall channels that join together to form increasingly larger vessels [FIG. 27]. Lymphatics return the excessive interstitial fluid to the blood, thus completing local fluid balance [20]. Lymphatics are absent from some tissues, such as myocardium and brain, while they are most prevalent in the skin, respiratory and gastrointestinal tracts. The large lymphatics ultimately drain into the left and right subclavian veins. Filtration at the arteriolar end of capillaries exceeds reabsorption at the venular end by 2 to 4 liters per day, but fluid does not accumulate in the interstitium, because the excess of fluid and protein move into lymphatics [20]. Since it is dependent on interstitial pressure, the lymphatics normally returns the excessive interstitial fluid and protein to the circulation. Interstitium exhibits variable compliance, thus fluid added to the interstitium in its low-compliance range induces lymphatic efflux nicely matching excess capillary filtration [20]. If interstitium is already expanded, it is in high-compliance state [FIG. 28]. Then the lymphatic return does not compensate well for excess capillary filtration, so that interstitial fluid volume increases further. Lymphatic loop returns to the venous vascular system the 2-4 liters of fluid per day that represents the difference between transvascular filtration and reabsorption [FIG. 29]. Only a very small amount of filtered protein returns to the circulation by solvent drag. Nearly all of the filtered protein depends on the convective lymphatic loop for its ultimate recovery [20]. Normal control of interstitial fluid volume is obtained via changes in interstitial hydrostatic and colloid osmotic pressures, which will counteract changes in capillary fluid filtration and aim at restoring normal filtration, a phenomenon that may be termed ‘autoregulation’ of interstitial fluid volume. As for interstitial fluid pressure (P_{if}), the rise in pressure will be determined by the interstitial compliance (Cᵢ), defined as the ratio between the change in interstitial fluid volume (ΔVᵢ) and corresponding change in interstitial fluid pressure, i.e. Cᵢ=ΔVᵢ/AP_{if}. The normal relationship between volume and pressure is shown in FIG. 28. The volume-pressure relationship is linear (i.e. compliance is constant) in dehydration and in the initial stage of overhydration. When Vᵢ increases above control volume, the volume-pressure curve levels off implying that compliance increases. At over hydration above Vᵢ, compliance is virtually infinite because there is no increase in P_{if} as Vᵢ increases. At excessively high volumes compliance may again decrease because of restraints offered by fascias, capsules, etc. The final factor contributing to maintenance of interstitial fluid volume is lymph flow, which will increase and decrease with Pif and Vi. Filling of the initial lymphatics requires a pressure gradient, and although the mechanisms for formation of lymph are still not fully understood, P_{if} is one of the two pressures determining the pressure gradient from the interstitium to the initial lymphatic. It is considered a filling pressure for the initial lymphatics, and will thereby contribute to maintain a constant Vi. To summarize, the resulting changes in interstitial fluid hydrostatic and colloid osmotic pressure as well as lymph flow to changes in capillary filtration are such that alterations in interstitial fluid volume will be limited, a phenomenon that may be described as ‘autoregulation’ of Vi.

(g) Under the prior art, fluid distribution is evaluated by the methods based on mass balance which is an application of conservation of mass to the analysis of physical systems (measuring rate of uptake or elimination of a substance by an organ) [20]. By accounting for material entering and leaving a system, mass flows can be identified which might have been unknown, or difficult to measure without this technique. The general form quoted for a mass balance is as follows: the mass that enters a system must, by conservation of mass, either leave the system or accumulate within the system. Mathematically the mass balance for a system without a chemical reaction is as follows:

Input=Output+Accumulation

In the absence of a chemical reaction the amount of any chemical species flowing in and out will be the same. They use baseline measured or assume normal calculated blood volume for the evaluation of fluid infusion induced plasma dilution reflected by changes in blood hemoglobin concentration or other tracers. The dye-dilution method is a variation of the Fick procedure which is the simultaneous downstream measurement of the injected substance [20].

(h) Under the existing art, the very accurate method for dynamically investigating plasma volume as part of central expandable fluid space is based on volume kinetic analysis [44]. The method has been a subject of over 40 publications in journals of anesthesia and intensive care. The theory behind it explains the movement of fluid between functional body tissue compartments in relation to their expandability. It is a new innovative application of pharmacokinetic data analysis, earlier applied to drug disposition. The distribution of the fluid infused is modeled separately for each subject using a kinetic model based on the assumption that the volume of the fluid space expanded by the infused fluid strives to be maintained in a way similar to an elastic balloon [FIG. 30]. Serial changes in hemoglobin concentration serve as indicators of plasma dilution. After infusing the test fluids, the non-linear regression of fluid-induced changes in hemoglobin concentration is used to categorize mathematically the clearance curves as one, two or three volume of fluid space (1,2,3-VOFS) models. Mathematical models were built on that basis to represent the changes in volume of the body fluid spaces associated with intravenous administration of different solutions. Input data for mathematical parameter estimations were dilution of blood, measured as reduction of blood hemoglobin concentration [44]. In contrast to mass balance, baseline plasma volume measurement is not necessary for volume kinetic analysis. Both the effect of fluid bolus on plasma dilution and rate of infusion needed to maintain the given level of dilution are predicted by kinetic modeling. The estimated value of baseline blood volume is used for corrections of dilution due to blood sampling.

(i) Under the existing art, the activated plasma fluid equilibration in the well perfused capillary beds is complete in about 20 minutes after the end of infusion. It was demonstrated numerous times in volunteers [44-47] and in patients [48]. That timecourse was later supported by measurements of arterio-venous difference in plasma dilution in volunteers who received 15 mL/kg of lactated Ringer's solution over 10 minutes [49]. More specifically, the arterio-venous dilution difference (avDD) was used to define the direction and the time-course of the fluid flux between plasma and the interstitial fluid space in the vicinity of the venous sampling site, whereas volume kinetics was used to indicate the direction of the fluid flux for the whole body. The arterio-venous plasmadilution difference in the forearm became negative in 2.5 min after the infusion ended. Meanwhile, for the whole body, the kinetic calculations showed that the mass flow of fluid does not change direction from tissue to plasma until about 20 min later [49].

(j) Under the prior art, the US patent pending HBS Method and the related homeostatic blood states theory [27-28] are used for the evaluation of preexisting fluid status and its optimization. The theory and the HBS method consist of five discovered concepts: 1) The homeostasis of the human body strives to maintain the red cell mass specific homeostatic target blood volume. It is equal to ideal (normal) blood volume and maintains ideal plasma volume only once per physiologic hematocrit scale—at hematocrit of Ideal Total Match. 2) At physiologically critical—lowest and highest—hematocrit limits target states maintain the Maximal Target Deviation, which is equal to Constant k. It is the maximal homeostatically acceptable sum of target state specific absolute volume deviations in respect to ideal blood and plasma volumes. 3) Plasma hydration limits in respect to target states are reached, when either blood or plasma volume overcome ideal values by the Maximal Safe Deviation, which is half the value of Constant k. 4) Compensatory osmotic accommodations are homeostatically induced to oppose the advanced deterioration of plasma hydration that overrides the MSD limits or target states maintain similar blood and plasma volume deviation patterns in respect to ideal blood and plasma volume. 5) Target tissue perfusion focused vasomotor tone maintains target tissue perfusion despite different patterns of target blood volume. Vasomotor tone under the control of intact sympathetic stimulation and homeostatic guidance is considered as normal or target tissue perfusion focused by the new theory. It is considered ideal only when ideal blood volume is present, which is inherent to target states at Ideal Total Match hematocrit, but may be reached in a variety of deviations from target states at other Hct values.

(k) Under the existing art, based on the theoretical framework of the US patent pending HBS Method and the related homeostatic blood states theory [27-28], an optimized plasma hydration state determined by VLT is consistent with the upper limit of the normal or physiologic range of plasma hydration fluctuations. In that state, plasma would presumably hold the maximal load of ‘free water’ in the sense of its bioavailability for tissues in the processes of hydration. It is assumed to be a colloid-unbound fluid. The first study aiming to validate the VLT method was made on volunteers, who had preexisting fluid deficit [50]. As expected according to the VLT method dehydrated volunteers presented with a more pronounced hemodilution measured after the 30 min steady state following crystalloid loading than those with preexisting normal fluid status. Such a shift to a new baseline could not be explained by conventional volume kinetics, because the later assumes that the physiologic target volume of the fluid spaces is the baseline status before the fluid load, so the body strives to return to that volume soonest [44-49].

(l) Under the existing art, e.g. U.S. 2007/0178167 A1, the HBS Method and the related homeostatic blood states theory proposed an indirect evaluation of changes in plasma osmolality from the dynamics of mean cell hemoglobin concentration (MCHC) [27-28]. The latter is associated with the dynamics of mean cell volume (MCV) and mean cell hemoglobin content (MCH). The MCV increase is assumed to be a reflection of the corresponding decrease in plasma osmolality (osm), and vice versa [51-52]. In the state of unchanging erythropoietic blood content, the MCH remains constant. Thus, MCHC changes are induced solely by osmotically induced changes in MCV. Consequently, the dynamics of plasma osmolality can be traced by changes in MCHC per se. The MCHC value is derived from ratio hemoglobin to hematocrit. Thus, changes in plasma osmolality are traceable from the dynamics of hemoglobin and hematocrit [51-52]. That is important for fluid management since changes in plasma osmolality can be induced even by infusing such “physiologic” fluids as Ringer's solution which is isotonic in vitro.

### Below Follow the Deficiencies Related to Existing Art 3[2.1.5]

2.1.6. However, the blood volume and its dilution/hydration, also the state of interstitial fluid content and expansion remain unclear in most clinical settings.

That is because:

(a) Direct measurements of blood volume are not applicable in the dynamically changing plasmadilution.

(b) Although a range of mathematical methods are used to calculate the normal blood volume, they fail to provide clinical benefit or even are misleading during the dynamically changing content of red cells and fluid.

(c) The red cell volume dependent estimates of blood volume based on the US patent pending Homeostatic Blood States method or HBS Method need validation.

(d) The classical Starling's equation for the transcapillary fluid exchange was used for decades, but recently it was revisited. That revision of the formula was required, because the previously unknown role of endothelial glycocalyx for the transcapillary movement of fluid has been recognized. It means that understanding of physiology of fluid distribution is still not complete.

(e) Although accommodations of sympathetic stimulation in response to the changes in blood volume are known to be similarly affecting the systemic arterial tone and the tone of arteriolar sphincters in the capillary beds, monitoring of midcapillary hydraulic pressure gradient and the related transcapillary fluid movement is not applied in clinical practice.

(f) The mass balance and Fick principle also the dye dilution methods are also invasive, cumbersome and require expensive, bulky monitors, and there may be a degree of variability in the measurements of cardiac output and fluid distribution.

(g) Methods based on volume kinetics have limited clinical applicability, mainly due to steady state requirements (no intravenous infusion or any other origin plasma dilution present) during the post infusion period when serial blood samples are taken. Volume kinetic parameters are vulnerable to loss of hemoglobin and post bleeding capillary refill origin plasma dilution. The predictions of fluid disposition with further infusions can be deteriorated by the naturally changing fluid balance and transcapillary fluid shifts. Although volume kinetic models do not require baseline blood volume for major estimates, they apply assumptions of normal calculated blood volume for corrections of dilution due to blood sampling.

(h) Arterio-venous dilution difference is subject to changes induced by both the fluid distribution and redistribution. However, continuous monitoring of arterio-venous dilution difference is clinically not applicable, because currently it can be done only invasively, and that would require dozens of blood samples.

(i) All five major concepts proposed by the US patent pending HBS Method and the related homeostatic blood states theory still need validation.

(j) The normal or physiologic range of plasma hydration fluctuations proposed by the U.S. 2007/0178167 A1 HBS Method and the related homeostatic blood states theory need validation.

(k) Indirect evaluation of changes in plasma osmolality from the dynamics of MCHC proposed by the US patent pending HBS Method and the related homeostatic blood states theory needs validation. Also, it did not take into account the possible redistribution of the average MCV because of different osmotic resistance of red cells in circulation. It may be the reason why MCV, which is an average red cell volume, may hypoosmotically decrease instead of expected increase. The MCHC will change accordingly.

### Existing Art 4

### Plasma dilution measurements

2.1.7. Under the existing processes, the blood hemoglobin concentration is probably the most frequently obtained blood test in both outpatients and inpatients. It serves as plasma dilution marker for the assessment of plasma retention of the infused fluid.

(a) Under the prior art, blood hemoglobin serves as endogenous tracer of plasma dilution. Hemoglobin dilution-time curves are used for the dynamical investigation of plasma dilution in human and animal studies [44-49]. Sometimes blood hematocrit is also used for the same purposes. Venous blood samples are usually deployed for analysis. Arterial hemoglobin obtained from an artery of the limb is assumed as being a reflection of the fluid status of the whole body [49]. Meanwhile, venous Hb obtained from the peripheral vein is assumed is affected by the intercompartment fluid balance of the limb, where the blood sample was taken [49].

(b) Under the prior art, the blood hemoglobin concentration is evaluated in arterial, venous and microvascular blood, invasively or non-invasively. In clinical practice, arterial and venous Hb is obtained only invasively, while microvascular—only noninvasively. A range of non-invasive Hb measurements has been put into test over the last decade [53]. In 2005, Masimo introduced Masimo Rainbow SET® Pulse CO-Oximetry™, a breakthrough noninvasive blood constituent monitoring platform that can measure many blood constituents that previously required invasive procedures. Masimo Rainbow SET continuously and noninvasively measures total hemoglobin (SpHb™), oxygen content (SpOC™), carboxyhemoglobin (SpCO®), methemoglobin (SpMet®), PVI®, and acoustic respiration rate (RRa™), in addition to oxyhemoglobin (SpO2), pulse rate (PR), and perfusion index (PI), allowing early detection and treatment of potentially life-threatening conditions [54].

(c) Under the prior art, total hemoglobin (SpHb™) is measured [FIG. 31] under the nail of the upper limb [54]. According to the anatomy and physiology of that site, the hemoglobin is actually being measured in the capillaries of the derma under the nail, on which the probe is being placed [43]. The apical regions of the derma have an unusual feature—the numerous arteriovenous anastomoses, which are under neural control rather than the control of local metabolites [20]. Maximal sympathetic activation can completely obliterate anastomotic vessels, therefore greatly reducing blood flow to the skin. Derma is a major site of high fluid compliance interstitium that can accumulate large amount of fluid [20].

(d) Under the prior art, the total hemoglobin (SpHb™) is assumed as being equal to arterial Hb [54]. The venous values are automatically calculated by a device relying on the findings from research reporting that arterial Hb measurements can be expected to be, on average, 0.7-1.0 g/dL less than the Hb measurements derived from venous blood [55-56]. The operation of the Masimo Rainbow SET® Pulse CO-Oximetry™ device [FIG. 32] that provides total hemoglobin (SpHb™) is based on Masimo SET Pulse Oximetry Technology with added Rainbow Technology Algorithms [54].

(e) Under the existing art, in research, the plasma dilution trends are statistically analyzed after pooling of data obtained from different subjects [44-49]. Based on that approach, the arteriovenous difference in plasma dilution during and after the crystalloid volume loading was analyzed in 9 healthy volunteers [49]. The conclusion was that arteriovenous dilution difference increases significantly and reaches maximum by the end of infusion, then it decrease significantly below baseline during the 20 min steady state. Moreover, the venous dilution significantly exceeding arterial and baseline remains similar even after another hour long steady state.

### Below Follow the Deficiencies Related to Existing Art 4[2.1.7]

2.1.8. However, under the prior art, markers of arterial and venous plasma dilution such as hemoglobin concentration (Hb) are obtained invasively, are not continuous and real-time. Acknowledging the extremely high rate of fluid exchange in capillaries the measurements of microvascular (capillary) hemoglobin concentration, e.g. total hemoglobin (SpHb™), needs as short as possible averaging time, also the better clinical interpretation of the readings, and better established dynamic correlation with arterial and venous hemoglobin concentration, and especially during fluid challenges and/or bleeding.

That is because:

(a) The clinical measurement of Hb has inherent variability [57-58]. Physiologic factors such as the blood source (venous or arterial), site and time of blood draws, and patient body position are recognized to add variability to hemoglobin levels. Blood draw techniques such as “pushing out” blood during a fingertip capillary draw and blood-mixing errors can have an additional variability impact on Hb measurement.

(b) Arterial and venous Hb can be obtained with most accuracy only invasively. Continuous monitoring of arterial and venous Hb is not available so far. Continuous noninvasive measurements of total hemoglobin (SpHb™) cannot be used as an alternative even because of vulnerability to the shifts of transcapillary fluid exchange since SpHb™ is obtained in capillaries. To the best knowledge off the inventor, the devices that can be used for measuring of capillary Hb such as Masimo Rainbow SET® Pulse CO-Oximetry™ make the mathematical conversion from assumed arterial to venous Hb values relying on the early investigations of arteriovenous differences in plasma dilution. More specifically, in the manufacturer's brochures, the findings of Mokken et. al. [55] and Yang et. al. [56] are cited. They reported that arterial Hb measurements can be expected to be, on average, 0.7-1.0 g/dL less than the Hb measurements derived from venous blood. However, most recent observations reported that arteriovenous plasma dilution difference can be shifting from positive to negative and vice versa during the intercompartment fluid equilibration. More specifically, arteriovenous dilution difference was found to decrease significantly after the intravenous fluid loading, and venous dilution may significantly exceed arterial even after an hour long steady state following infusion [49].

(c) Arterio-venous dilution difference is subject to changes induced by both the fluid distribution and redistribution. The major intercompartment fluid equilibration takes place in microvessels. These processes are very sensitive and may be extremely intense since 90% of the body fluid exchange involves only 10% of whole body blood which is found in systemic capillaries. However, to the best knowledge of the inventor, there is no method that would provide the clinical interpretation of microvascular Hb changes in that context. Although SpHb™ is obtained continuously in real time, there is no method for the SpHb™ contour analysis similar to that of the pulse contour analysis during fluid challenges.

(d) The evaluation of plasma dilution by means of pooled plasma dilution trends from different individuals may be misleading because every subject may have responded in the completely different context-sensitive way. However, the pooled data would show the sum effect that is far from any of these responses. For example, the statistically analysis after pooling the data obtained from 9 different subjects showed that arteriovenous dilution difference increased significantly and reached maximum by the end of infusion [49]. However, in contrast, half of the subjects demonstrated that arteriovenous dilution difference decreased and reached minimum by the end of infusion.

In conclusion, it is generally acknowledged that in everyday clinical practice we still cannot accurately identify and modify the individual hydration and volume statuses. Moreover, there is no scale applicable to the range of whole body hydration states. Relying on indirect signs of body hydration, tissue oxygenation and adequacy of effective circulating volume is a common clinical practice. Thus, defining “ideal” fluid replacement strategy remains a critical problem and ‘gold standard’ is still missing. As a consequence, current infusion therapy is challenged by inadequate body hydration, plasma over dilution and/or over hydration followed by coagulation disorders and circulatory overload that are associated with significant increase in morbidity and mortality. Thus, there is a need in the art for conventionally available, inexpensive and minimally invasive methods that would provide guidance for the fluid therapy preferably by using hemoglobin concentration since it is inexpensive and conventionally available. Techniques for obtaining arterial, venous and capillary Hb in a continuous real-time and noninvasive manner would be of great value for the evaluation of plasma dilution responsiveness simultaneously at different sites. Evaluation and clinical interpretation of the obtained trends could lead to the better diagnosis of fluid status and guidance of fluid management.

### BRIEF SUMMARY OF THE INVENTION

The present invention provides solutions to those deficiencies identified under the “Prior Art” in the BACKGROUND section of this application.

In particular, conventional perioperative derangements in fluid balance are common. Current goal directed fluid therapy aims for optimization of circulation via maximization of flow-related parameters by administering series of intravenous fluid loads—mainly colloid solutions—separated by 5 min periods without any fluid administration or intake. However, this method aims for maximization of cardiac output and related improvement of circulation without evaluation and modification of the body hydration. That sets a case for the possibility of circulatory optimization or, even more likely, the maximization in expense of deterioration of the whole body hydration status. The related fluid overload of interstitial tissues is concern even when colloid solutions are infused since, despite the fact that they are pure blood volume expanders, the extravasation of free water takes place when the blood volume starts exceeding the physiologic target volume. Meanwhile, evaluation of the degree of interstitial hydration referred to as interstitial hydration status (hydration later in the text) remains challenging, and deteriorations usually remain undetected until they become severely advanced. In addressing these deficiencies within existing state of art, the volume loading test (VLT) for evaluation and modification of the whole-body hydration was proposed in 2006. This method was applied to our earlier research data, and support for its validity was reported. Nevertheless, it remains cumbersome, also lacks sensitivity, specificity and appropriate clinical applicability and interpretation. Thus, it is still impossible in everyday clinical practice to accurately identify and individually modify the hydration.

Addressing deficiencies within existing methods, the current invention proposed the minimal Volume Loading Test (mVLT) method. The mVLT method implies but is not limited to individual evaluation of plasma dilution response to intravenous administration of consecutive relatively small test volume loads of isoosmotic isooncotic crystalloid solutions (test bolus later in the text) followed by 5 minute periods without fluid administration and intake (5′StS or 5 min steady state later in the text). The current invention proposed the mathematical model of bolus induced response of deviations (BIRD-math) for the evaluation of response to the fluid challenges (fluid responsiveness) during mVLT. Also, the physiologic model of bolus induced response of deviations (BIRD-phys) was proposed for the explanation of physiologic processes behind the diagnostic criteria applied for the clinical evaluation of the mVLT results. In contrast to the existing state-of-the-art within goal directed fluid therapy, arterial and capillary hemoglobin concentration (Hb) is used as main target parameter for the guidance of intravenous fluid or diuretic administration. The venous plasma dilution is highly dependent on the ratio of capillary flow to blood flow via arterio-venous anastomoses which is changing dynamically as affected my multiple factors. Thus, evaluation of venous plasmadilution trends has limited reliability for the evaluation of the mVLT results. Meanwhile, trends of capillary plasmadilution in a single capillary bed in derma can be used instead of arterial trends because mVLT method evaluates the deviation of variables from the dynamically changing baseline, and thus the inherent difference between the arterial and capillary Hb has insignificant impact on mVLT result evaluation. Moreover, the transcapillary filtration-absorption ratio related shift in capillary plasmadilution of a single dermal capillary bed is a close reflection of the simultaneous shifts of other similarly perfused capillary beds in derma and skeletal muscles. Thus, arterial plasmadilution trend which is a reflection of the whole-body transcapillary filtration-absorption ratio related shift in plasmadilution is very close to capillary plasmadilution trend of a single dermal capillary bed. When, as a result of increasing degree of the whole body hydration, the arterial plasma dilution trends become significantly affected by the dynamics of the transcapillary filtration-absorption ratio in the newly opening capillary beds such as splanchnic, the fluctuations of filtration-absorption ratio of a single dermal capillary bed are already negligible since well-hydrated adjacent interstitium has reached the state of low fluid compliance. Thus, arterial plasmadilution fluctuations are further well reflected by plasmadilution fluctuation in a single capillary bed since influence of local changes in filtration-absorption ratio is insignificant. Moreover, fluctuations of capillary plasmadilution can serve for the indirect monitoring of simultaneous changes in cardiac stroke volume, since the latter is affected by both—the plasma dilution induced increase of venous return (pre-load) and the arteriolar tone (after-load), which is an important determinant of the midcapillary filtration-absorption ratio that affects capillary plasmadilution.

The diagnostic mVLT algorithm (DmVLT) guides the sequence of processes aiming to determine the preexisting hydration status. The optimizing mVLT algorithm (OmVLT) guides the sequence of processes for the modification of hydration, volume status and oxygen transport. The current invention also employs an indirect assessment of cardiac stroke volume response to the fluid challenges during mVLT from the dynamics of plasma dilution. Context-sensitive optimization of plasma dilution is associated with the corresponding optimization of cardiac stroke volume according to the mVLT method. At that point, administration of sympatomimetic drugs and, in case of hypovolemia, the colloid infusion is recommended aiming for the maximization of stroke volume. Then red cell transfusion is recommended if there are persistent signs of inadequate oxygen delivery to tissues, i.e. increased concentration of arterial lactates. Switching from fluid loading to transfusion is part of the OmVLT algorithm.

In contrast to the existing state-of-the-art within goal directed fluid therapy, the Hb is used as main target parameter for the guidance of intravenous fluid or diuretic administration. Nevertheless, the Multimodal Feedback Loop (MFL) method deploys simultaneous analysis of different target parameters' response to fluid challenges during mVLT by analyzing their deviations by processing the measured values with equations of the BIRD-math. That allows simultaneous evaluation and modification of cardiovascular performance and body hydration.

The MFL method can be used in the software of a proposed Multimodal Feedback Loop device (MFL device). The device provides processing of manual or automated input data which includes but is not limited to flow and plasma dilution related target parameters. Output data includes but is not limited to the conclusions concerning the preexisting and current body hydration, also the most likely pattern of cardiac stroke volume response to the fluid challenges. Interface with an automated continuous real time invasive and/or noninvasive capillary Hb monitoring devices, fluid infusion/transfusion pumps and total intravenous anesthesia (TIVA) or target controlled infusion (TCI) devices forms an autonomic self-regulating system operating on the feedback loop principle. This allows more efficient, cost effective and safe management of fluid and transfusion therapy.

A mVLT method is provided for determining and optimizing the state of hydration of interstitial tissues of the human body comprising:
a) quantifying the subject's initial baseline generic target parameter(s) that include but are not limited to arterial, venous and capillary Hb, but the minimum set of target parameters for the monitoring of hydration is capillary Hb in a single or multiple measuring sites;
b) intravenously administering an intravenous relatively small test volume load—from 1.5 to 2.5 ml per kg of subject's lean body mass—of fluid to the subject—an isooncotic, isoosmotic non-cellular liquid is infused at the highest available and clinically appropriate rate with the target of infusing it over a period of 2-5 minutes (test bolus);
c) quantifying the subject's generic target parameter(s) after a period of 5 minutes from the end of step b), but before 6 minutes from the end of step b) without further intravenous administration of a liquid to the subject;
d) referring to the processes in steps a), b) and c) as the 1^{st} minimal volume loading step or just 1^{st} step;
e) determining by formulae of the BIRD-math the derivative target parameter(s) for the respective generic target parameter(s) obtained in steps a) and c) (TAB.3);
f) repeat the processes described in steps b) and c), and refer to that as 2^{nd} minimal volume loading step or just 2^{nd} step;
g) determining by formulae of the BIRD-math the derivative target parameter(s) of the respective generic target parameter(s) obtained in step f) (TAB.3);
h) iteratively repeating steps b) and c) until 4 steps are complete or until the diagnosis of hydration is derived (TAB.3) in compliance with the Diagnostic mVLT algorithm
(DmVLT) as described in FIG. 45, and/or until optimization of hydration is reached according to the Optimizing mVLT algorithm (OmVLT) as described in FIG. 46;
i) Progressively increasing iv test bolus when the volume every next test bolus exceeds the previous load by about 25%, but the maximal recommended load is 5.0 ml kg⁻¹, which can be applied in the states of massive hemorrhage or shock.
j) Diuretics can be injected intravenously instead of test fluid bolus if the release of the edema is required; the specific deviations of plasma dilution will be seen if evaluated by the mVLT and the BIRD-math; initially, the plasma dilution will be induced by the diuretic induced flux of fluid from interstitium into blood vessels; then the plasma concentration will be seen as fluid is being excreted by kidneys; diuretic injections should be continued until the diuretic-induced plasmadilution becomes negligible.

A multimodal feedback loop device (computing apparatus) is provided comprising:
a) An electronic input-link with the device that can provide the capillary blood hemoglobin concentration input from a single or multiple measuring sites to the computing apparatus;
b) an electronic input-link with the device that can provide the arterial blood hemoglobin concentration input to the computing apparatus;
c) an electronic input-link with the device that can provide the venous blood hemoglobin concentration input to the computing apparatus;
d) an electronic input-link with the device(s) that can provide input of target parameters other than hemoglobin concentration, e.g. stroke volume, to the computing apparatus;
e) the computing apparatus that has manual input of parameters' values available from the other devices or laboratory analysis reports;
f) the computing apparatus that processes the input variables by equations of the BIRD-math, then analyses the derived variables (TAB.3) and their dynamics according to the diagnostic criteria of the mVLT method set forth in the specification and figures, provides the diagnostic information on the display to the operator, suggests further actions and awaits for the confirmation from the operator, sends commands to the infusion pump accordingly, e.g. to administer another test bolus or to switch to maintenance fluid administration;
g) the computing apparatus comprising the memory, which memory is communicatively coupled to one or more processors, the memory comprising at least one sequence of instructions which when executed by the processor causes the processor to perform the determination steps and comparison steps of the methods described herein above so as to provide an output to an intravenous fluid pump controller;
h) as an option, the intravenous fluid pump controller which is attached to, and controlled by output from, the computing apparatus.

A non-transitory computer-readable storage medium with an executable program stored thereon, wherein the program instructs a microprocessor to perform the steps a) through i) of claim the first or second method recited above. In an embodiment the steps of intravenous administration are performed by sending a signal to a device so as to affect intravenous delivery of the liquid by the device to the subject.

The dynamics of various parameters can be evaluated by processing the corresponding data based on the equations of the BIRD-math. Examples of such parameters include flow-related parameters such as perfusion index, pleth variability index (PVI), tissue hemoglobin content, cardiac stroke volume, global end diastolic volume index, end diastolic filling ratio, and others.

### Diagnostic criteria in the mVLT method

Based on the above, the diagnostic criteria for the monitoring of changes in the degree of hydration are proposed by the inventor:
1. Increasing positive values of a single capillary-bed derived response variation (VPR) and supported by increasing responsiveness (ABS-SRV) are markers of the on-going transitory optimization of interstitial hydration in that single capillary-bed-dependent interstitial tissues and the whole body.
2. Slowly decreasing positive values of a single capillary-bed derived response variation (VPR) and supported by decreasing responsiveness (ABS-SRV) are markers of the trend towards transitory maximisation of interstitial hydration in the single capillary bed-dependent interstitial tissues but maximisation may not be reached in the interstitium of the whole body (since capillary-bed-dependent interstitium reaches the maximal interstitial fluid compliance earlier that interstitium of the whole body; thus, maximisation of the VPR value in arterial trends may not be complete at that point).
3. Steeply decreasing positive values of a single capillary-bed-derived response variation (VPR) and supported by decreasing responsiveness (ABS-SRV) is a marker of the shift towards transitory maximization of interstitial hydration in the whole body.
4. Aiming to transform the transitory optimised state of hydration into relatively permanent, there are two options: (a) after reaching the criteria of transitory maximization, the maintenance infusion of crystalloids is set up to maintain the recorded at that point capillary Hb value, or (b) the mVLT steps should be continued until the minimization of the decreasing positive VPR supported by the decreasing responsiveness (ABS-SRV), and then make a 20 pause in fluid administration, trace the changes in capillary Hb and, when it returns to the value previously recorded in the step consistent with criteria of transitory maximization, initiate the maintenance infusion of crystalloids which is set up to maintain the recorded at that point capillary Hb value, or (c) the maintenance fluid infusion has to be tailored to maintain the plasma dilution reached at a specific mVLT step.
5. Baseline state's hydration status is determined when the first criteria of the transitory hydration state are met: (a) the increasing positive VPR and related diagnosis of transitory rehydration going on determines the diagnosis of baseline dehydration; (b) the decreasing positive VPR and related diagnosis of transitory overhydration going on determines the diagnosis of baseline normohydration; (c) the minimized positive VPR and related diagnosis of severe transitory overhydration going on determines the diagnosis of baseline moderate to severe overhydration.
6. Two consecutive negative VPR defines the hydration status within the hydration plateau, and normally will be followed by positive VPR, but the persistently negative VPR is associated with severe blood loss (persistent extravasation by entering the lymphatic loop for the endogenous recovery of plasma volume via fymph influx) or transcapillary leak, e.g. sepsis. When hemorrhage related demands for interstitial fluid are satisfied or septic extravasation of fluid resides, the VPR becomes positive.
7. Indirect evaluation of cardiac stroke volume's responsiveness: (a) increase in stroke volume is associated with arteriolar tone decrease and/or venular tone increase, and thus it will be seen as positive capillary VPR value; (b) decrease in stroke volume will be associated with arteriolar tone increase and/or venular tone decrease, and thus it will be seen as negative capillary VPR value.
8. Supportive interpretation of perfusion index (PI) and pleth variability index (PVI): (a) the PI increase associated with positive VPR means that capillary flow increases, arteriolar tone increases or constant, venular tone decreases or constant, stroke volume decreases or constant, arterio-venous shunting ratio-constant, transcapillary filtration-absorption ratio decreases, and finally—the PVI is high and/or increasing; (b) the PI decrease associated with positive VPR means that capillary flow increases, arteriolar tone increases or constant, venular tone decreases or constant, stroke volume decreases or constant, arterio-venous shunting ratio decreases, transcapillary filtration-absorption ratio decreases, and finally—the PVI is constant (low or high depending on the amplitude of fluctuations in PI and capillary flow); and (c) the PI decrease associated with negative VPR means that capillary flow decreases, arteriolar tone decreases or constant, venular tone increases or constant, stroke volume increases or constant, arterio-venous shunting ratio—constant, transcapillary filtration-absorption ratio increases, and finally—the PVI is decreasing. Other combinations apply according to the same principles that are based on the assumption that increasing capillary flow via decreased arteriolar tone and/or increased venular tone leads to significant increase of PI and decrease of capillary Hb marked by positive VPR.
9. The severe to moderate dehydration specific patterns are also the markers of hemorrhage, especially if preceded by the optimizing mVLT.
10. Increasing arterio-capillary dilution difference is the sign of increasing arterio-venous shunting.

It should be emphasized that the major diagnostic criteria described above (e.g. described within the “Diagnostic criteria in the mVLT method” section) are applicable to all of arterial, capillary and venous trends.

### BRIEF DESCRIPTION OF FIGURES AND TABLES IN THE APPENDIX

FIG. 1: Major strategies of fluid management (5).

FIG. 2: Conventional algorithm for the goal directed fluid administration aiming for maximization of flow related target parameters. The high rate (infused over 2 min) small volume (usually 200 ml) intravenous fluid load (usually colloid) is commonly deployed.

Target parameter's response is evaluated after 5 min following the fluid challenge. Its increase for more than 10% justifies another fluid load. Optimization of volume status and maximization of target parameter is assumed when target parameter's increase is <10% (5).

FIG. 3: Static flow-related parameters of cardiac preload. Global end-diastolic volume index (GEDVI) represents the other volumetric measures such as right ventricular end-diastolic volume (RVEDV), left ventricular end-diastolic area (LVEDA) and intrathoracic blood volume (ITBV) (5).

FIG. 4: The GDM algorithm part A. Baseline evaluation of fluid status. GDM-goal directed management in fluid administration, SV—cardiac stroke volume; SVV—stroke volume variation; PPV—pulse pressure variation (during the cycle of mechanical ventilation) SV—stroke volume; SVV, PPV—stroke volume and pulse pressure variation; EVLWI—extravascular lung water index (5).

FIG. 5: The Frank-Starling curve of the heart. In GDM strategy, the fluid load induced maximization of stroke volume is associated with the highest point on the Frank-Starling curve. The dynamic flow related parameters (SVV and PPV) exceeding 10% are predictors of significant SV increase in response to fluid loading since they indicate the position on the rising slope of the Frank-Starling curve (5).

FIG. 6: Strength of evidence in the support of the conclusions derived from baseline evaluation of fluid status. The stronger the sum evidence, the heavier the line connecting associated sources of evidence. The strongest evidence is provided by the combination of consistent findings derived from basic clinical evaluation, volumetric measures of preload and dynamic flow related parameters, especially those defining stroke volume responsiveness (SVV) (5).

FIG. 7: The GDM algorithm part B. Optimization of fluid state by evaluating the stroke volume response and, optionally, preload response to intravenous colloid fluid challenge (5).

FIG. 8: The GDM algorithm part C. Maintenance of optimized fluid status and optimization of oxygen transport (5).

FIG. 9: The Frank-Starling's curve of the heart. The fluid load induced maximization of stroke volume associated with the highest point on the Frank-Starling's curve may be shifted by inotrops or transfusion: the location on the peak of the curve (A1) may be shifted to the rising part (A2) with a potential to reach a higher peak (A3) of the curve (5).

FIG. 10: The circulatory beds of the human body [19].

FIG. 11: Venous blood compartment: the stressed and unstressed volumes (tub analogy) [21]. Water in a tub represents total blood volume. A hole in the wall of the tub between the surface of the water and the bottom of the tub divides total volume into stressed (Vs) and unstressed (Vu) volumes, above and below the hole, respectively. The water leaves the tub through the hole at a certain rate that depends on the diameter of the hole (which would reflect venous resistance [VenR]), and on the height of the water above the hole, representing Vs; the larger the Vs, the higher the flow through the hole. The water between the hole and the bottom of the tub does not affect the flow of water through the hole; this is the Vu, a sequestered volume that does not directly participate in the rate of water flow (venous return). With the same amount of water in the tub (total blood volume in the venous system), the relation between Vs and Vu can be changed by moving the hole up or down. Moving the hole down represents venoconstriction and increases Vs (and venous return). The distal end of the tube, attached to the hole in the tub wall, represents central venous pressure (CVP): the higher the distal end, the higher the CVP and the lower the pressure gradient for venous return, and vice versa. The inflow tap represents the arterial flow. The hydraulic disconnect between the tap and the tub represents functional disconnection between the two (arterial flow and the venous system) due to high arterial resistance.

FIG. 12: Integrated response to hemorrhage.

FIG. 13: Two-compartment model [21]. Inside: The two circuits represent two compartments; the solid lines represent arterial and noncompliant venous compartments (the main, basic circuit). Dashed lines represent arterial and compliant (splanchnic) venous compartments. The compartment with compliant veins is outside of the main circuit. Therefore, changes in arterial or venous resistance in compliant compartment do not directly affect arterial or venous resistance in the main circuit with noncompliant veins. Thickness of the lines reflects the amount of flow within the vessels under normal conditions. The size of the junctions between arteries and veins reflects the blood volumes contained in the two circuits. Outside: Effects of change in arterial resistance feeding compliant (splanchnic) and noncompliant (muscle) veins. SplArtR and NonSplArtR represent arterial resistance in arteries feeding compliant and noncompliant veins, respectively. F, P, and V represent flow, pressure, and volume within the venous vasculature, respectively. AP—arterial pressure; MCFP—mean circulatory filling pressure; VR—venous return. Change in resistance in arteries feeding splanchnic and nonsplanchnic vasculature leads to changes in venous return in opposite directions through changes in flows, pressures, and intravenous volumes (see text for explanation).

FIG. 14: Interaction among cardiovascular subsystems and non-circulatory systems [20]. GI-gastrointestinal tract.

FIG. 15: The branching of the capillaries [20].

FIG. 16: Transcapillary fluid exchange. Interaction of multiple factors in the processes of transcapillary fluid exchange [20].

FIG. 17: The vascular barrier and the transcapillary fluid exchange according to classic Starling's equation. A. The classic description of vascular barrier in the capillaries according to Starling. An inward-directed colloid-osmotic (oncotic) pressure gradient is opposed to an outward-directed hydrostatic pressure of fluid and colloids. The arrows symbolize the small net fluid filtration assumed according to this model. The extremely simplified illustration does not consider the postulated small net fluid reabsorption on the venular site suggested by this model, due to an assumed decrease in the hydrostatic and an assumed increase in the oncotic pressure gradient. The Starling equation is mentioned in the main text. ΠI—oncotic pressure in the interstitial space; ΠV—oncotic pressure in the vascular lumen; EC—endothelial cell; PV—hydrostatic pressure in the vascular lumen; PI—hydrostatic pressure in the interstitial space. B. The simplified description of vascular barrier's function according to Starling's equation. [32,33]

FIG. 18: Endothelial surface layer: the distribution space for the endothelial glycocalyx bound proteins. Schematic drawing of the distribution space for (A) artificial colloids (e.g., hydroxyethyl starch), and (B) natural plasma proteins (e.g., albumin) at the vessel wall within the first minutes after injection. [33]

FIG. 19. The “classic” and the “revised” Starling's principle. A comparison.

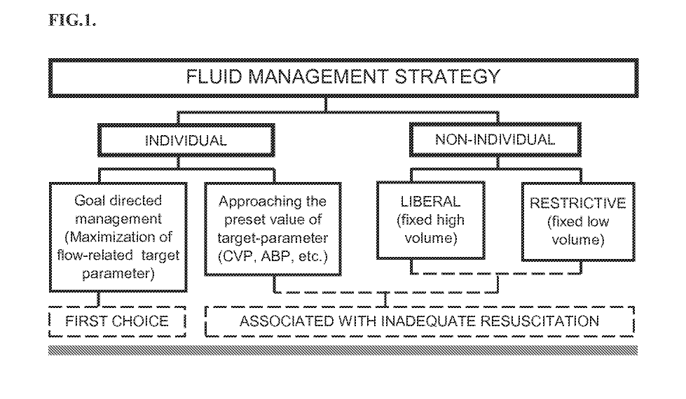

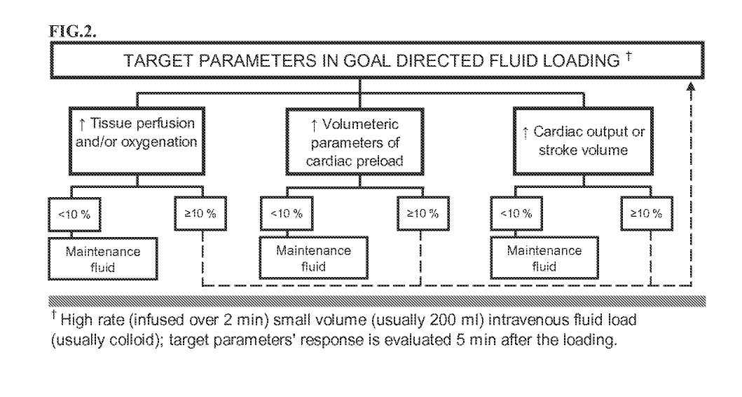

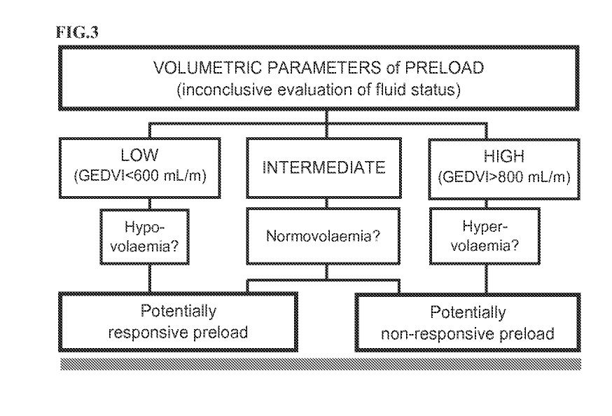

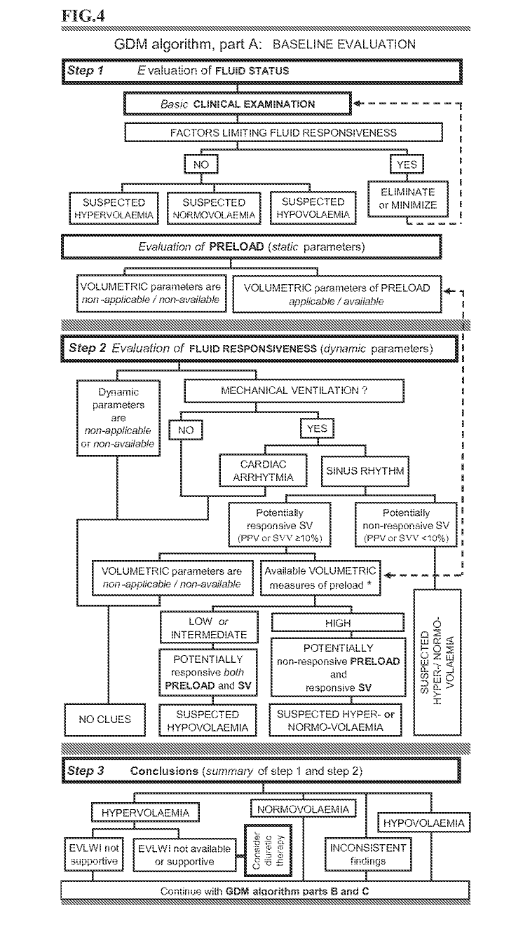

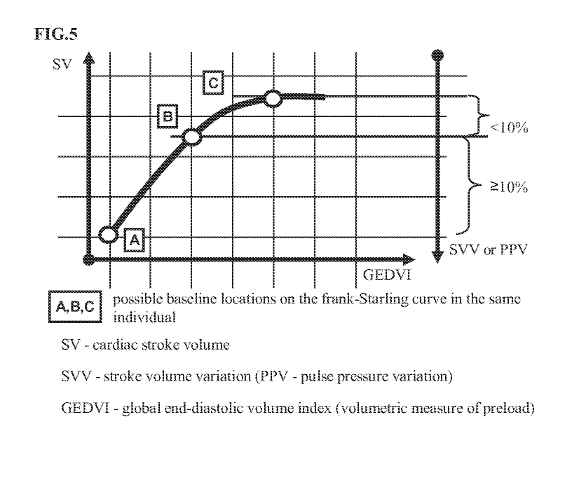

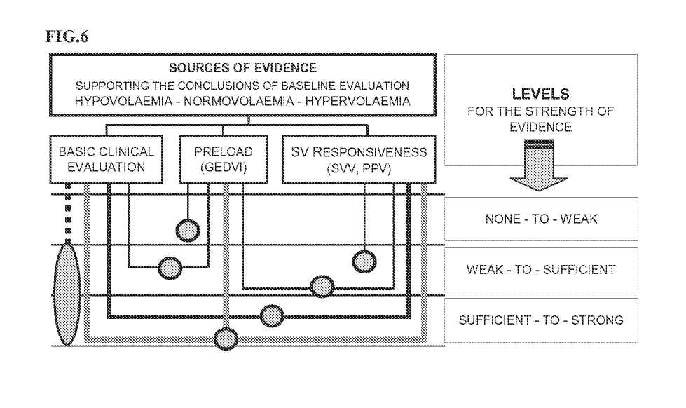

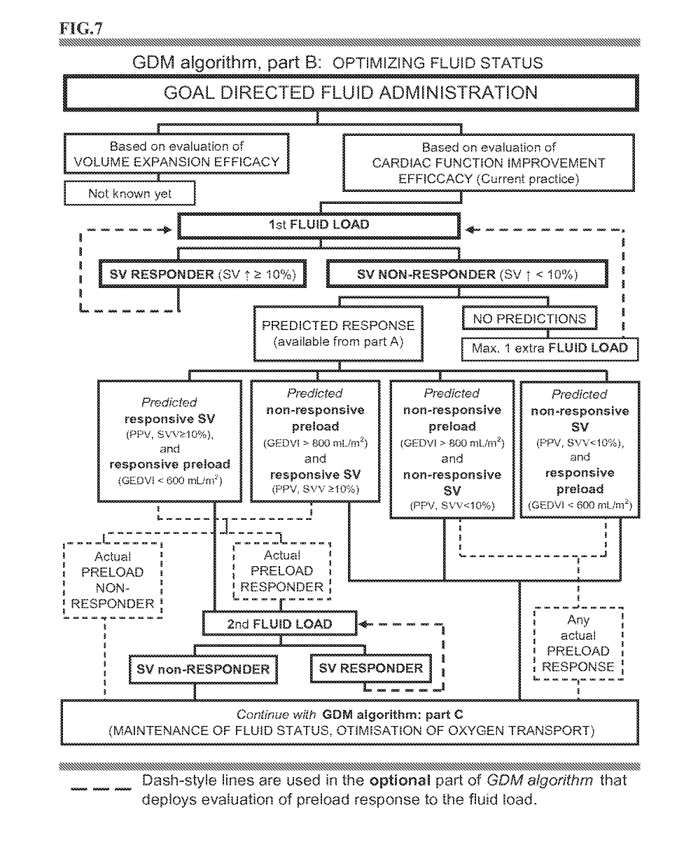

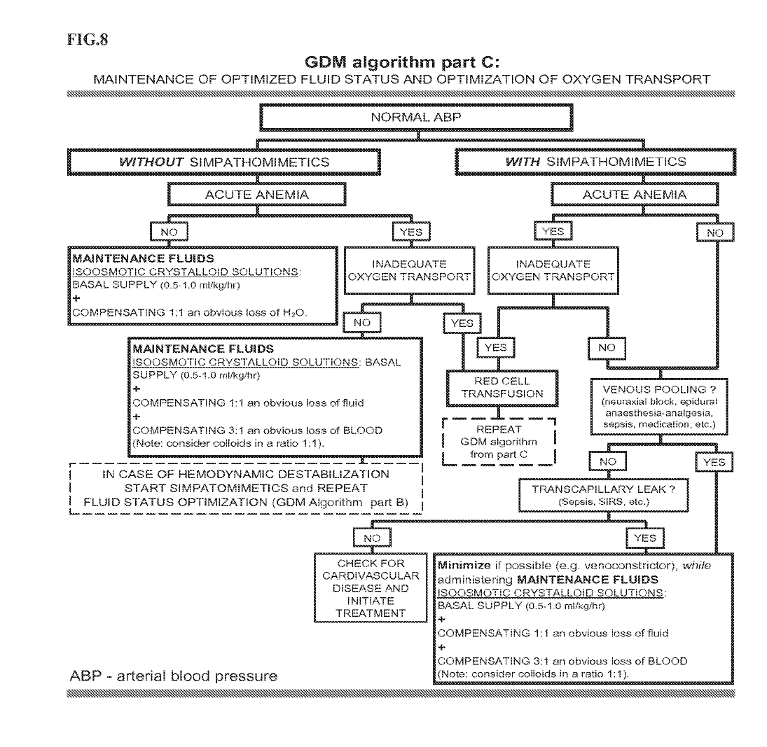

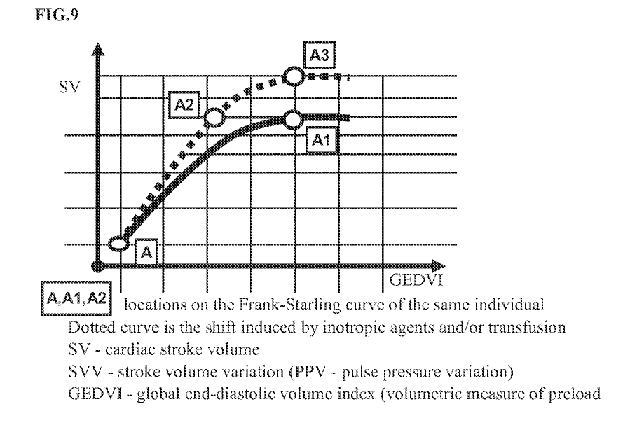

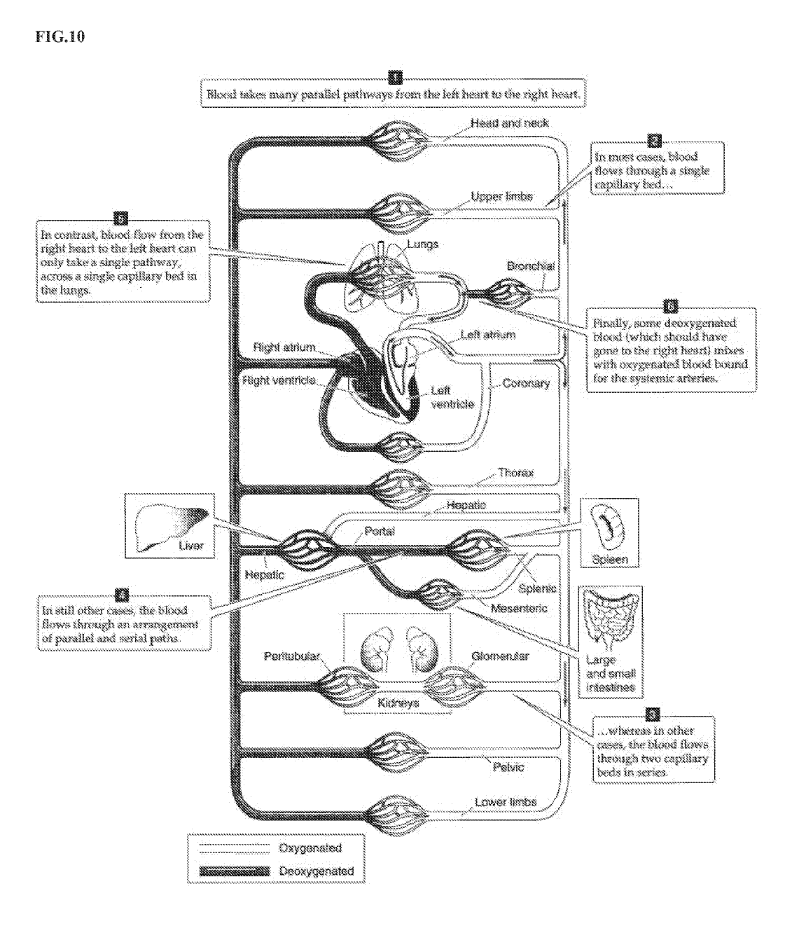

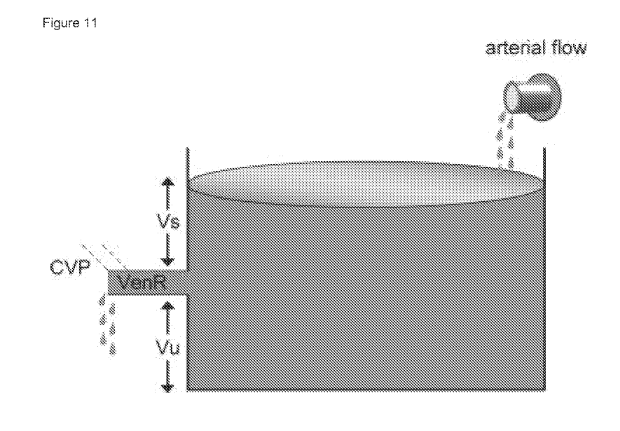

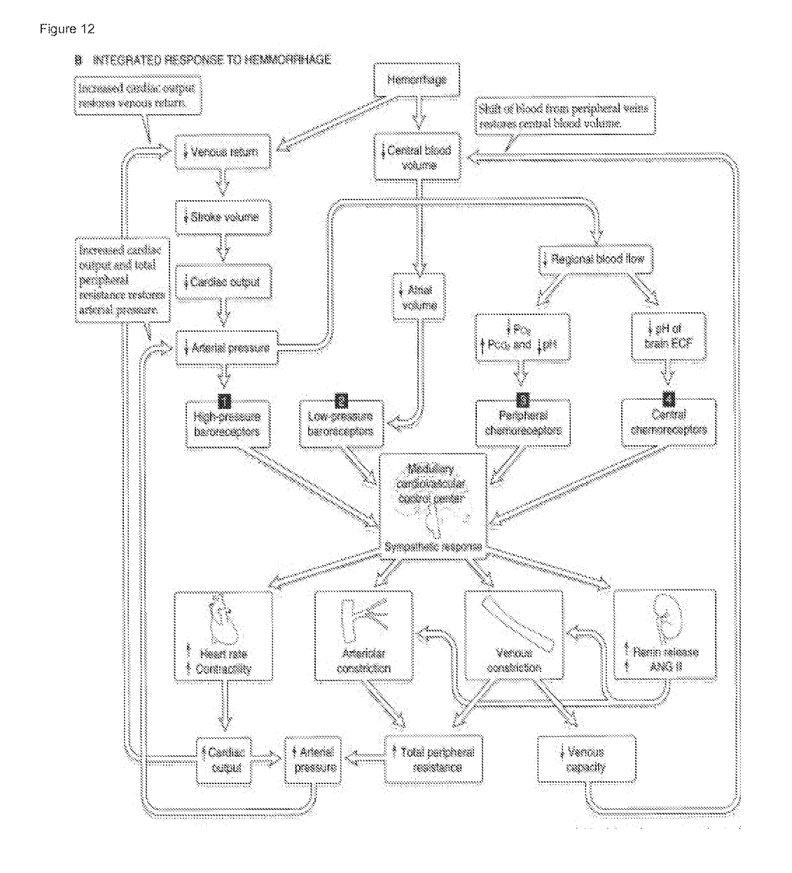

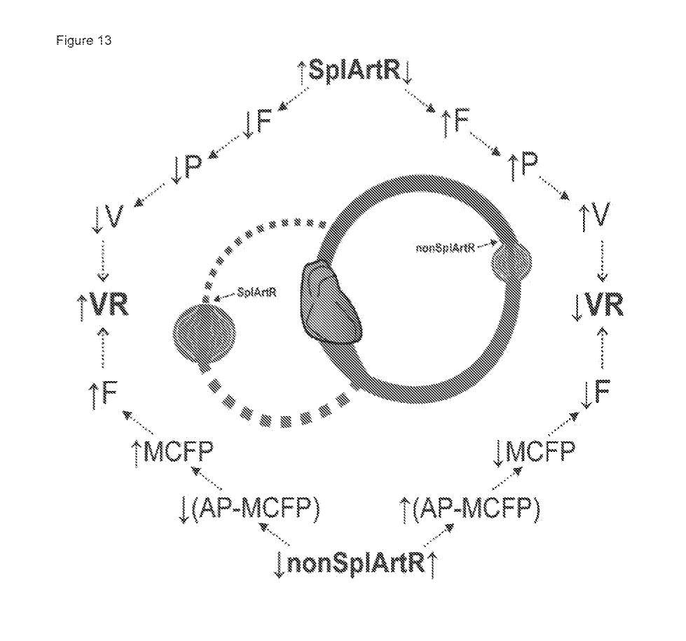

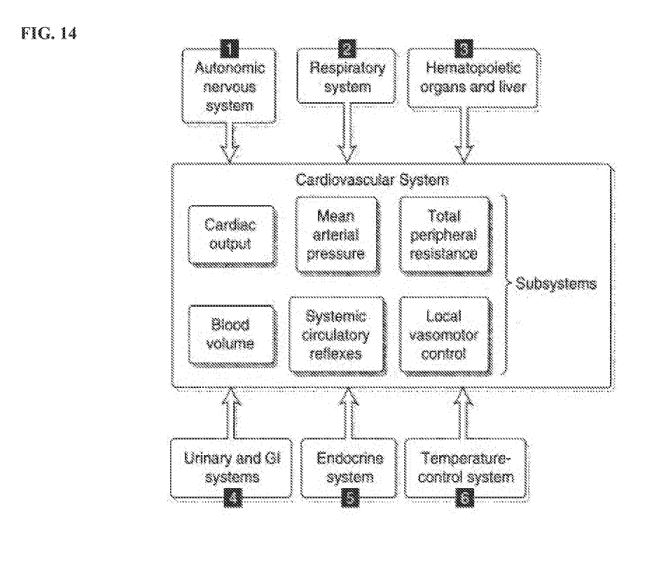

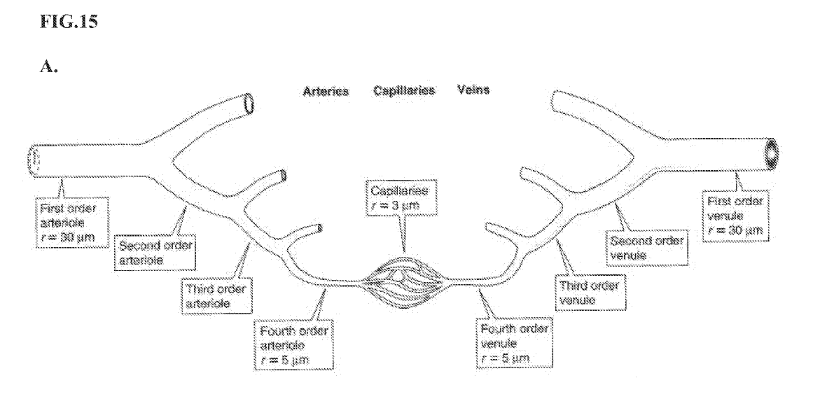

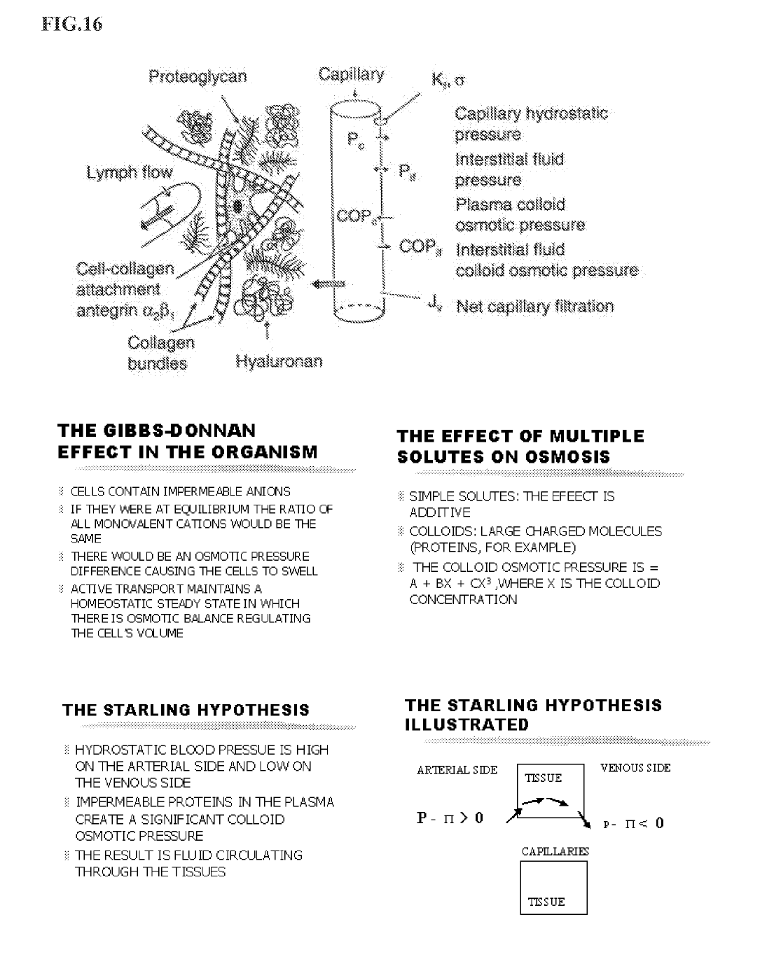

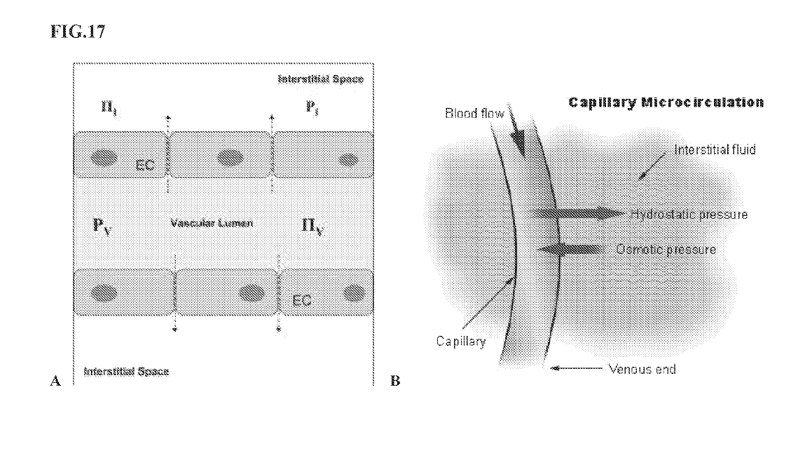

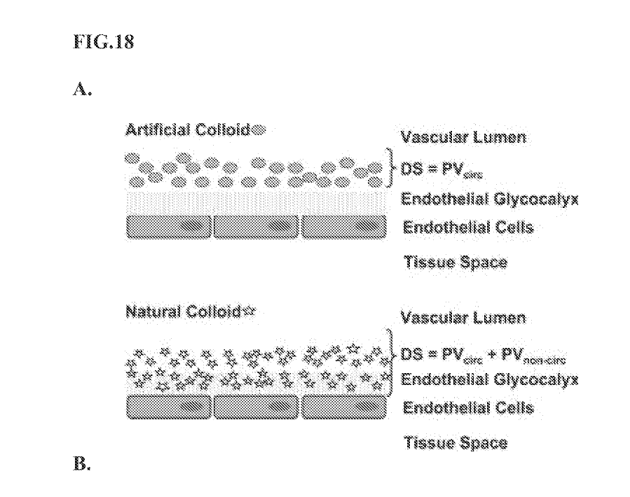

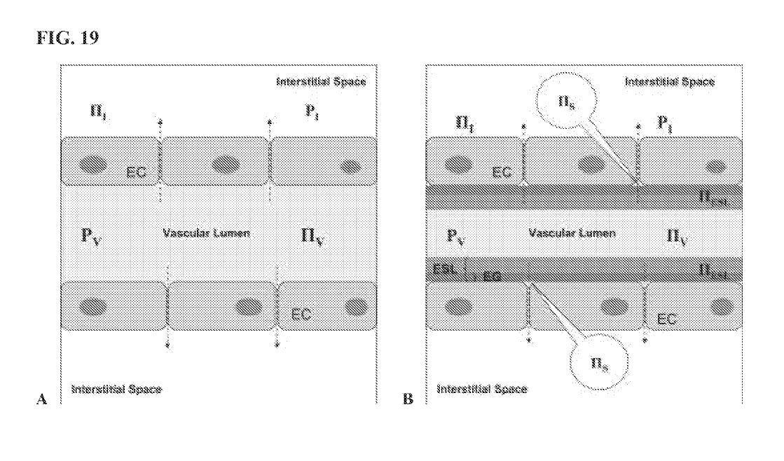

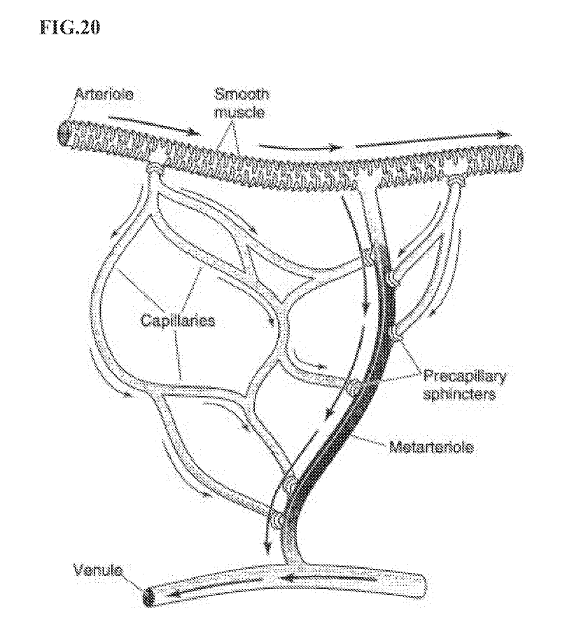

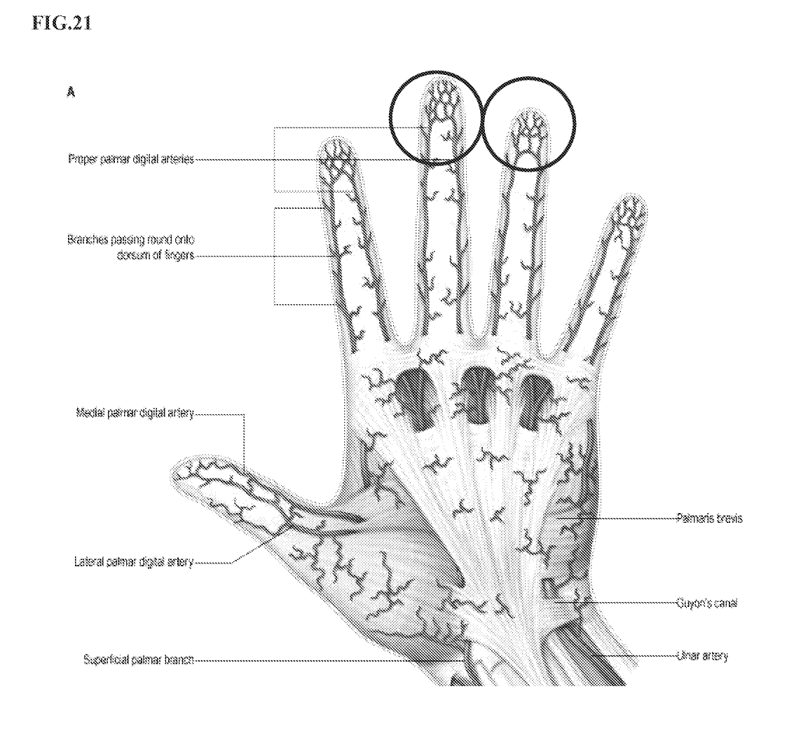

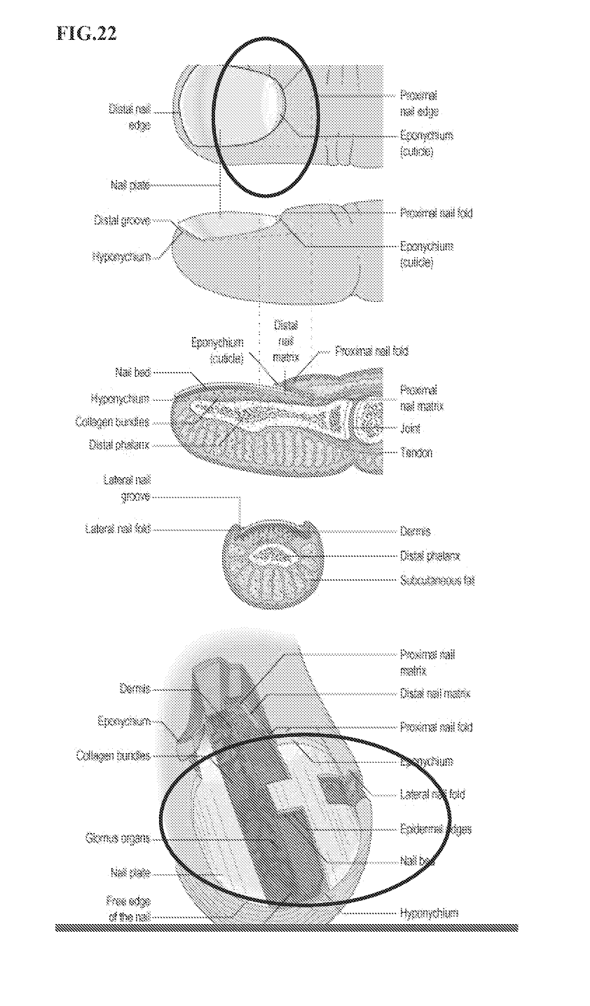

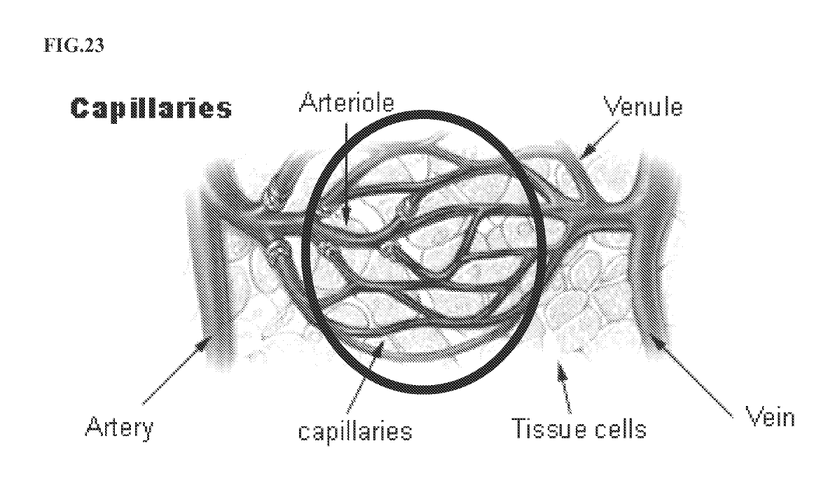

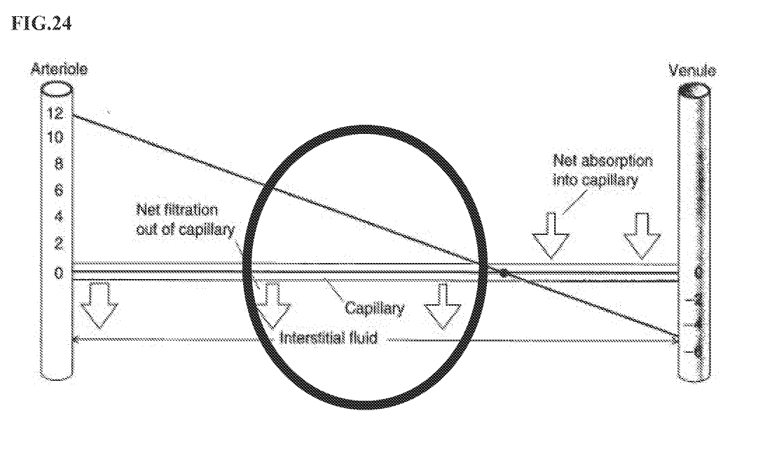

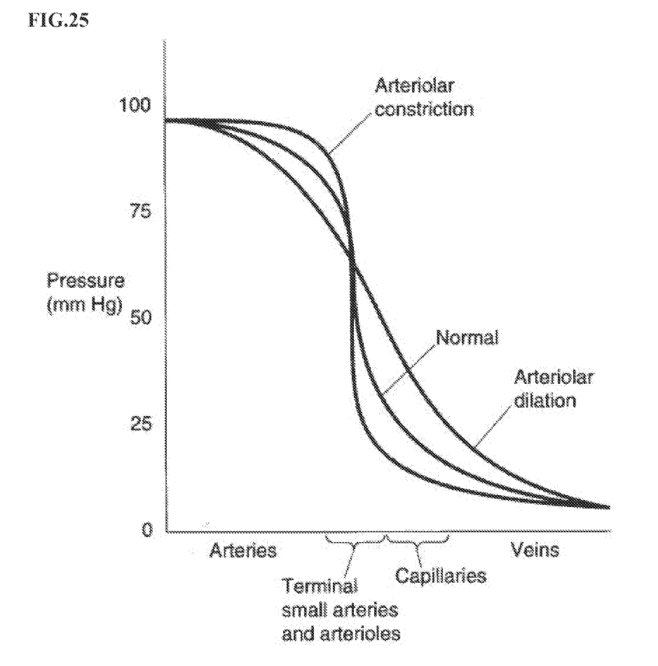

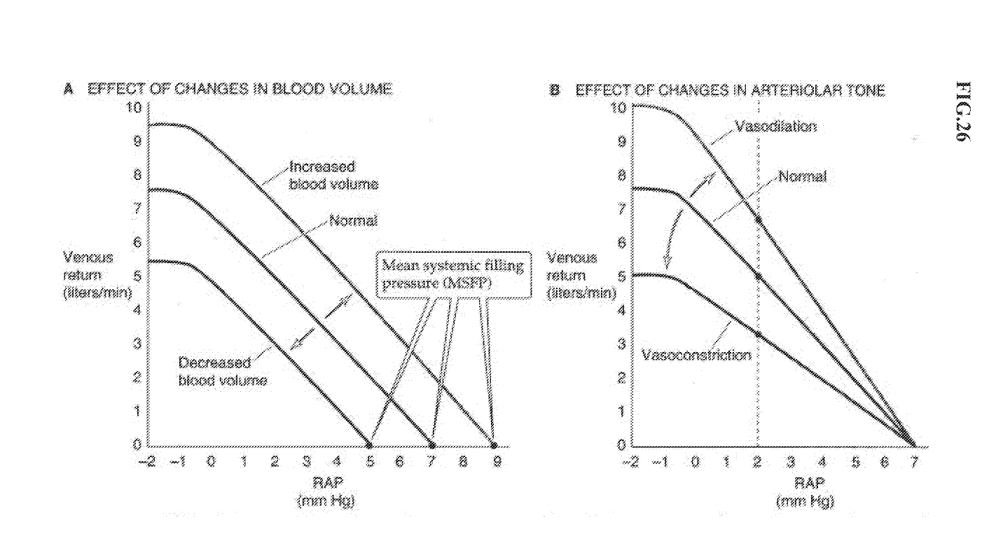

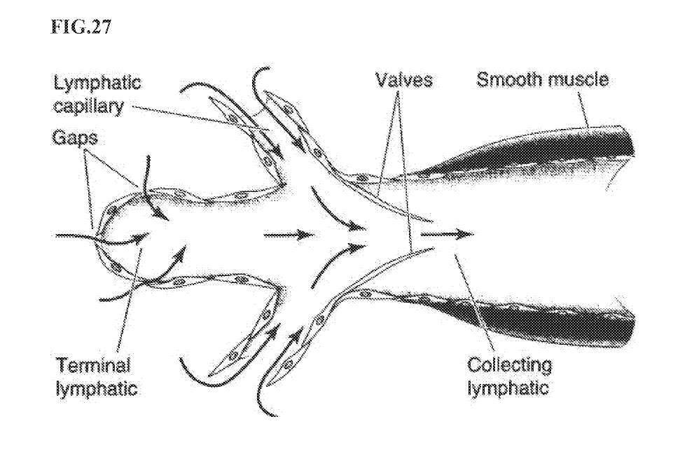

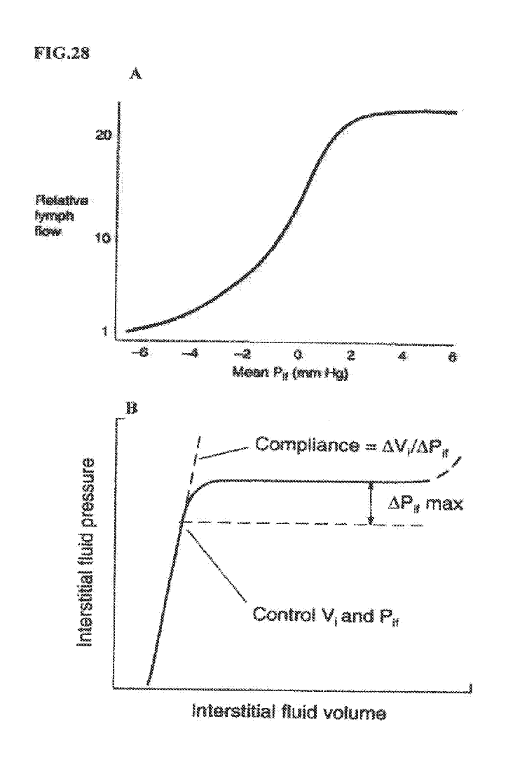

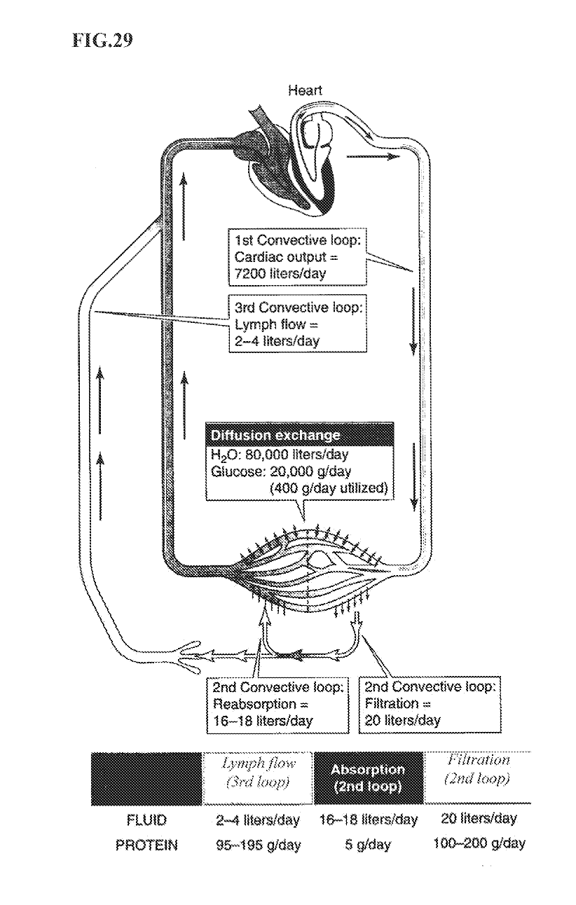

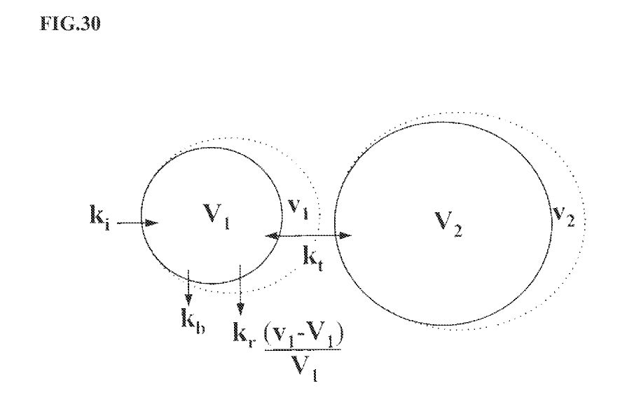

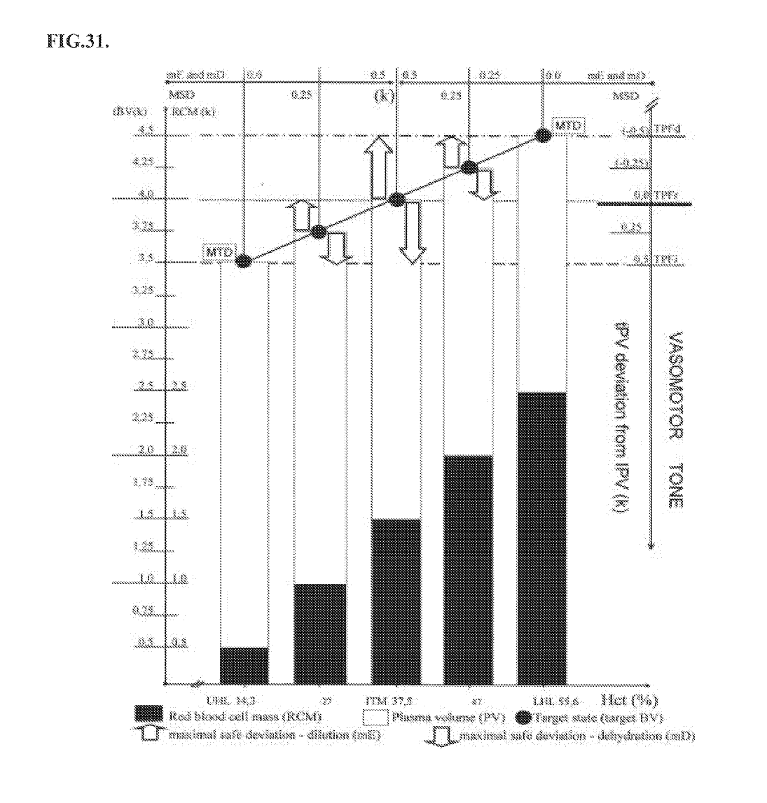

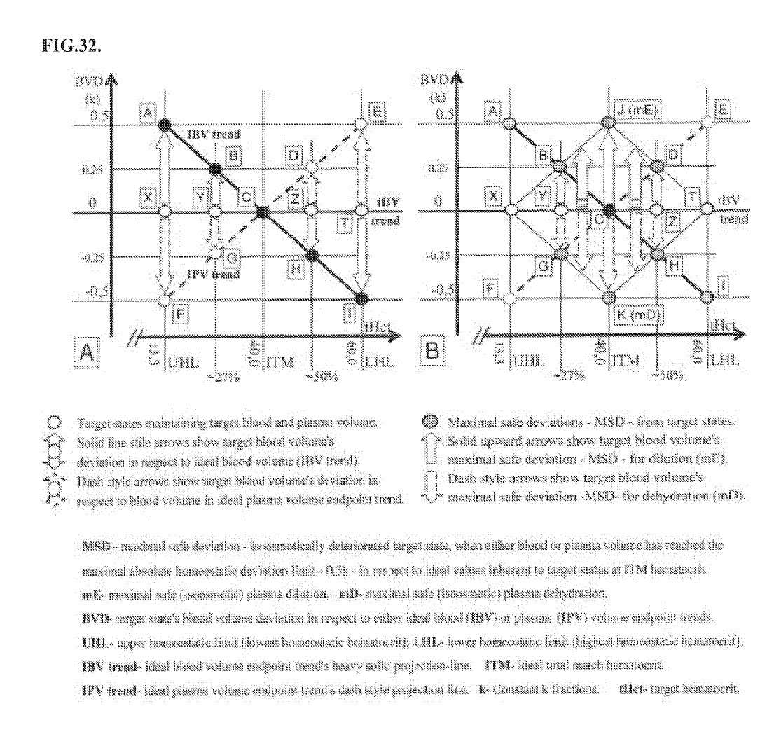

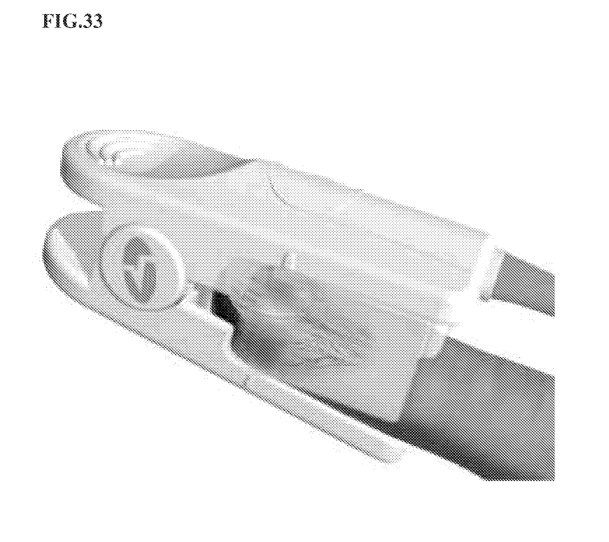

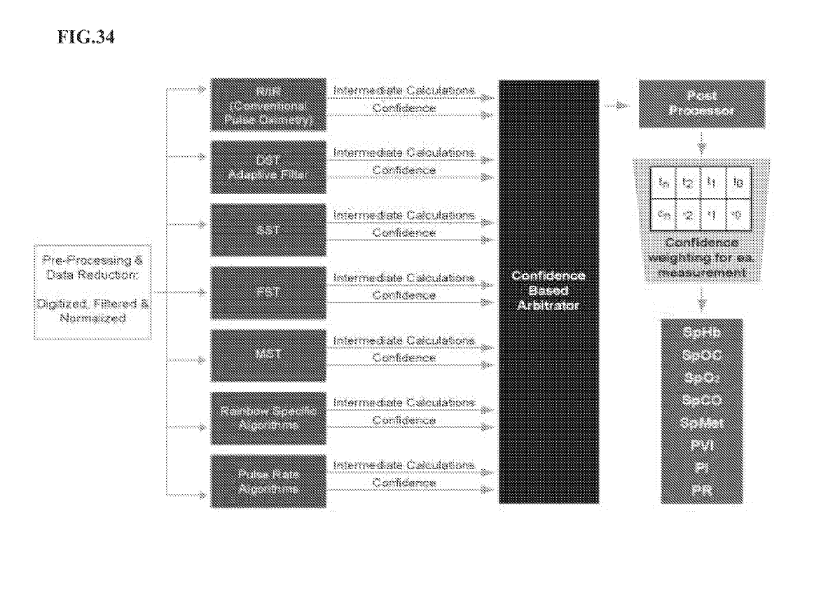

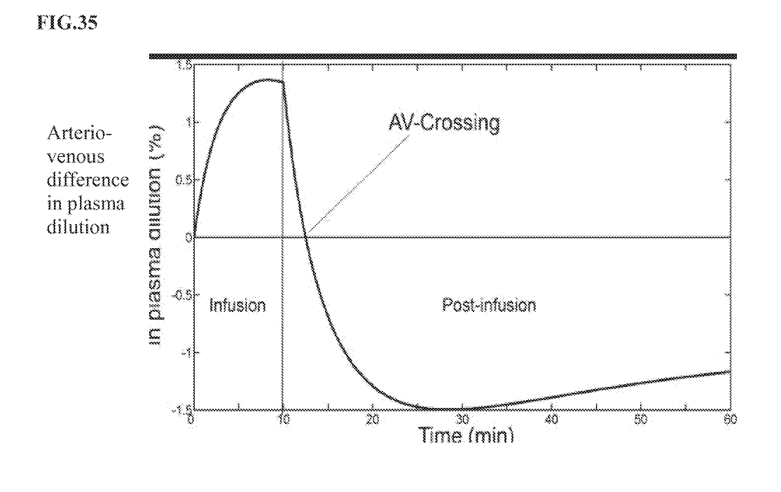

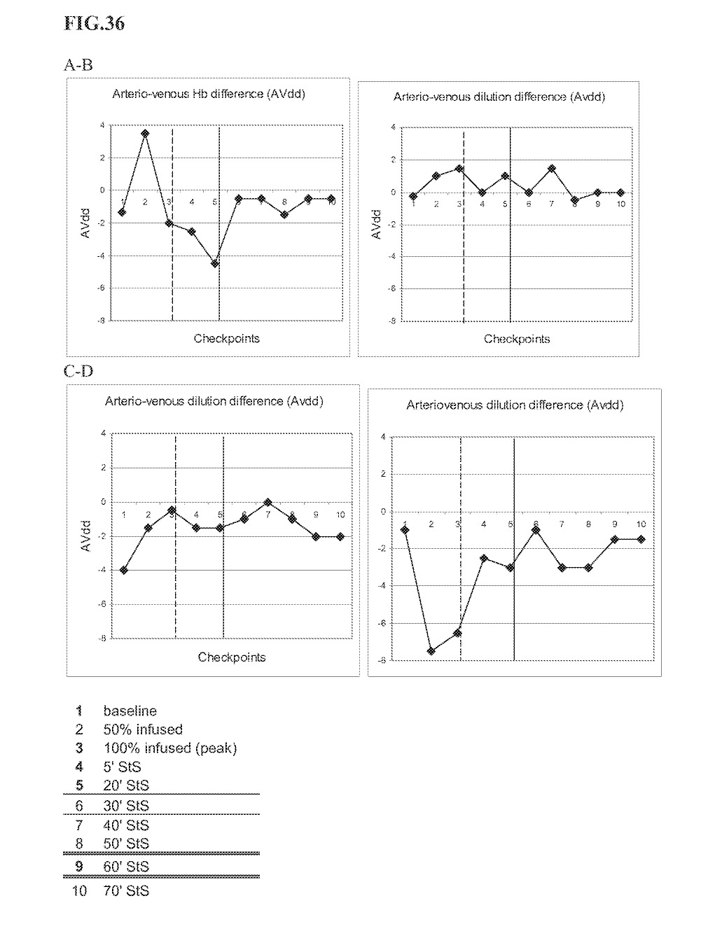

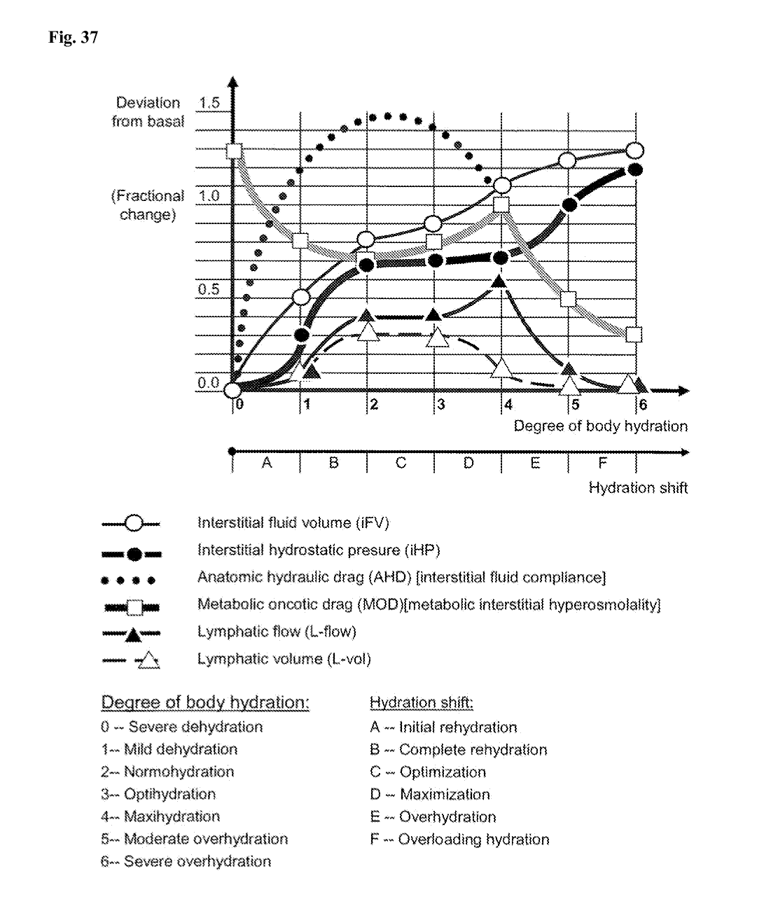

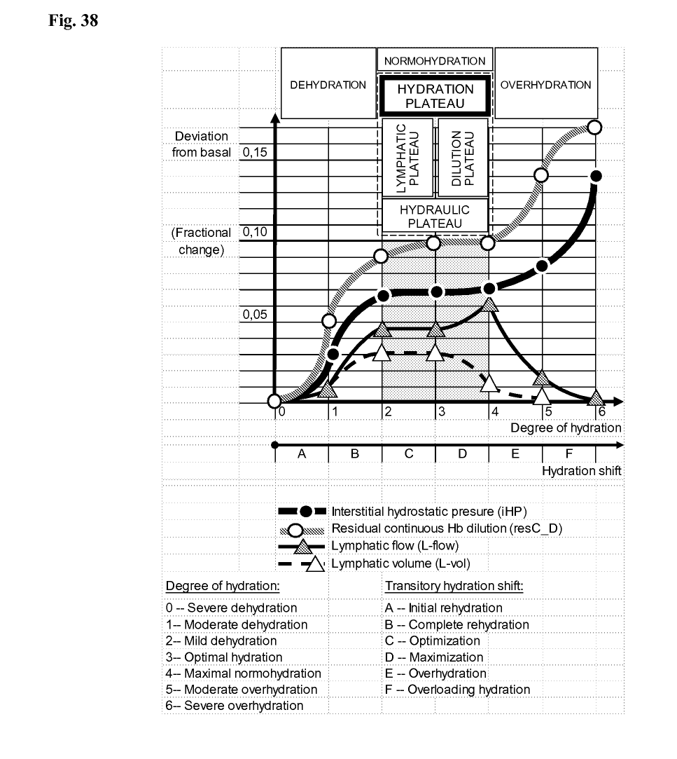

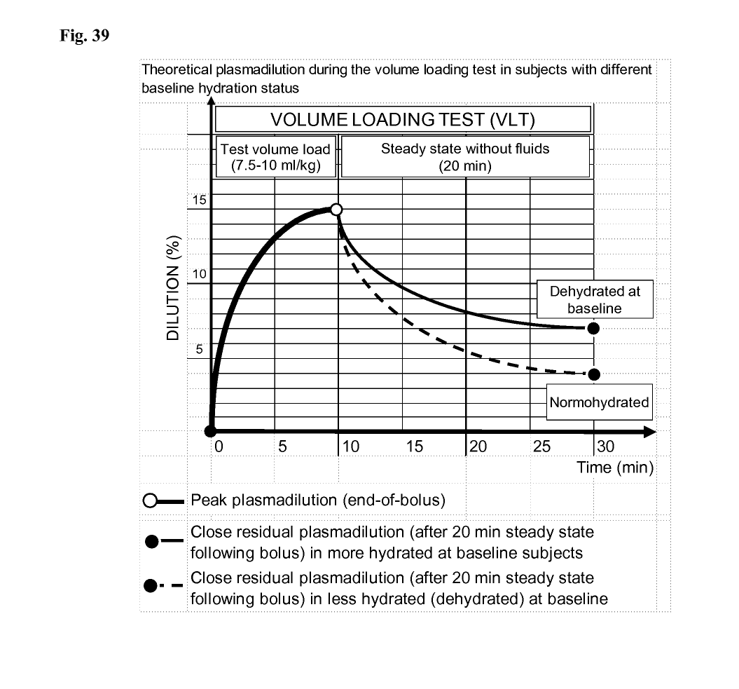

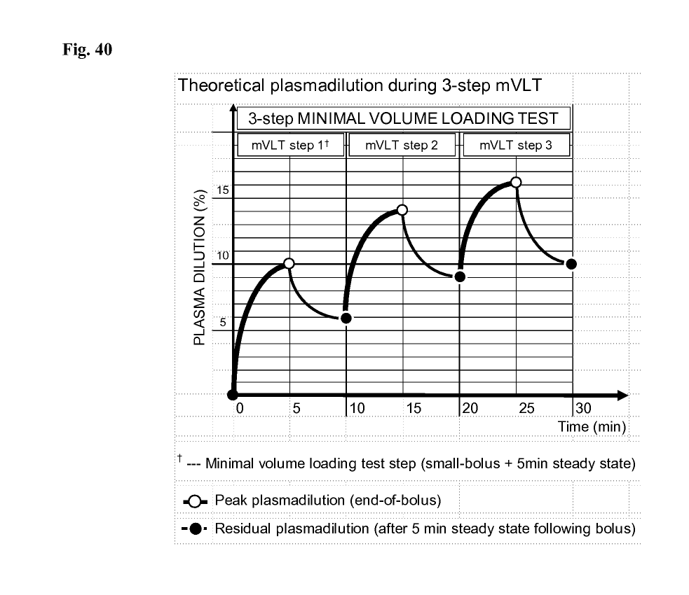

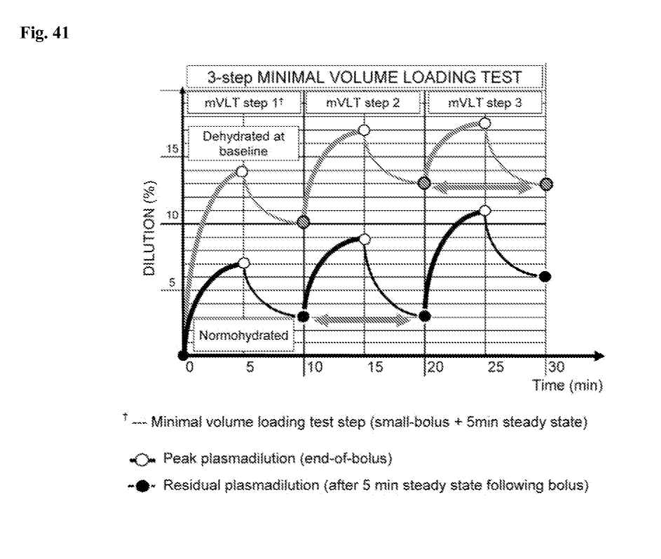

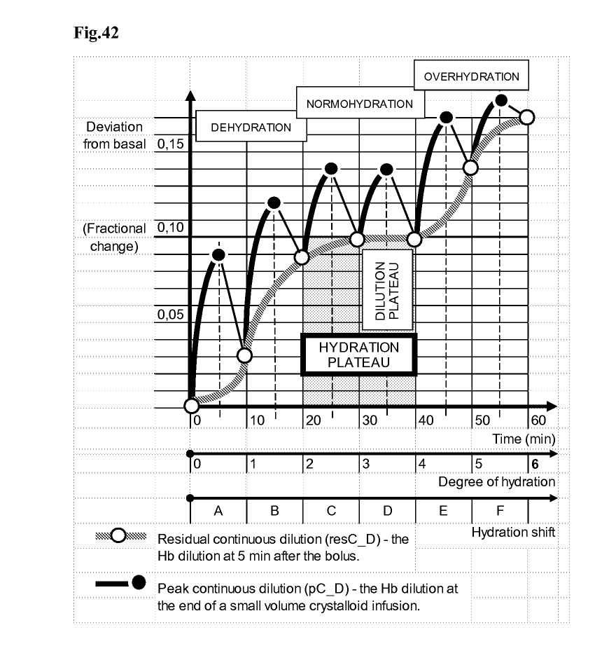

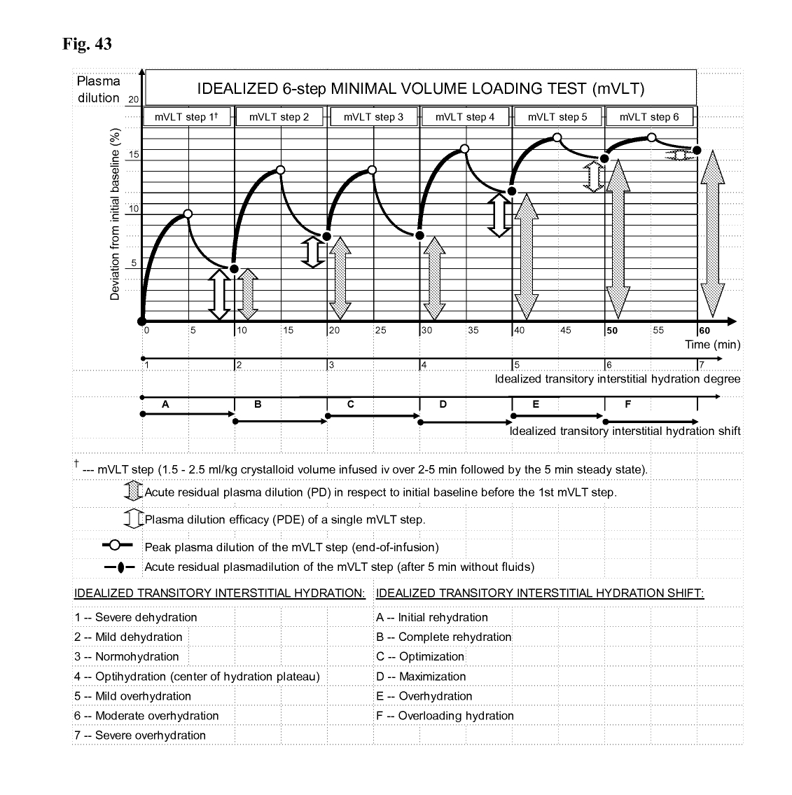

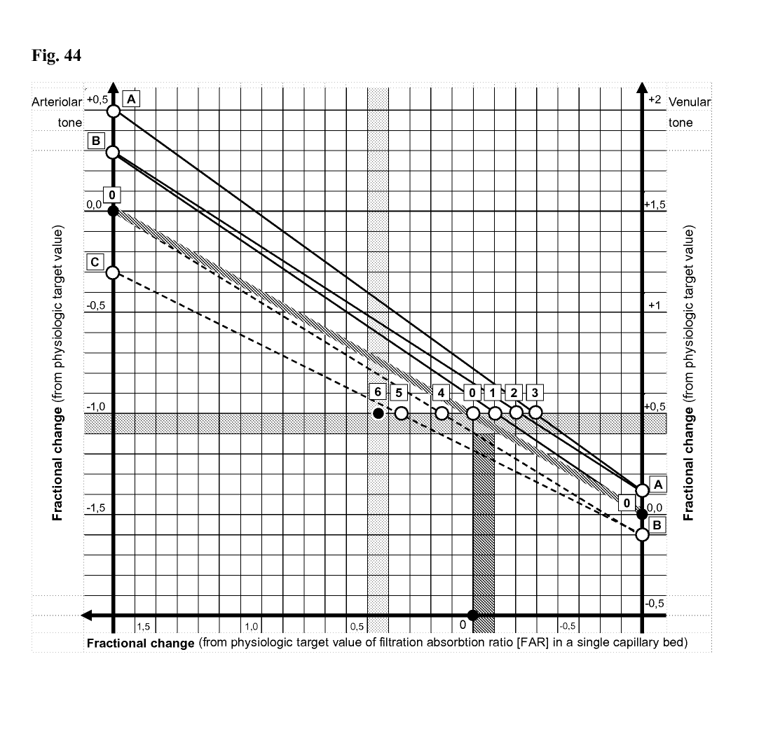

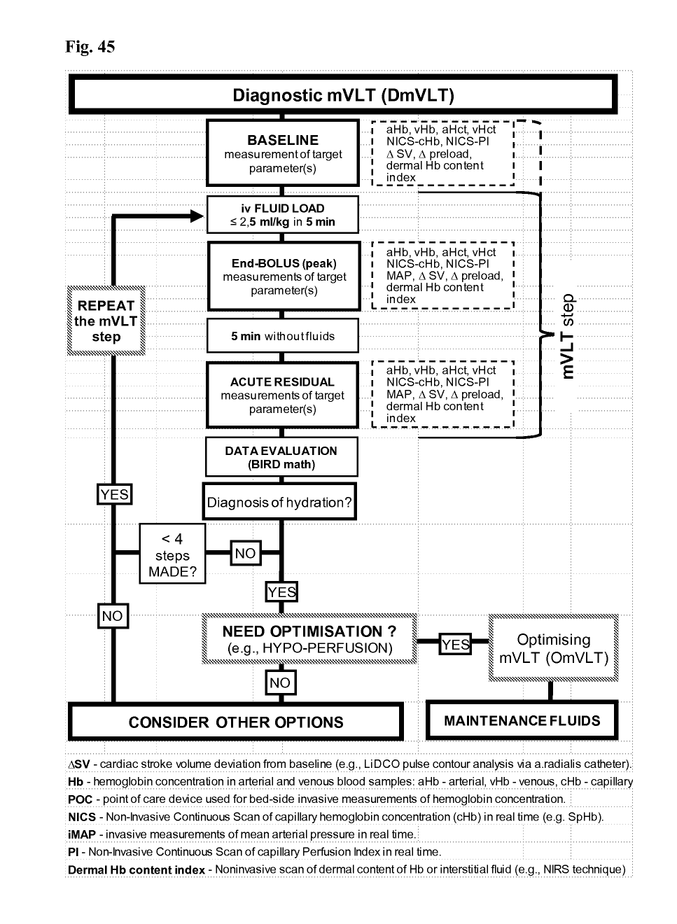

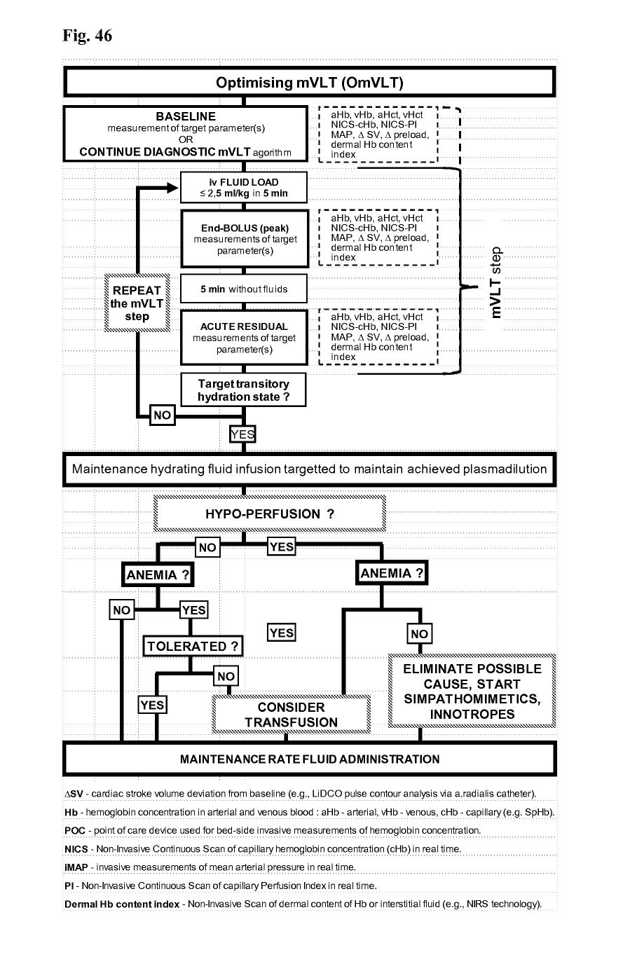

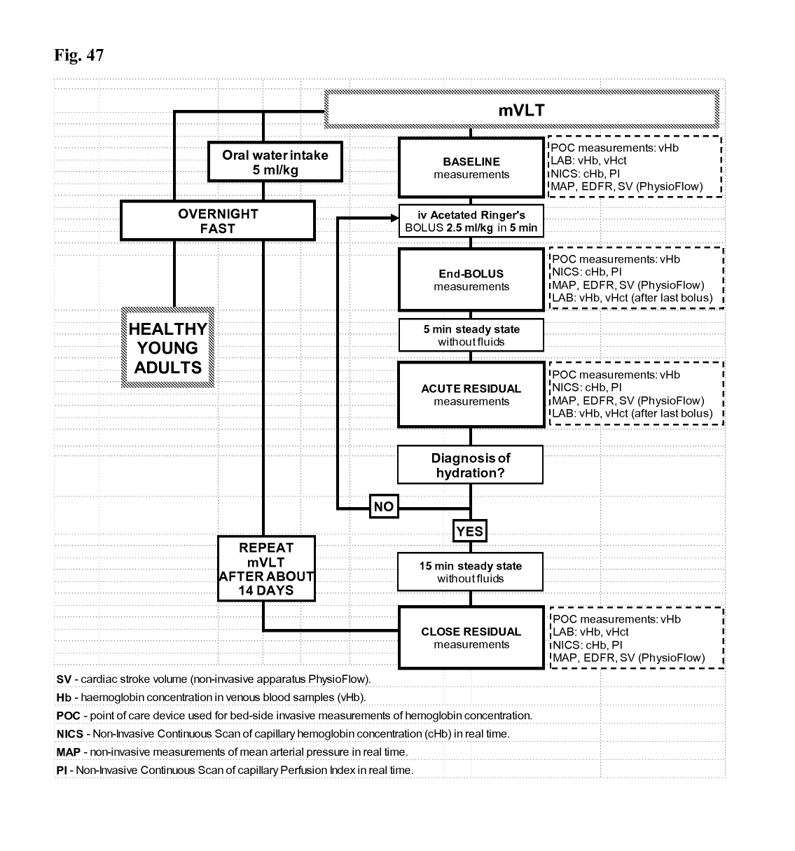

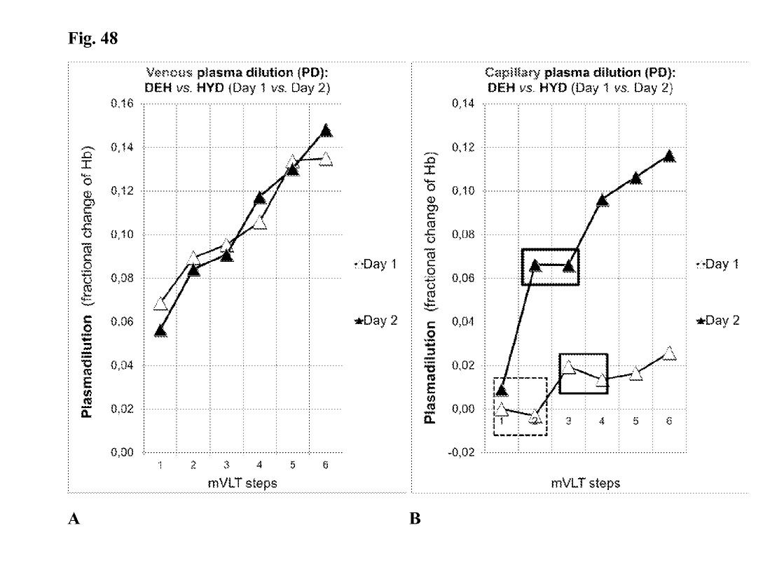

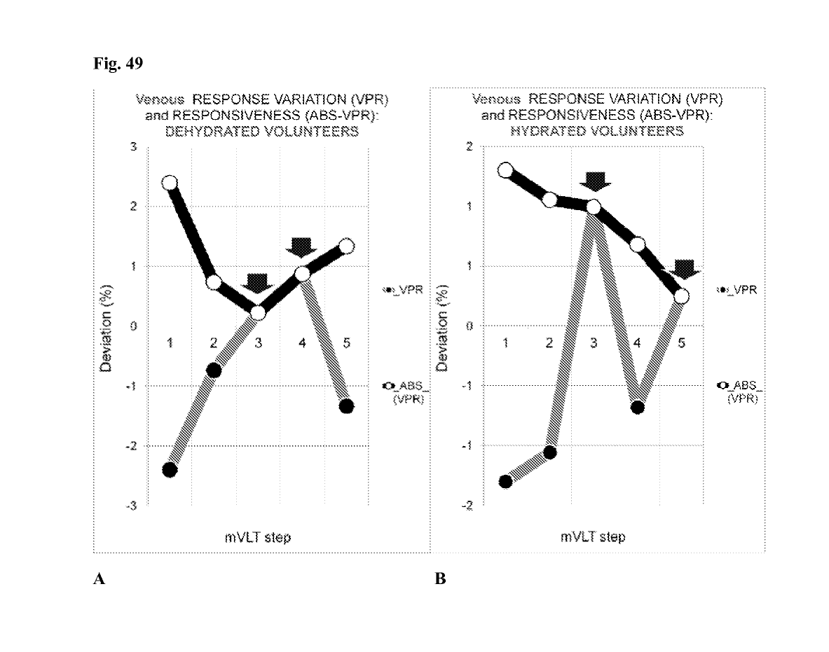

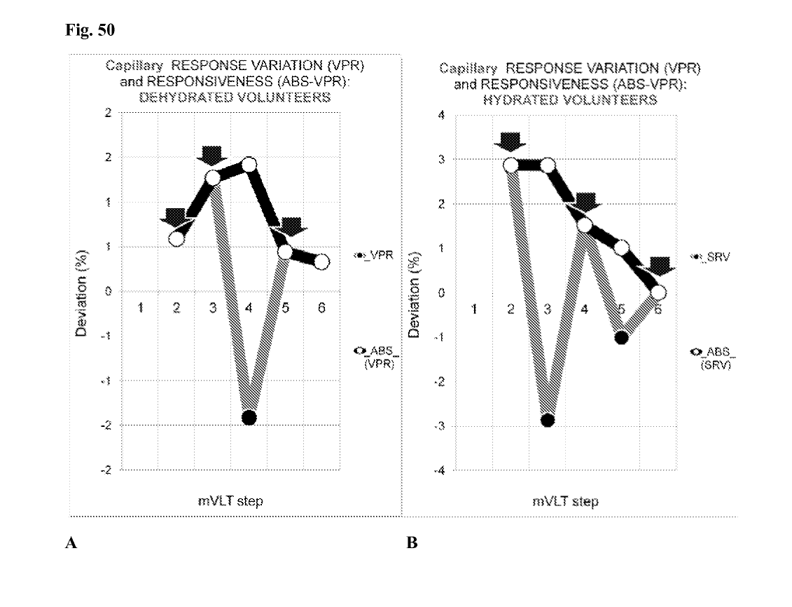

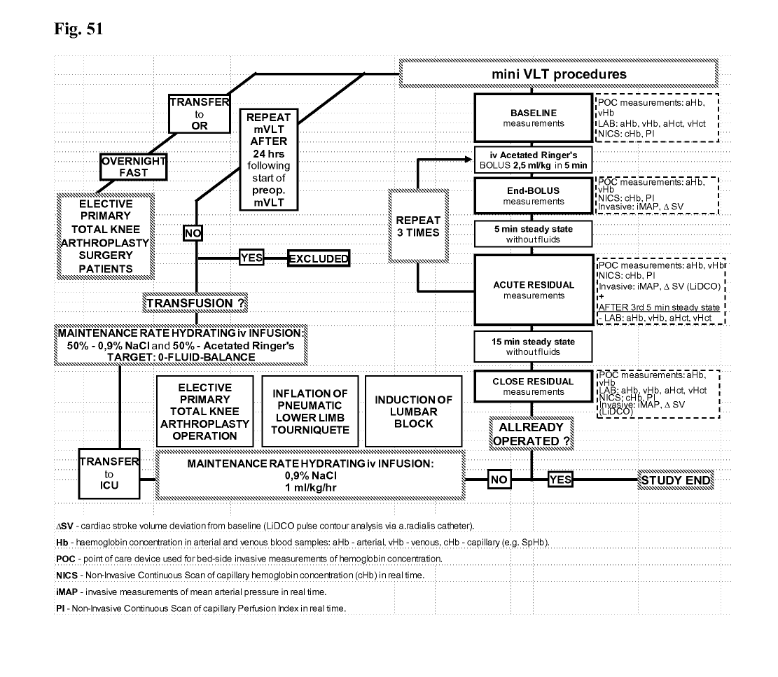

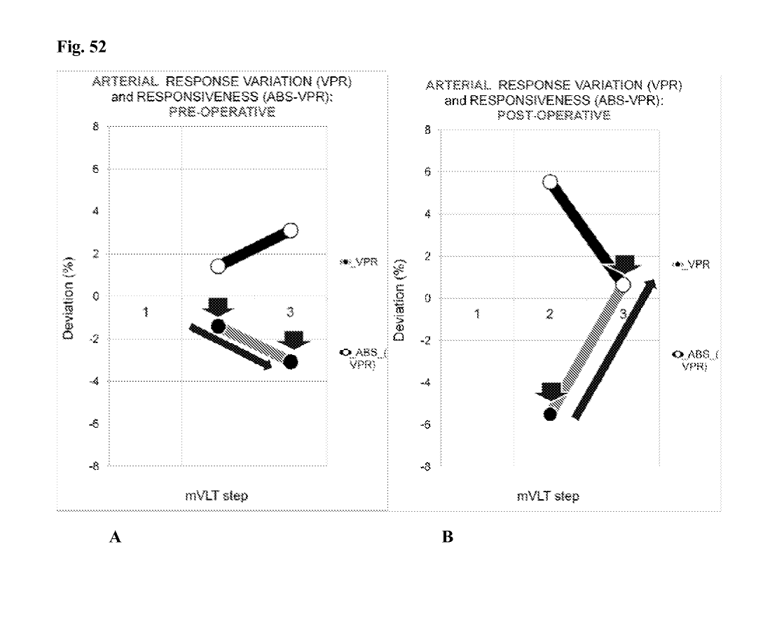

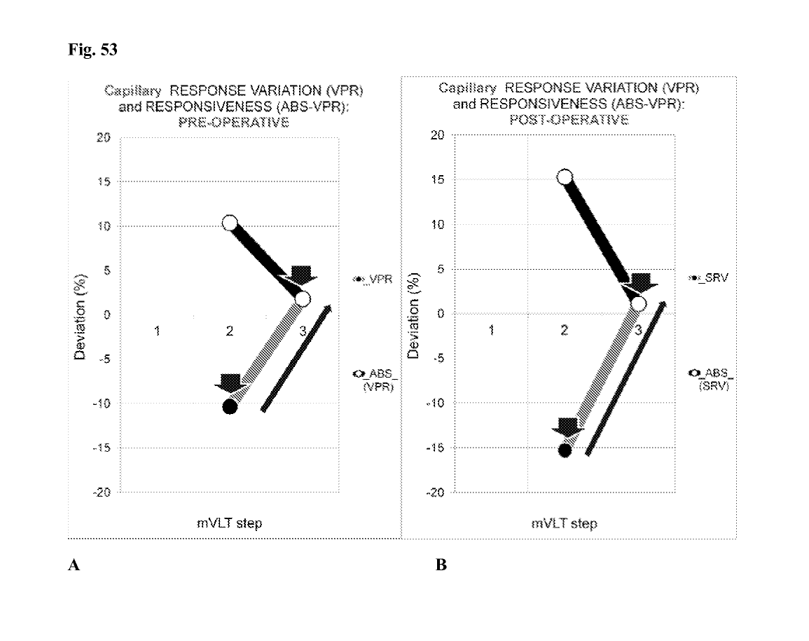

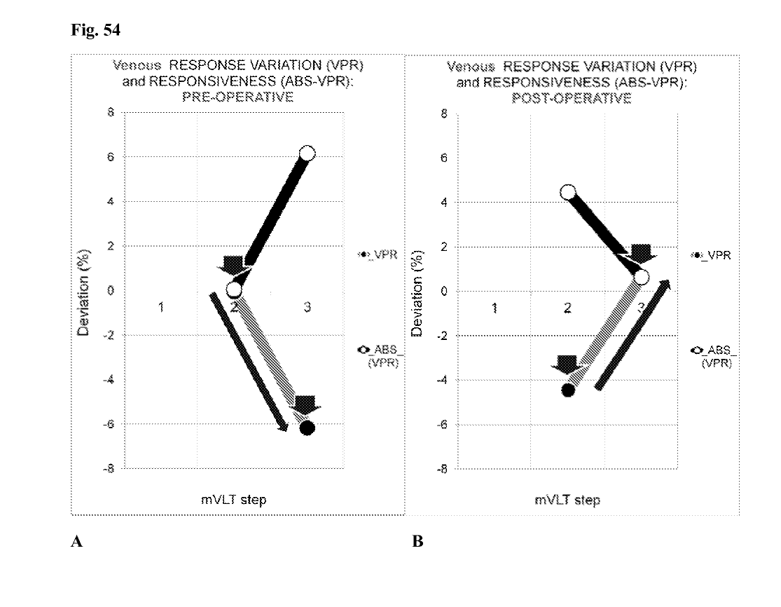

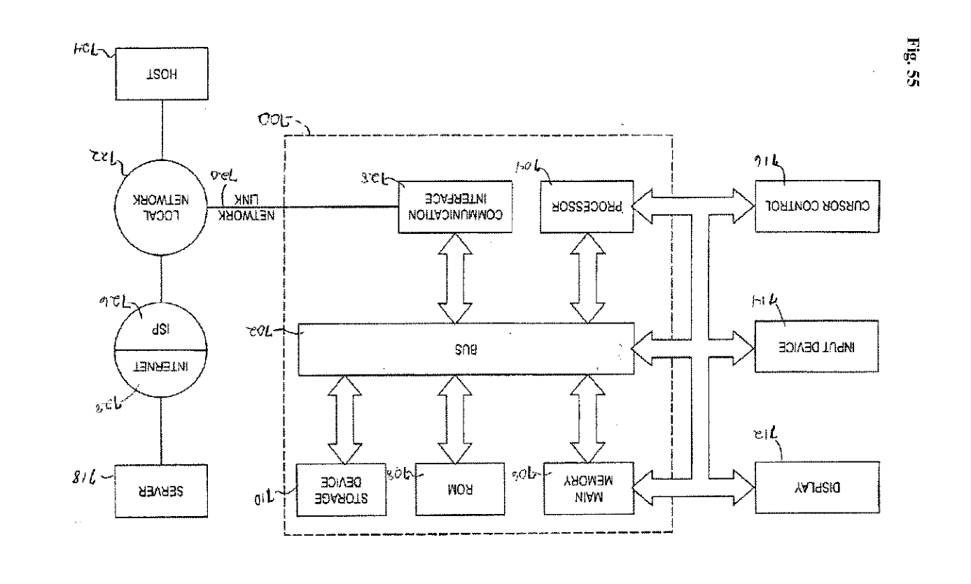

A. The “classic” Starling's principle.

B. The “revised” Starling's principle. The hydrostatic pressure in the vascular lumen (PV), which largely exceeds the interstitial pressure (PI), forces fluid outward. The endothelial glycocalyx (EG) binds plasma proteins, forming the endothelial surface layer (ESL) with a high internal oncotic pressure. The low net flux passing through the EG (arrows) has a sparse protein concentration; the oncotic pressure underneath the EG is low. Accordingly, an inward directed oncotic pressure gradient develops just across the EG, while the proteins in the small space underneath the EG are continuously cleared toward the interstitial space via the remaining net flux. The extremely simplified illustration does not consider the venular site of the revised model, suggesting free and easy access of plasma proteins toward the interstitial space. Because the hydrostatic force is low there, this should be no problem. ΠESL—oncotic pressure within the endothelial surface layer; ΠI—oncotic pressure in the interstitial space; ΠS—oncotic pressure below the endothelial glycocalyx (subglyceal); ΠV—oncotic pressure in the vascular lumen; EC—endothelial cell. [32,33]

FIG. 20. Idealized microcirculatory circuit. [20]

FIG. 21. Anatomy of microcirculation in the derma under the nail of the palm. Typical sites (encircled) for the monitoring of microcirculation related parameters.

FIG. 22. Anatomy of tissues and microcirculation under the nail of the palm. Typical sites (encircled) for the monitoring of microcirculation related parameters.

FIG. 23. The most likely part of microcirculation that can be observed by monitoring of microcirculation under the nail of the palm (encircled).

FIG. 24. The net filtration pressure in the capillary exchange of water. The net driving force in the Starling equation is the net filtration pressure. The capillary exchange of water is governed by the convective fluid transfer across capillaries and depends on net hydrostatic and osmotic forces such as Starling forces. The two driving forces for the convection of fluid across the capillary wall are the transcapillary hydrostatic pressure difference and oncotic pressure difference. The hydrostatic pressure difference is the difference between the intravascular and extravascular pressure. Filtration of fluid from the capillary occurs when net filtration pressure is positive, meanwhile reabsorbtion of fluid from interstitium occurs when it is negative. [20] Encircled is the most likely pattern of fluid flux (extravasation) in accordance with the most likely site of microcirculation that can be observed by monitoring of microcirculation under the nail of the palm (FIGS. 21-23).

FIG. 25. The arterial, arteriolar, capillary, venular and venous hydraulic pressure profiles during vasomotion. [20]

FIG. 26. The effect of changes in blood volume and arteriolar tone on venous return and right atrial pressure. A. The effect of changes in blood volume on venous return and right atrial pressure (RAP). B. The effect of changes in arteriolar tone on venous return and right atrial pressure (RAP). [20]

FIG. 27. The schematic organization of the lymphatic flow. [20]

FIG. 28. A. The dependence of lymph flow on the hydraulic interstitial pressure. [20] B. The dependence of interstitial hydraulic pressure and interstitial fluid volume (Wiig et al. Acta Anesth Scand, 2003)

FIG. 29. Convective loop of extracellular fluid and protein. [20]

FIG. 30. Fluid infused at the rate ki and is distributed in the volume V1 which is expanded to v1, and its dilution is given by (v1−V1)/V1. Elimination occurs by a dilution-dependent mechanism, kr, and a zero-order function, kb, corresponding to evaporation and basal diuresis). The fluid equilibrates with a peripheral body space having the basal volume V2, which expands to v2. The equilibration of V1 and V2 is depends on their difference in dilution and a constant kt. [44]

FIG. 31. The Homeostatic Blood Volume and Hematocrit Limits model (HHL). Target states specific red cell mass (RCM), blood volume (tBV) and corresponding plasma volume deviations from ideal value. Limits (mE and mD) of maximal safe (iso-osmotic) deviations (MSD) from target states decrease to both directions from Hct of Ideal Total Match (ITM). Any safe deviations are homeostatically allowed at critical Hct limits—UHL and LHL—as MSD states reach the value of maximal target deviation (MTD) equal to 0.5 k (k—is for Constant k, which is equal to 0.25·IBV, if assumed ITM-Hct is equal to 37.5% and assumed UHL-Hct is 14.4%). Vasomotor tone is adjusting to maintain adequate or target tissue perfusion consistent with effective circulating volume fitting the different patterns of target blood volume: TPFd—target perfusion focused decreased tone, TPFd—resting and TPFi increased. [27-28]

FIG. 32. The Homeostatic Hematocrit Limits model (HHL).

A—Maximal homeostatic deviations from ideal blood volume are applicable to different homeostatic target blood volume with different red cell amount in circulation; limits inherent to target blood volume states at different Hct values along the physiologic range. B—Limits (E,D) of safe (iso-osmotic) maximal homeostatic fluctuations of blood volume that can be induced by changes in plasma fluid content; limits inherent to target blood volume states at different Hct values along the physiologic range. [27-28]

FIG. 33. Total hemoglobin (SpHb™) is measured noninvasively under the nail of the hand by a range of probes. [54]

FIG. 34. Operation of the Masimo Rainbow SET® Pulse CO-Oximetry™ device that provides total hemoglobin (SpHb™) is based on Masimo SET Pulse Oximetry Technology with added Rainbow Technology Algorithms. [54]

FIG. 35. Arterio-venous (AV) difference in plasma dilution in the forearm of 15 volunteers receiving 15 mL/kg of lactated Ringer's solution over 10 min. The smoothened curve represents the mean of all calculated curves. [49]

FIG. 36. Individual charts of arteriovenous difference (AVdd) in plasma dilution in 4 volunteers who received 15 mL/kg of lactated Ringer's solution over 10 min. Negative values indicate that arterial plasma dilution was exceeding venous (arterial Hb was lower than venous). Charts made by the present inventor using the data provided by the main investigator of the 15 volunteer study. [49]

FIG. 37 depicts the theoretical parallel fractional changes of related parameters/variables during changes of interstitial hydration degree in a single capillary-lymphatic bed during the idealized 6-step mVLT.

FIG. 38 depicts the theoretical corresponding fractional changes of different parameters during mVLT induced changes of interstitial hydration degree in the context of the hydration plateau. The latter is formed by the lymphatic plateau, the hydraulic plateau and the dilution plateau. The lymphatic plateau is when lymphatic flow does not increase in two mVLT steps despite increase of interstitial fluid volume (also see FIGS. 28 and 41), also the lymphatic volume and hydrostatic pressure do not increase either. At that point the maximal sum tissue fluid compliance is reached. The dilution plateau manifests in the next shift of hydration when plasmadilution of the mVLT step (PD or resC_D) does not increase despite starting to increase interstitial tissue fluid compliance (also see FIG. 41). That is because interstitial hydrostatic pressure does not rise since evacuation of the lymphatic vessels frees some space for interstitial fluid accumulation.

FIG. 39 depicts the background concept of the volume loading test (VLT) method. The plasma dilution values (dimensionless, in percent) during a theoretical one-step volume loading session. Onerrelatively big bolus (7.5-10 ml/kg) of acetated Ringer's solution is given. It is followed by a 30 min steady state period when no fluid is given. Presumably, the better hydrated individuals will present with a more pronounced ‘close residual’ dilution than less hydrated subjects.

FIG. 40 depicts plasma dilution values (dimensionless, in percent) during a theoretical three-step minimal volume loading test. Three 1.5-2.5 ml kg⁻¹ boluses of acetated Ringer's solution are infused over a short time up to 5 min long. Each bolus is followed by a 5 min steady state period when no fluid was given. Peak points are at 5, 15 and 25 minutes, but plasma dilution is not interpreted by the mVLT. Residual or total plasmadilution (PD) is defined as plasma dilution value at time point 10, 20 and 30 minutes in respect to initial baseline at time point 0 minutes.

FIG. 41 depicts the dilution values (dimensionless, in percent) during a theoretical three-step minimal volume loading test (3-step mVLT). The figure shows two hypothetical initial baseline states of body hydration—hydrated and dehydrated. A dilution plateau is reached when two residual or total plasma dilution (PD) values are equal (values connected by the bidirectional horizontal arrows). Presumably, the better hydrated individuals will reach this plateau earlier than less hydrated subjects.

FIG. 42 depicts the theoretical plasmadilution course during the theoretical six-step minimal volume loading test (6-step mVLT). A dilution plateau which is part of the hydration plateau is reached when two residual or total plasma dilution (PD) values are equal (hydration shift D). The peak dilution is considered of no value for the detection of the hydration plateau since it is affected by the numerous factors other than interstitial tissue fluid compliance.

FIG. 43 depicts the total or acute residual plasma dilution (PD, dimensionless, in percent) during a theoretical six-step minimal volume loading test (6-step mVLT). Six relatively small test boluses (1.5-2.5 ml/kg) of acetated Ringer's solution are infused over up to 5 minutes period. Each step is followed by a 5 min steady state when no fluid is given. Peak points (end of test bolus) are not considered by the mVLT. The total plasma dilution (PD) is defined in respect to initial baseline at time point 0 (PD is illustrated by the dark bidirectional arrows). Plasma dilution efficacy (PDE) is the shift of total plasma dilution (PDD) in a single mVLT step (illustrated by the white bidirectional arrows). It decreases on approach to the dilution plateau where it is minimal due to the maximal interstitial fluid compliance, and steeply increases after it.

FIG. 44 depicts the theoretic corresponding shifts of filtration absorption ratio (FAR) and arteriolar and venular tone in relation with changes of interstitial hydration degree where 0 (in a frame) is the normal hydration, the 3 is the severe dehydration, and 5 is the overloading hydration. Data point 6 is the state when midcapillary FAR is zero (also see TAB.2).

FIG. 45. Depicts the Diagnostic mVLT (DmVLT) which consists of at least four mVLT steps.

FIG. 46. Depicts optimizing mVLT (OmVLT) which consists of more than four mVLT steps.

FIG. 47 depicts the protocol of the randomized crossover healthy young volunteer study for the investigation of mVLT capability to discriminate between hydrated and dehydrated subjects.

FIG. 48 depicts the results of randomized crossover study in 5 healthy young volunteers for the investigation of the 6-step mVLT's capability to discriminate between the better hydrated (HYDRATED) and less hydrated (DEHYDRATED) subjects. The total of ten 6-step mVLTs was performed on two occasions for five healthy volunteers. The mean venous plasma dilution was similar on both occasions (A), but the capillary plasma dilution (derived from SpHb™ or cHb) was significantly more advanced in the mVLT for the better hydrated subjects (B). Markers of hydration plateau—equal capillary PD (derived from noninvasive SpHb™) in two consecutive mVLT steps or PD that is lower than the PD of the preceding step—are shown in the rectangles. Venous trends were not informative in that context. Note that the earlier appearance of capillary hydration plateau in less hydrated does not mean they were better hydrated according to the diagnostic criteria of the mVLT (TAB.3). All that matters is the trends of response variation and responsiveness as described in FIG. 49.

FIG. 49 depicts the results of randomized crossover study in 5 healthy young volunteers for the investigation of the 6-step mVLT's capability to discriminate between the better hydrated (HYDRATED) and less hydrated (DEHYDRATED) subjects. (A) In steps 3 and 4, the less hydrated subjects presented with increasing positive mean response variation (VPR) value supported by the increasing responsiveness which is the absolute value of VPR. (B) In contrast, the better hydrated volunteers presented with VPR and ABS-VPR variables decreasing from the beginning of their availablity—in a shift from step 3 to 5. According to the diagnostic criteria of the mVLT method (also see TAB. 3), these findings are consistent with the presumed better baseline hydration in the latter group. Note that trends start with the first mVLT step considering the availability of the variable (it means that step one is actually the second step in the course of mVLT).

FIG. 50 depicts the results of randomized crossover study in 5 healthy young volunteers for the investigation of the 6-step mVLT's capability to discriminate between the better hydrated (HYDRATED) and less hydrated (DEHYDRATED) subjects. (A) In steps 2 and 3, the less hydrated subjects presented with increasing positive mean response variation (VPR) value supported by the increasing responsiveness which is the absolute value of VPR. The trends began to decrease in a shift from step 3 to 5 and 6. (B) In contrast, the better hydrated volunteers presented with VPR and ABS-VPR variables decreasing from the beginning of their availablity—in a shift from step 2 to 4, and further in a shift from step 4 to 6. According to the diagnostic criteria of the mVLT method (also see TAB. 3), these findings are consistent with the presumed better baseline hydration in the latter group. Note that trends start with the first mVLT step considering the availability of the variable (it means that step one is actually the second step in the course of mVLT).

FIG. 51 depicts the protocol of the randomized clinical study in 36 TKA surgery patients, who underwent two 3-step perioperative mVLT sessions each (72 experiments), for the investigation of method's capability to discriminate between pre-operatively dehydrated (12 hr fasting) and postoperatively more hydrated (24 hrs in ICU) subjects.

FIG. 52 depicts the results of the randomized clinical study in 36 TKA surgery patients, who underwent two 3-step perioperative mVLT sessions each (total 72 mVLT sessions), for the investigation of method's capability to discriminate between pre-operatively dehydrated (12 hr fasting) and postoperatively more hydrated (24 hrs in ICU) subjects. The moderate rather than minimal test boluses were applied—5 ml kg⁻¹ of Ringer's acetate—which is twice the currently recommended test bolus. Aside from the missing possibility to compare the two positive arterial VPR values in order to apply the diagnostic criteria of the mVLT, the diagnosis of the postoperatively better hydration status can still be carried out. It is based on the finding that postoperative VPR was positive in the 3^{rd} step after being negative in the 2^{nd} step. It means that in step two it was the dilution plateau which is the last interval of the hydration plateau (see FIGS. 38 and 42). The preoperative VPR is negative in both 2^{nd} and 3^{rd} steps suggesting the states within the two phases of the hydration dilution plateau, e.g. the 2^{nd} step was in the lymphatic plateau, and 3^{rd} was in the dilution plateau (see FIGS. 38 and 42). Also, the amplitude of the postoperative VPR shift was significantly lower than preoperative.

FIG. 53 depicts the results of the randomized clinical study in 36 TKA surgery patients, who underwent two 3-step perioperative mVLT sessions each (total 72 mVLT sessions), for the investigation of method's capability to discriminate between pre-operatively dehydrated (12 hr fasting) and postoperatively more hydrated (24 hrs in ICU) subjects. The moderate rather than minimal test boluses were applied—5 ml kg⁻¹ of Ringer's acetate—which is twice the currently recommended test bolus. Aside from the missing possibility to compare the two positive capillary VPR values in order to apply the diagnostic criteria of the mVLT, the diagnosis of the postoperatively better hydration status can still be carried out. It is based on the finding that the amplitude of the postoperative VPR shift from bigger, what means that postoperative shift has started earlier inside the hydration plateau, e.g. the first postoperative hydration shift started in the lymphatic plateau, while postoperative started in the dilution plateau (see FIGS. 38 and 42). Moreover, according to the mVLT method, the capillary trends (single-capillary bed model) go slightly in front of arterial trends (multiple capillary beds model). All that supports the need to apply more steps with lower volume test boluses rather than lower number of steps with bigger volume of test boluses. The relatively long averaging time (60 sec) applied by the apparatus for the noninvasive measurement of capillary Hb (SpHb™) may add to the cutting of peaks in the parameter deviations.

FIG. 54 depicts the results of the randomized clinical study in 36 TKA surgery patients, who underwent two 3-step perioperative mVLT sessions each (total 72 mVLT sessions), for the investigation of method's capability to discriminate between pre-operatively dehydrated (12 hr fasting) and postoperatively more hydrated (24 hrs in ICU) subjects. The moderate rather than minimal test boluses were applied—5 ml kg⁻¹ of Ringer's acetate—which is twice the currently recommended test bolus. Aside from the missing possibility to compare the two positive venous VPR values in order to apply the diagnostic criteria of the mVLT, the diagnosis of the postoperatively better hydration status can still be carried out. Just like in arterial trends, it is based on the finding that postoperative VPR was positive in the 3^{rd} step after being negative in the 2^{nd} step. It means that in step two it was the dilution plateau which is the last interval of the hydration plateau. The preoperative VPR is negative in both 2^{nd} and 3^{rd} steps suggesting the states within the two phases of the hydration dilution plateau, e.g. the 2^{nd} step was in the lymphatic plateau, and 3^{rd} was in the dilution plateau (see FIGS. 38 and 42). In contrast to arterial and capillary trends, the amplitude of the postoperative VPR shift was not significantly bigger.

FIG. 55 depicts a block diagram that illustrates a computer system 700 upon which an embodiment of the invention may be implemented

Table 1 depicts the assessment and monitoring of fluid balance.

Table 2 depicts the physiologic association of midcapillary hydraulic pressure and related arteriolar and venular tone in capillaries.

Table 3 depicts the generic parameter's—the hemoglobin concentration—relationship with its derivative variables derived by processing the generic value by equations of the BIRD-math. The derivative variables are described mathematically and physiologically, and their interpretation by the mVLT method as diagnostic criterial is shortly defined.

### ABBREVIATIONS AND DEFINITIONS

GDM—Goal directed (fluid) management
VLT—Volume loading test
SV—Cardiac stroke volume
PRBC—Packed red blood cells (collected for transfusion purposes)
avDD—Arterio-venous dilution difference
MCHC—Mean cell hemoglobin concentration
MCV—Mean cell volume
MCH—Mean cell hemoglobin content
iHP—interstitial hydrostatic pressure
iCOP—Transcapillary colloid osmotic pressure
cHP—Capillary hydraulic pressure
THP—Transcapillary hydraulic pressure
FAR—Transcapillary filtration-absorption ratio
AVTR—Arterio-venular tone ratio
THP—Transcapillary hydraulic pressure
TCOP—Transcapillary colloid osmotic pressure
mVLT—the minimal volume loading test.
Hydration or hydration status—the state or degree of the interstitial tissue hydration.
Bolus—a gravity or pump driven iv test load infusion targetted to infuse up to 5.0 ml kg⁻¹ in 5 minutes.
Test bolus—a bolus of relatively small or progressively increasing volume of isoosmotic crystalloid solution.
Crystalloids—the intravenous isoosmotic crystalloid solutions.
PD—plasma dilution.
Hb—blood hemoglobin concentration.
Hct—blood hematocrit.
Plasmadilution (PD)—the fractional change of hemoglobin concentration in respect to initial baseline.
Steady state—period without any kind of fluid, blood product or food administration to the person.
5′StS—the five minute steady state.
Minimal volume loading test (mVLT)—a series of minimal volume loading test steps.
Minimal volume loading test step—a relatively small or progressively increasing volume of iiv isoosmotic crystalloid solution followed by 5 min period without any fluid administration or intake.
mVLT step—the minimal volume loading test step.
Relatively small iv test volume load—is the 1.5-2.5 ml kg⁻¹ volume of isoosmotic crystalloid solution.
Progressively increasing iv test volume load—is the volume of isoosmotic crystalloid solution that exceeds the previous load by 25%, but the maximal recommended load is 5.0 ml kg⁻¹, although exceptions may apply in the states of massive hemorrhage or shock.
Checkpoint—the data point or time-point during the minimal volume loading test when (1) the blood sample(s) for the analysis of hemoglobin concentration(s) is obtained or noninvasive reading of hemoglobin concentration is recorded, and/or (2) other parameters are recorded, (3) and/or derivative variables are calculated from the measured parameters.
Peak—a checkpoint at the end of test bolus.
Acute residual—a checkpoint after the 5 minutes period without fluids that follows the test bolus.
Close residual—a checkpoint after the 20-30 minutes period without fluids that follows the test bolus.
Initial baseline—the checkpoint just before the start of the mVLT session.
Individual initial baseline applies only to parameters that have only one sampling site and/or method of measurement at the moment of evaluation.
Common initial baseline applies only to parameters that have more than one sampling site and/or method of measuring at the moment of evaluation (e.g., hemoglobin concentration) so that one site's reading serves as baseline for all measuring sites.
Individual baseline—the checkpoint just before the start of the iv infusion during the mVLT session.
Transitory or temporary hydration state is an unstable state of whole-body and plasma hydration that is after 5 min steady state without fluid following the test fluid challenge during mVLT
Whole-body or permanent hydration state is a relatively stable state of hydration that is after 20-30 min period without fluid following the last step mVLT.

### DETAILED DESCRIPTION

### The present invention provides the minimal Volume Loading Test (mVLT) method for the evaluation and modification of the whole body hydration

It Provides a Solution for All Deficiencies as follows:

### mVLT Method

### The Basics

Minimal Volume Loading Test (mVLT) implies evaluation of target parameters' response to the intravenous test fluid load (test bolus) of isoosmotic crystalloid solutions (crystalloids). Target parameters include but are not limited to those used in conventional goal directed fluid therapy such flow-related parameters (e.g. cardiac stroke volume), and the newly introduced by the present invention target parameter—the blood hemoglobin concentration (Hb), hematocrit (Hct) and their derivative variable—plasma dilution (PD).

The mVLT method deploys at least four consecutive processes or steps (mVLT steps). Each step consists of test bolus followed by the five minute steady state (5′StS) which is a period without any fluid, blood product or food administration to the individual. Steady state is used to allow the intercompartment distribution of the test bolus:
1. Acute residual fluid distribution period is referred to as 5 min steady state (5-StS),
2. Close residual fluid distribution period is referred to as 20-30 min steady state (20-30′StS), which is sometimes deployed after the last mVLT step.

### Test Bolus and Data Points

Target parameters derived at these data points (checkpoints) can be referred to as acute or close residual, respectively. However, in the following text, all variables are defined by the mVLT step number and refer to the acute residual checkpoints. Peak values obtained in checkpoints at the end of test bolus are not considered by the mVLT method since it is a very unstable state. The method implies the use of two major strategies of test bolus administration—the series of at least four (a) relatively small or (b) progressively increasing test boluses.

Relatively small test bolus is the 1.5-2.5 ml kg-1 volume load of crystalloid infused in a maximal available rate.

Progressively increasing test bolus is the crystalloid volume that exceeds the preceding test bolus, e.g. by 10-25%, but the maximal recommended test bolus is 5.0 ml kg⁻¹, although exceptions may apply in such cases as massive hemorrhage or shock. Also, the test bolus has to be context-sensitive, and first of all—clinically appropriate and safe depending on the preexisting status of an individual.

### Target Parameters in mVLT

The present inventor refers to all target parameters as generic parameters or generics since they are represented by the inherent measurable value of a specific parameter, e.g., generic parameter Hb is represented by its measured value expressed in g/L or g/dl.

### Application Algorithms in mVLT

In an embodiment, the present invention uses algorithms provided herein for the evaluation and optimization of fluid status by means of the mini Volume Loading Test (mVLT). The mVLT procedure has two objectives—diagnostics and optimization. Diagnostic mVLT (DmVLT) consists of at least four mVLT steps [FIG. 45], while optimizing mVLT (OmVLT) needs continuation of mVLT steps until the preset degree of transitory hydration is met [FIG. 46].

### Mathematical Model of Bolus Induced Response of Deviations

### BIRD-Math

Terms and equations of the BIRD-math are corresponding to the conventional terms and definitions in research and statistics (http://onlinestatbook.com/chapter3/variability.html; and http://en.wikipedia.org/wiki/Variation). In example, the calculation of the fractional change from baseline is deployed for the calculation of plasma dilution (PD) which is the Hb deviation from initial baseline (TAB.3). The shift of PD during a single mVLT step is referred to as plasma dilution difference (PDD), or plasma dilution efficacy, or response. The BIRD-math also deploys the calculation of variability which is ‘the state of being variable’, and usually describes how spread is the data in statistical analysis. There are four commonly used measures of variability: range, mean, variance and standard deviation. For the description of plasma dilution response tendency in two consecutive mVLT steps, the BIRD-math deploys arithmetic mean of plasma dilution (MPD) or just response variability. It is the sum of two consecutive PDD values divided by two.

The BIRD-math also deploys the calculation of variation which is ‘any perturbation of the mean motion’ commonly used in statistical analysis. Also, the variation of a flow-related parameter such as cardiac stroke volume variation (SVV) during a cycle of mechanical or even spontaneous ventilation of the lungs is conventionally referred to as dynamic parameter used for the prediction and monitoring of fluid responsiveness. The variation is conventionally calculated as difference of maximal and minimal values of the parameter divided by the mean value, where the minimal and maximal values are usually automatically detected over a period of time sufficient to include at least one complete respiratory cycle. In contrast to parameter's variation during a respiratory cycle where maximal value is associated with expiration and related increase in cardiac preload, the plasma dilution during mVLT changes in correspondence with multiple factors such as interstitial fluid compliance of and transcapillary filtration-absorption ratio (FAR)—local, regional and whole-body. Thus, during a single mVLT step, the plasma dilution can be minimal before the test bolus and maximal after the 5 min steady state, and vice versa. Consequently, the conventional calculation of variation and its interpretation is inappropriate for the mVLT method. Therefore, the BIRD-math deploys a modified calculation of the variation of plasma dilution response (VPR) or just response variation. It is simply the deviation of the last steps' plasma dilution difference (PDD) from the mean value of the last two steps (MPD). That variation which is, in a statistical language, ‘a perturbation of the mean motion’ has major importance for the detection of the hydration plateau. More specifically, the shift of the negative variation (VPR) to positive value takes part immediately after the center of the hydration plateau is reached. The higher the sum volume of interstitium that has gained the maximal fluid compliance in the center of the hydration plateau, the higher is the positive VPR value reached. Consequently, the increasing positive VPR during mVLT is a marker of the increasing volume of the capillary beds that gain maximal interstitial fluid compliance. Similarly, the decreasing positive VPR is a marker of the decreasing volume of the capillary beds that gain maximal interstitial fluid compliance. The overall excitability of plasma dilution is defined as absolute VPR value or just responsiveness (ABS-VPR). It reaches maximum during mVLT when, and if, the hydration plateau consistent with maximal sum interstitial fluid compliance is reached. It is associated with transitory optimized interstitial hydration status of the whole body.

### Equations of the BIRD-Math

Equations of the BIRD-math are defined for the evaluation of the plasma dilution responsiveness during the mVLT (TAB.3).

1. DEVIATION OF THE GENERIC PARAMETER. Continuous or total deviation of a parameter or variable in respect to initial baseline which is before the mVLt session:

1.1. Interferring PLASMA DILUTION [PD]. Interfering continuous plasmadilution (C_xDᵢ) or plasma dilution (xPDᵢ) calculation is applicable only for blood hemoglobin concentration (Hb) when several simultaneous measures are available, and at least one of them provides initial baseline blood hematocrit value (Hct₀). Calculation implies that the one common initial baseline values—Hb₀ and Hct₀—obtained from one specific site and/or provided by one specific blood analysis method serves as common initial baseline for deviation trends of all simultaneously measured Hb values during mVLT session:

xPDᵢ=(yHb₀·xHbᵢ⁻¹−1)·(1−yHct₀)⁻¹ [1]

where xPDᵢ (or C_xDᵢ) is the Plasma Dilution at Checkpoint i at a Specific Measuring Site x (or obtained by method x); and xHbᵢ is the Hb value obtained following 5 min after the test bolus in mVLT step i at a specific measuring site x (or obtained by method x); Hb₀ and Hct₀ are the common initial baseline values obtained from one (operator preferred) measuring site y, while the measuring site and method for obtaining the xHbᵢ value can be the same as for y or different (initial common baseline is the data point just before the first test bolus in the mVLT session). Note that the derived result is the dimentionless fractional change of Hb, so multiplying it by hundred provides the deviation in percentile (%).

### 1.2. Continuous PLASMA DILUTION [PDc]

Continuous plasma dilution (C_xDcᵢ or PDc) is calculation is applicable only to Hb when a single available measure or several simultaneous measures are available, and they do not provide the baseline blood hematocrit value (Hct₀). Calculation implies that individual initial baseline value of Hb₀ obtained from a specific site and/or provided by one specific blood analysis method serves as individual initial baseline for deviation trends of specific site's yHbᵢ value during mVLT session:

xPDcᵢ=(xHb₀·xHbᵢ⁻¹)·xHb₀⁻¹ [2]

where xPDcᵢ (or C_xDᵢ) is the plasma dilution at checkpoint i at a specific measuring site x (or obtained by method x); and xHb₀ is the Hb value obtained following 5 min after the test bolus in mVLT step i at a specific measuring site x (or obtained by method x); xHb₀ is the individual initial baseline value obtained from a specific measuring site x (individual initial baseline is the data point just before the first test bolus in the mVLT session). Note that the derived result is the dimentionless fractional change of Hb, so multiplying it by hundred provides the deviation in percentile (%).

### 1.3. Continuous DEVIATION [ZD]

Continuous deviation (C_ZDᵢ or ZDᵢ) is applied for any parameter or variable Z. Calculation implies that individual initial baseline value of Z₀ serves as individual initial baseline for deviation trends of a mVLT session:

ZDᵢ=(Z₀−Zᵢ⁻¹)·Z₀⁻¹ [3]

where ZDᵢ (or C_ZDᵢ) is the deviation of parameter Z at checkpoint i; and Zᵢ is the value obtained following 5 min after the test bolus in mVLT step i; Z₀ is the individual initial baseline value obtained (individual initial baseline is the data point just before the first test bolus in the mVLT session). Note that the derived result is the dimentionless fractional change of Hb, so multiplying it by hundred provides the deviation in percentile (%).

IN THE EQUATIONS BELOW, THE PLASMA DILUTION RELATED VARIABLES SERVE AS AN EXAMPLE HOW TO USE THESE CALCULATIONS FOR ANY ALTERNATIVE GENERIC PARAMETER (equation s 1-3) SUCH AS CARDIAC STROKE VOLUME, etc.

2. Response [PDD]

Available from the first mVLT step.

Response is the plasma dilution efficacy of a single mVLT step, also referred to as plasma dilution difference [PDD] or plasma dilution efficacy:

xPDDᵢ=xPDᵢ−xPDᵢ₋₁ [4]

where xPPDᵢ is the plasma dilution shift in mVLT step #i; xPDᵢ is the plasma dilution in mVLT step #i, and xPDᵢ₋₁ is the plasma dilution in the preceding mVLT step #(i-1), which is the initial baseline PD0 in the first mVLT step; thus, initial baseline PD₀ is always nil in continuous plasma dilution trends (1.2.) and in interfering plasma dilution trends (1.1.) of a parameter that is used as initial common baseline. x—the reference to the generic parameter (e.g. a—for arterial Hb).

3. Response Variability [MPD]

Available from the 2nd mVLT step.

Response variability is the mean plasma dilution efficacy of two consecutive mVLT steps (mean PDD), also referred to as mean plasma dilution difference [MPD]:

xMPDᵢ=0.5·(xPDDᵢ+xPDDᵢ₋₁) [5]

where xMPDᵢ is the mean plasma dilution efficacy of two consecutive mVLT steps #i and #i-1; and xPDDᵢ is the plasma dilution efficacy of mVLT step #i; and xPDDᵢ₋₁ is the plasma dilution efficacy of mVLT step #(i-1); x—the reference to the generic parameter (e.g. a—for arterial Hb).

### 4. Response Variation [VPR]

Available from the 2nd mVLT step.

Response variation is the last step's deviation of plasma dilution efficacy (PDD) from its tendency in the last two steps (MPD), also referred to as variation of plasma dilution response [VPR] or variation of plasma dilution efficacy:

xVPRᵢ=xPDDᵢ−xMPDᵢ [6]

where xVPRᵢ is the variation of plasma dilution response in mVLT step #i; and xPDDᵢ is the plasma dilution efficacy of mVLT step #i, and xMPDᵢ is the mean plasma dilution efficacy of two consecutive mVLT steps #i and #i-1; x—reference to the generic parameter (e.g. a—for arterial Hb).

### 5. Responsiveness [ABS-VPR]

Available from the 2nd mVLT step.

ABS-xVPRᵢ=absolute value of xVPRᵢ.

Responsiveness is the overall excitability of plasma dilution represented by absolute value of the deviation of plasma dilution efficacy from its tendency in two consecutive steps.

The overall evaluation of plasma dilution responsiveness is not limited to the above equations since numerous modifications can also be applied, e.g. responsiveness can be evaluated by calculating the difference of response variation in two mVLT steps, but that variable would be available only from mVLT step #3. Nevertheless, it can be useful since it serves as an ‘amplifier’ of ABS-VPR.

### Physiologic Model of Bolus Induced Response of Deviations

### BIRD-phys

The predecessor of mVLT—the volume loading test (VLT) method—deploys two relatively large test boluses (7.5-10.0 ml/kg), and assumes that the bolus which is followed by the more pronounced plasma dilution after the 20′ StS shows that individual was less hydrated at baseline than he was before the bolus followed by the less plasma dilution after the 20-30′StS [FIG. 39]. As a simplistic physiologic background, the increasing interstitial fluid compliance was considered the main factor in the decrease of intravascular fluid retention. In addressing the concerns regarding possible fluid overload, the VLT was modified into the 3-step moderate volume loading test (3-step mVLT) where smaller test boluses (5.0 ml/kg) are infused over 5 min followed by the 5′ StS [FIG. 40]. The physiologic background of the latter development is that individuals who have a fluid deficit would presumably require more test boluses than those who are better hydrated in order to reach the same degree of interstitial hydration and fluid compliance [FIG. 41]. However, aside from the remaining risk of fluid overload, the moderate volume infusion can lead to missing the important markers of the hydration shifts. Thus, inventor proposed the present invention—the minimal volume loading test (mVLT). It deploys mVLT steps which are the relatively small test boluses (1.5-2.5 ml kg⁻¹) infused at maximal available rate and followed by 5 min steady states. For the most reliable diagnosis of the preexisting degree of interstitial tissue hydration, at least four mVLT steps are required. Meanwhile, aiming for the verification and especially for the modification of the hydration status more steps can be administered. The theoretical plasma dilution course during the idealized (theoretical) 6-step mVLT [FIGS. 42,43] is based on the new physiological model—the physiologic model of bolus induced response of deviations. The BIRD-phys model acknowledges that (a) immediate fluid responsiveness of plasma dilution is mostly dependent on the transcapillary flux guided by Starling forces since urine output and other routes of fluid elimination are much slower, and (b) transcapillary colloid-osmotic and hydrostatic pressures are the main determinants of the related transcapillary fluid shift (drag). The linear relationship of interstitial volume to pressure ratio (compliance), also the interstitial pressure to lymphatic flow ratio are well known (20,59) in both rehydration and overhydration of the interstitial tissues. However, their association with plasma dilution responsiveness has never been considered. The physiologic model of bolus induced response of deviations (BIRD-phys) is proposed by the inventor for the explanation of the physiologic processes behind the mVLT. Model defines the interstitial hydration degree related patterns of derivative variables that are obtained by processing the generic parameter—hemoglobin concentration (Hb) which is measured during the idealized (theoretical) 6-step-mVLT—by equations of the BIRD-math [FIGS. 37,38,42,43].

The states of interstitial hydration achieved in each mVLT step are transitory (relatively unstable after the 5′ StS that follows the test bolus), but they can be turned into permanent (relatively stable) when the last mVLT step is followed by about 20 min long steady state (20′StS). Acknowledging the instability of interstitial hydration states reached in the course of mVLT, the inventor refers to them as temporary or transitory hydration states. The arbitrary transitory interstitial hydration degrees [FIGS. 37,38] that correspond to data points at the end of 5′StS in every step of the idealized (theoretical) 6-step mVLT [FIGS. 42,43] are defined as follows: (a) data point 0 (before the test bolus of mVLT step #1)—severe dehydration, (b) data point 1 (end of mVLT step #1 which is after 5′ StS following test bolus #1)—moderate dehydration, (c) data point 2 (end of mVLT step #2 which is after 5′ StS following test bolus #2)—mild dehydration, (d) data point 3 (end of mVLT step #3 which is after 5′ StS following test bolus #3)—optimal hydration, (e) data point 4 (end of mVLT step #4 which is after 5′ StS following test bolus #4)—maximal normohydration, (f) data point 5 (end of mVLT step #5 which is after 5′StS following test bolus #5)—moderate overhydration, (g) data point 6 (end of mVLT step #6 which is after 5′ StS following test bolus #6)—severe overhydration. Shifts between these transitory hydration degrees (states) are referred to as following: (1) A—initial rehydration, (2) B—complete rehydration, (3) C—optimization, (4) D—maximization, (5) E—overhydration, and (6) F—overloading hydration.

### Hydration Plateau

Degrees of interstitial hydration are defined by the present invention in association with the following corresponding variables as follows: (a) interstitial hydrostatic pressure (iHP), (b) interstitial fluid volume (iFV), (c) interstitial lymph volume (L-vol), (d) lymphatic flow (L-flow) into circulation, (e) transcapillary metabolic oncotic drag (MOD), (f) compliance related anatomic hydraulic drag (AHD), and (g) total plasma dilution (PD) observed during the mVLT [FIG. 37]. The physiologic background of the mVLT deployed evaluation of plasma dilution responsiveness lies in the relationship of these variables in accordance with the hydration degree of the interstitium. The interstitial volume-pressure relationship is found linear in derma and muscles during rehydration and initial overhydration, but interstitial hydraulic pressure becomes constant despite increasing volume of interstitial fluid along a kind of plateau that lies between these states [FIG. 28] (20,59). The present inventor refers to it as a hydration plateau which is a degree of interstitial hydration [see hydration degrees #2 to #4 in FIGS. 37,38] associated with infinite interstitial volume-pressure ratio in derma and muscles.

The mVLT in a Single Dermal Capillary-Lymphatic Bed Model

A single capillary-lymphatic bed in derma (capillary bed later in the text) is a theoretical model that investigates the semi-isolated capillary bed assuming that no other tissues affect the plasma dilution of arterial blood that enters the capillaries, but implies that lymphatic flow originating from that capillary bed affects arterial plasma dilution. The inventor discovered that hydration plateau of a single capillary-lymphatic bed model is detectable in the course of mVLT by the minimization of plasma dilution efficacy in a single mVLT step (PDD), after which the plasma dilution efficacy starts rising again.

The metabolic oncotic drag (MOD) is the metabolically induced transcapillary osmotic/oncotic pressure gradient that tends to increase the transcapillary fluid filtration-absorbtion ratio (FAR). It is the highest in the presence of severe interstitial dehydration (interstitial hydration degree 0) due to the dehydration origin hyperosmolality of interstitium. Its osmolality abruptly falls, and so falls the oncotic drag during the transitory initial rehydration of interstitium (initial rehydration shift A between interstitial hydration degrees 0 and 1) which takes part in the 1^{st} step of the idealized 6-step mVLT. Thus, despite the increase of anatomic hydraulic drag (AHD) that tends to increase the filtration-absorbtion ratio during the expansion of interstitium by fluids, the rise of total plasma dilution is only minimal-to-moderate during the initial rehydration (see plasmadilution trend PD or residual continuous Hb dilution in FIG. 38). That is because of the stable sum filtration-absorbtion ratio which is a result of negative counteraction between the decreasing metabolic oncotic drag and increasing anatomic hydraulic drag. In a capillary level, it allows the ‘metabolic cleanup’ of interstitium via vasomotion which is the interchanging prevalence of transcapillary filtration and absorption induced by regular fluctuations in arteriolar and venular tone. In parallel, the interstitial hydrostatic pressure (iHP), the lymph volume (L-vol) and lymphatic flow (L-flow) into circulation are slowly increasing. The increase of the lymphatic flow into circulation contributes to the plasma test bolus induced plasma dilution of arterial blood that enters capillaries. That fraction can also ameliorate the capillary hemoconcentration affect arising from the possible increase of filtration-absorption ratio in case if the decrease of metabolic oncotic drag is lower than increase of anatomic hydraulic drag. Mid-capillary plasma dilution is equal to arterial since sum filtration-absorption ratio remains unchanged, as described above. The two derivative variables of the BIRD-math available in mVLT step 1 [TAB.3] are the total plasma dilution of a single step (PD) and plasma dilution difference (PDD) of a single step. It is positive in both arterial and capillary blood during the transitory initial rehydration of interstitium.

During the transitory completion of interstitial rehydration (complete rehydration shift B between interstitial hydration degree 1 and 2) is associated with the 2^{nd} step of idealized 6-step mVLT. The iHP, L-vol and L-flow steeply increase until reaching the 2^{nd} degree of transitory interstitial hydration. In parallel, the following deviations reach the maximum—the decrease of metabolic oncotic drag and the increase of anatomic hydraulic drag. Thus, they neutralise each others affect on the filtration absorption ratio which remains constant. Consequently, the total plasma dilution (PD in FIG. 38) is steeply increasing, and that increase is significantly facilitated by the increasing L-flow. The five derivative variables of the BIRD-math available in mVLT step 2 are the total plasma dilution of a single step (PD), plasma dilution difference (PDD) of a single step, mean PDD of the two steps (MPD), the deviation of PDD from the mean value or MPD also referred to as response variation (VPR), and the absolute value of the latter variable also referred to as responsiveness (ABS-VPR). The non-specific signature of the transitory complete interstitial rehydration are the positive values of all these variables [TAB.3].

The hydration plateau is also referred to as hydraulic plateau aiming to emphasize that interstitial pressure does not change despite increasing interstitial fluid volume. Meanwhile, there are several more plateau patterns within the hydration plateau.

During the transitory optimization of interstitial hydration (optimization shift #C) which is the 3^{rd} step of the idealized 6-step mVLT, the lymphatic status quo (lymphatic plateau) is reached—the iHP, L-vol and L-flow all become constant. In the beginning of the hydration plateau [see hydration degree #2 in FIGS. 37,38], the interstitial tissue fluid compliance reaches maximum. Nevertheless, the plasma dilution slowly increases further in the shift of hydration degree from 2 to 3 which is along the lymphatic plateau where L-vol and L-flow do not change [see hydration degree #3 in FIGS. 37,38]. Plasma dilution is promoted by the initially slow decrease of interstitial fluid compliance. Same all five derivative variables of the BIRD-math are available in mVLT step 3. The specific signature of the transitory optimized interstitial hydration are the negative values of PDD and VPR [TAB.3]. In the further shift of hydration degree from 3 to 4 which is along the dilution plateau the plasma dilution remains unchanged, while the L-vol decreases but L-flow increases [see hydration degree #4 in FIGS. 37,38]. Thus, despite the increasing interstitial fluid compliance, the evacuation of the lymphatics frees some space for interstitial fluid and does not allow the rise of hydraulic pressure. Also, the metabolic flush of ‘waste’ into interstitium from rehydrating cells raises proper transcapillary oncotic drag of fluid, all that resulting in the unchanging plasma dilution. The hydration degree 4 is the end of the hydration plateau. Same all five derivative variables of BIRD-math are available in mVLT step #4. The specific signature of transitory maximized interstitial hydration are negative values of PDD and VPR in association with the close to nill or negative PD [TAB.3].

In the further shift of hydration degree from 4 to 5 which is the ‘plateau exit’, plasma dilution steeply raises mainly as a result of decreasing both anatomic hydraulic and metabolic oncotic drag also referred to as decreasing overall interstitial fluid compliance. The L-vol further decreases and L-flow also starts decreasing [see hydration degree #5 in FIGS. 37,38]. The same all five derivative variables of the BIRD-math are available in mVLT step #5. The specific signature of the transitory interstitial overhydration are the positive values of PDD and VPR that follow their negative values (in the preceding step) in association with the positive PD [TAB.3].

In the further shift of hydration degree from 5 to 6, the plasma dilution further raises mainly as a result of further decreasing overall interstitial fluid compliance. The L-vol and L-flow are further decreasing and approach basal values [see hydration degree #6 in FIGS. 37,38]. The same all five derivative variables of the BIRD-math are available in mVLT step #6. The non-specific signature of the transitory interstitial overhydration are the positive values of PDD and VPR that follow their positive values (in the preceding step) in association with the positive PD [TAB.3].

The mVLT in Multiple Capillary Bed (Whole-Body) Model

### Regional and Whole-Body Hydration

The plasma dilution in a single dermal capillary bed is dependent on the plasma dilution of arterial blood that enters that capillary bed. As arterial plasma dilution is a reflection of the whole body transcapillary distribution of infused test bolus, its course of plasma dilution is dependent on the hydration, and consequently—the sum interstitial fluid compliance, of all the perfused capillary beds. Meanwhile, the hydration of the latter is not uniform within the body, and perfusion regions are expanding and contracting depending on the constellation of endogenous and exogenous factors.

### Concept of Multiple Hydration Plateaus

The inventor discovered that on the way to the maximal sum interstitial tissue fluid compliance of the whole-body, the series of hydration plateaus can be seen in arterial blood during the mVLT. In dehydration, the capillary plasma dilution, if monitored in a single capillary bed, would first approach the hydration plateau of its own interstitium adjacent to its capillaries, and its detection would be minimally affected by the arterial plasma dilution. Initial rehydration affects the central organs and periphery such as derma. The rehydration of the central compartments takes part first, but these compartments do not have big anatomic interstitial volume and fluid compliance, so the impact of changes in their compliance on systemic plasma dilution is initially negligible. The simultaneously involved capillary beds in derma of the whole body act in a very similar way as a single capillary bed, and lead to a similar pattern of arterial and capillary plasma dilution. The opening of previously restricted capillary beds other than in derma tend to increase the arterial plasma dilution, and that increase is progressive in correspondence with the increasing volume of opening capillary beds. Further in the hydration advance, the influence of arterial plasma dilution on capillary plasma dilution grows, and finally capillary plasma dilution becomes a reflection of arterial plasma dilution since no big changes in capillary filtration-absorption ration are available after the single-capillary-bed interstitium reaches the low compliance due to maximized fluid content. Consequently, the monitoring of plasma dilution in a single-capillary-bed can provide information about both—the local interstitial degree of hydration and its changes during mVLT, and the sum interstitial hydration of the whole-body. Thus, the mVLT method is suitable for both—the evaluation of transitory hydration degree(s) in a single single-capillary-bed and the whole-body.

### Major Markers of the Changing Degree in the Whole-Body Interstitial Hydration

As described in the previous text, there will be a series of hydration plateaus in both arterial and capillary plasma dilution trends during the shift of transitory hydration degree of regional/local and the whole-body interstitium. Due to the limitations and deficiencies arising mostly from the lack of specificity [TAB.3], only the response variation (VPR) is the most important for mVLT. The responsiveness which is an absolute VPR value is only a supportive variable. The present inventor discovered that plateaus on the way to the sum maximized interstitial fluid compliance of the whole body capillary beds will be detected by the increasing positive values of response variation (VPR) in both arterial and a single capillary-bed trends that are derived simultaneously. The positive VPR starts decreasing after the exit from the very specific hydration plateau which is associated with the maximal sum interstitial fluid compliance or transitory maximized interstitial hydration of the whole body or just maximized whole-body hydration. That particular degree of interstitial hydration in the capillary-bed-dependend interstitium is reached something earlier than that of the whole body. At that point, the capillary plasma dilution becomes progressively more dependent on the arterial plasma dilution, and thus starts providing the monitoring of the whole body hydration shifts without tracing the arterial Hb. Venous plasma dilution is affected by interfering and continuously changing factors such as rate of capillary perfusion and arteriovenous shunting, also the filtration absorbtion ratio in the ‘feeding’ capillary beds. Thus, venous plasma dilution may need the specific approach which still has to be discovered. At the current state of the mVLT method development, the venous plasma dilution trends have to be interpreted with caution.

### Relationship of Capillary Plasma Dilution and Total Hemoglobin (SpHb™)

Derma is conventionally used for the observation of microcirculation [FIGS. 21-23], and more recently—for non-invasive real-time continuous monitoring of capillary hemoglobin concentration [60], e.g. monitoring total hemoglobin (SpHb™) with a device connected to the probe placed on the nail of the finger and/or any other applicable site of derma (Masimo Rainbow SET® Radical 7; Masimo Corporation, USA). Aiming to reach the more stable readings, the device uses the averaging of data which 60 sec at minimum. It is said that SpHb™ is the Hb measure in a pulsatile part of interstitium, and consequently it is supposed to be the metarteriolae rather than capillary. Furthermore, SpHb™ is addressed as arterial or venous depending on the mode of operation of the device—the “arterial” or “venous” mode can be chosen on a device by the operator. The venous SpHb™ is however derived solely mathematically from the presumed arterial value. The currently appearing in literature reports (60) do not find the sufficiently good correlation of SpHb and conventional invasive measures of Hb. And that is what could be expected from the physiologic point of view. More specifically, the pulsatile fraction of dermal interstitium includes not only the metarteriolae where pure arterial blood passes directly in to venulae, but also the midcapillary section of capillaries as it is also pulsatile even as a result of neuro-humorally guided vasomotion. The changing arteriolar and venular tone in response to the test boluses of the mVLT adds to the pulsatility of the capillaries. Consequently, the SpHb™ is a surrogate of metarteriolar (arterial) Hb and midcapillary Hb of a single capillary bed rather than arterial or venous Hb. The described theoretical arguments also explain why the arterio-capillary and veno-capillary dilution differences are not equal and they are changing during the mVLT. Nevertheless, if that theory is true, the deviations of SpHb™ derived plasma dilution from arterial dilution during mVLT are solely dependent on the corresponding deviations of arterio-capillary dilution difference which, in turn, is solely dependent on the changes of transcapillary filtration-absorption ratio. Consequently, the deviation of plasma dilution derived from SpHb™ is a reflection of simultaneous deviation of capillary plasma dilution in the dependent capillary bed. Thus, deviations of SpHb™ derived plasma dilution can be deployed for the purposes of capillary plasma dilution evaluation by the mVLT method, and enable the totally noninvasive continuous real time applicability of the mVLT method. Shortening of the SpHb™ averaging time to the minimal technically available duration would be beneficial aiming to the better detection of the peaks and falls of capillary plasma dilution since they are of major importance in the evaluation plasma dilution response variations and the overall fluid responsiveness.

### Relationship of Capillary Plasma Dilution and Other Capillary Flow Related Parameters

Monitoring SpHb™ with a device connected to the probe placed on the nail of the finger and/or any other applicable site of derma (Masimo Rainbow SET® Radical 7; Masimo Corporation, USA) also provides the parallel readings of perfusion index (PI).

The PI is calculated by indexing the infrared pulsating signal (presumably derived from pulsating arterial blood) against nonpulsatile signal (presumably derived from skin, other tissues and nonpulsatile blood), and expressed as percentage [61]. Furthermore, the pleth variability index is mathematically derived and displayed by the same apparatus. It is the reflection of PI changes during a period of time sufficient to include at least one complete respiratory cycle. Thus, in origin, the PVI is the mathematically processed PI. Meanwhile, the latter, as described above, is dependent on the ratio of pulsatile and nonpulsatile interstitium, and pulsatile and nonpulsatile blood.

Just like with SpHb™, the PI is highly dependent on the surrogate of the pulsatile fraction of dermal interstitium that includes the metarteriolae and the midcapillary section of capillaries since it is also pulsatile even at rest due to vasomotion. The changing arteriolar and venular tone in response to the test boluses of the mVLT changes their pulsatility. Moreover, during crystalloid fluid administration, the accumulation of interstitial fluid would tend to decrease the PI, but can be opposed by the increasing blood flow in the pulsatile segments—metarteriolae and midcapillary section. Consequently, the prevailing tendency determines the changes in PI (and PVI) during mVLT and other strategies of fluid administration. The changing intrathoracic pressure such as seen during mechanical lung ventilation will interact with the above mentioned factors—can be prevailing or blunted by the former. That is why, theoretically, the PI and PVI are context-sensitive, and cannot reliably determine the fluid responsiveness and volume.

In the observations of the inventor, there is no reliable correlation between PI and the reading and even the readability of the SpHb™ parameter by the apparatus. The manufacturer's guidelines suggest that un-readability of the SpHb™ parameter is expected with low PI, but in reality the readings are available with the extremely low PI, and become un-readable with relatively high PI in the same subject on the same occasion. Consequently, theoretically, both parameters—the PI and PVI—cannot be reliably deployed by the mVLT method and should be addressed with caution when administrating intravenous fluids according to the conventional algorithms for the goal directed fluid therapy. Finally, the elaboration of the mVLT method can provide the new approach to the evaluation of PI and PVI dynamics, also providing clinically useful information.

### Relationship of Capillary Plasma Dilution and Cardiac Stroke Volume

The present inventor discovered that, theoretically, a strong correlation between the fluid responsiveness of capillary plasma dilution and cardiac stroke volume (SV) exists during the mVLT applications since both variables are plasma dilution dependent and are similarly affected by the same neuro-humoral factors such as sympathetic stimulation—it increases myocardial contractility and capillary plasma dilution. The latter is a result of increasing arteriolar tone that leads to the decrease of midcapillary hydrostatic pressure which, in turn, leads to the decrease in transcapillary filtration-absorption ratio that decreases capillary Hb.

### Diuretic mVLT Algorithm

Diuretics can be injected intravenously instead of test fluid bolus if the release of the edema-related fluid is the target. Specific deviations of plasma dilution will be seen if evaluated by the mVLT and the BIRD-math. Initially, the plasma dilution will be induced by the diuretic induced flux of fluid from interstitium into blood vessels. Then the plasma dilution will be seen returning to baseline when fluid is being excreted by kidneys. Diuretic injections should be continued until the diuretic-induced plasmadilution becomes negligible and/or plasma dilution decreases below baseline which was before start of the diuretic mVLT algorithm.

### Diagnostic Criteria in the mVLT Method

Based on the above, the diagnostic criteria for the monitoring of changes in the degree of hydration are proposed by the inventor:
1. Increasing positive values of a single capillary-bed derived response variation (VPR) and supported by increasing responsiveness (ABS-SRV) are markers of the on-going transitory optimization of interstitial hydration in that single capillary-bed-dependent interstitial tissues and the whole body.
2. Slowly decreasing positive values of a single capillary-bed derived response variation (VPR) and supported by decreasing responsiveness (ABS-SRV) are markers of the trend towards transitory maximisation of interstitial hydration in the single capillary bed-dependent interstitial tissues but maximisation may not be reached in the interstitium of the whole body (since capillary-bed-dependent interstitium reaches the maximal interstitial fluid compliance earlier that interstitium of the whole body; thus, maximisation of the VPR value in arterial trends may not be complete at that point).
3. Steeply decreasing positive values of a single capillary-bed-derived response variation (VPR) and supported by decreasing responsiveness (ABS-SRV) is a marker of the shift towards transitory maximization of interstitial hydration in the whole body.
4. Aiming to transform the transitory optimised state of hydration into relatively permanent, there are two options: (a) after reaching the criteria of transitory maximization, the maintenance infusion of crystalloids is set up to maintain the recorded at that point capillary Hb value, or (b) the mVLT steps should be continued until the minimization of the decreasing positive VPR supported by the decreasing responsiveness (ABS-SRV), and then make a 20 pause in fluid administration, trace the changes in capillary Hb and, when it returns to the value previously recorded in the step consistent with criteria of transitory maximization, initiate the maintenance infusion of crystalloids which is set up to maintain the recorded at that point capillary Hb value, or (c) the maintenance fluid infusion has to be tailored to maintain the plasma dilution reached at a specific mVLT step.
5. Baseline state's hydration status is determined when the first criteria of the transitory hydration state are met: (a) the increasing positive VPR and related diagnosis of transitory rehydration going on determines the diagnosis of baseline dehydration; (b) the decreasing positive VPR and related diagnosis of transitory overhydration going on determines the diagnosis of baseline normohydration; (c) the minimized positive VPR and related diagnosis of severe transitory overhydration going on determines the diagnosis of baseline moderate to severe overhydration.
6. Two consecutive negative VPR defines the hydration status within the hydration plateau, and normally will be followed by positive VPR, but the persistently negative VPR is associated with severe blood loss (persistent extravasation by entering the lymphatic loop for the endogenous recovery of plasma volume via fymph influx) or transcapillary leak, e.g. sepsis. When hemorrhage related demands for interstitial fluid are satisfied or septic extravasation of fluid resides, the VPR becomes positive.
7. Indirect evaluation of cardiac stroke volume's responsiveness: (a) increase in stroke volume is associated with arteriolar tone decrease and/or venular tone increase, and thus it will be seen as positive capillary VPR value; (b) decrease in stroke volume will be associated with arteriolar tone increase and/or venular tone decrease, and thus it will be seen as negative capillary VPR value.
8. Supportive interpretation of perfusion index (PI) and pleth variability index (PVI): (a) the PI increase associated with positive VPR means that capillary flow increases, arteriolar tone increases or constant, venular tone decreases or constant, stroke volume decreases or constant, arterio-venous shunting ratio-constant, transcapillary filtration-absorption ratio decreases, and finally—the PVI is high and/or increasing; (b) the PI decrease associated with positive VPR means that capillary flow increases, arteriolar tone increases or constant, venular tone decreases or constant, stroke volume decreases or constant, arterio-venous shunting ratio decreases, transcapillary filtration-absorption ratio decreases, and finally—the PVI is constant (low or high depending on the amplitude of fluctuations in PI and capillary flow); and (c) the PI decrease associated with negative VPR means that capillary flow decreases, arteriolar tone decreases or constant, venular tone increases or constant, stroke volume increases or constant, arterio-venous shunting ratio—constant, transcapillary filtration-absorption ratio increases, and finally—the PVI is decreasing. Other combinations apply according to the same principles that are based on the assumption that increasing capillary flow via decreased arteriolar tone and/or increased venular tone leads to significant increase of PI and decrease of capillary Hb marked by positive VPR.
9. The severe to moderate dehydration specific patterns are also the markers of hemorrhage, especially if preceded by the optimizing mVLT.
10. Increasing arterio-capillary dilution difference is the sign of increasing arterio-venous shunting.

It should be emphasized that the major diagnostic criteria described above (e.g. described within the “Diagnostic criteria in the mVLT method” section) are applicable to all of arterial, capillary and venous trends.

### Multimodal Feedback Loop (MFL) Algorithm and Computing Device

The present invention provides a Multimodal Feedback Loop (MFL) algorithm and the principle of the MFL device for the optimization of fluid and transfusion management.

It Provides a Solution for all Deficiencies, as follows:

The present invention provides a Multimodal Feedback Loop (MFL) algorithm based on the simultaneous evaluation of simultaneous response of multiple target parameters to the fluid load by simultaneous monitoring of trends described by the BIRD model. The MFL device uses software that deploys mathematical models of MFL algorithm and mVLT. The device can have one main and several optional inputs, also one main and several optional outputs. Main input can have, independently, one or two modules—e.g. manual and automated—for entering the hemoglobin concentration data, which is recorded at specific checkpoints during the processes used in mVLT. Hemoglobin data input has three arms since hemoglobin concentration can be obtained from arterial and/or venous and/or microvascular blood depending on available and applicable invasive and/or noninvasive methods. Automated hemoglobin data input can be provided via interface with devices that provide an electronic value of the parameter. Otherwise, hemoglobin data can be entered via manual input portal. Optional inputs are divided into feedback and data input terminals. The feedback input is for the feedback interface with fluid and transfusion pumps. The data input—manual and automated—includes but is not limited to flow-related parameters such as cardiac stroke volume, end diastolic filling ratio (EDFR), global end diastolic volume (GEDV) and systemic vascular resistance (SVR). Main output is for digital outflow of all data that is entering input terminals, and also the data derived from mathematical processing of input data according to the MFL algorithm and mVLT. More specifically, the later output provides definition of the suggested preexisting fluid status before and after mVLT, also suggesting further measures targeted to reach and/or maintain the specific fluid status. Optional output is divided into three operational interfaces with the three branches of infusion systems: crystalloid, colloid and blood product infusing pumps. These branches are further divided into specific interfaces according to the type of fluid and blood product. The main purpose of the operational interface is the guidance of isoosmotic crystalloid infusion pumps so that they administer fluid boluses at the rate defined by mVLT protocol and in accordance with the predetermined steady states between boluses.

FIG. 55 is a block diagram that illustrates a computer system 700 upon which an embodiment of the invention may be implemented. Computer system 700 includes a bus 702 or other communication mechanism for communicating information, and a processor 704 coupled with bus 702 for processing information. Computer system 700 also includes a main memory 706, such as a random access memory (RAM) or other dynamic storage device, coupled to bus 702 for storing information and instructions to be executed by processor 704. Main memory 706 also may be used for storing temporary variables or other intermediate information during execution of instructions to be executed by processor 704. Computer system 700 further includes a read only memory (ROM) 708 or other static storage device coupled to bus 702 for storing static information and instructions for processor 704. A storage device 710, such as a magnetic disk or optical disk, is provided and coupled to bus 702 for storing information and instructions.

Computer system 700 may be coupled via bus 702 to a display 712, such as a cathode ray tube (CRT) or Liquid Crystal Display (LCD), for displaying information to a computer user. An input device 714, including alphanumeric and other keys, is coupled to bus 702 for communicating information and command selections to processor 704. Another type of user input device is cursor control 716, such as a mouse, a trackball, or cursor direction keys for communicating direction information and command selections to processor 704 and for controlling cursor movement on display 712. This input device can have two or more degrees of freedom in two axes, e.g. a first axis (e.g., x) and a second axis (e.g., y), that allows the device to specify positions in a plane.

The invention is related to the use of computer system 700 for determining blood states for patients and their doctors. According to one embodiment of the invention, parameters for determining blood states is provided by computer system 700 in response to processor 704 executing one or more sequences of one or more instructions contained in main memory 706. Such instructions may be read into main memory 706 from another computer-readable medium, such as storage device 710. Execution of the sequences of instructions contained in main memory 706 causes processor 704 to perform the process steps described herein. One or more processors in a multi-processing arrangement may also be employed to execute the sequences of instructions contained in main memory 706. In alternative embodiments, hard-wired circuitry may be used in place of or in combination with software instructions to implement the invention. Thus, embodiments of the invention are not limited to any specific combination of hardware circuitry and software.

The term “computer-readable medium” as used herein refers to any medium that participates in providing instructions to processor 704 for execution. Such a medium may take many forms, including but not limited to, non-volatile media, volatile media, and transmission media. Non-volatile media includes, for example, optical or magnetic disks, such as storage device 710. Volatile media includes dynamic memory, such as main memory 706. Transmission media includes coaxial cables, copper wire and fiber optics, including the wires that comprise bus 702. Transmission media can also take the form of acoustic or light waves, such as those generated during radio wave and infrared data communications.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a computer can read.

Various forms of computer readable media may be involved in carrying one or more sequences of one or more instructions to processor 704 for execution. For example, the instructions may initially be carried on a magnetic disk of a remote computer. The remote computer can load the instructions into its dynamic memory and send the instructions over a telephone line using a modem. A modem local to computer system 700 can receive the data on the telephone line and use an infrared transmitter to convert the data to an infrared signal. An infrared detector coupled to bus 702 can receive the data carried in the infrared signal and place the data on bus 702. Bus 702 carries the data to main memory 706, from which processor 704 retrieves and executes the instructions. The instructions received by main memory 706 may optionally be stored on storage device 710 either before or after execution by processor 704.

Computer system 700 also includes a communication interface 718 coupled to bus 702. Communication interface 718 provides a two-way data communication coupling to a network link 720 that is connected to a local network 722. For example, communication interface 718 may be an integrated services digital network (ISDN) card or a modem to provide a data communication connection to a corresponding type of telephone line. As another example, communication interface 718 may be a local area network (LAN) card to provide a data communication connection to a compatible LAN. Wireless links may also be implemented. In any such implementation, communication interface 718 sends and receives electrical, electromagnetic or optical signals that carry digital data streams representing various types of information.

Network link 720 typically provides data communication through one or more networks to other data devices. For example, network link 720 may provide a connection through local network 722 to a host computer 724 or to data equipment operated by an Internet Service Provider (ISP) 726. ISP 726 in turn provides data communication services through the world wide packet data communication network now commonly referred to as the “Internet” 728. Local network 722 and Internet 728 both use electrical, electromagnetic or optical signals that carry digital data streams. The signals through the various networks and the signals on network link 720 and through communication interface 718, which carry the digital data to and from computer system 700, are exemplary forms of carrier waves transporting the information.

Computer system 700 can send messages and receive data, including program code, through the network(s), network link 720 and communication interface 718. In the Internet example, a server 730 might transmit a requested code for an application program through Internet 728, ISP 726, local network 722 and communication interface 718. In accordance with the invention, one such downloaded application provides for the calculating of transfusion strategies as described herein.

The received code may be executed by processor 704 as it is received, and/or stored in storage device 710, or other non-volatile storage for later execution. In this manner, computer system 700 may obtain application code in the form of a carrier wave.

While this invention has been described in connection with the best mode presently contemplated by the inventor for carrying out his invention, the preferred embodiments described and shown are for purposes of illustration only, and are not to be construed as constituting any limitations of the invention. Modifications will be obvious to those skilled in the art, and all modifications that do not depart from the spirit of the invention are intended to be included within the scope of the appended claims. Those skilled in the art will appreciate that the conception upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

With respect to the above description then, it is to be realized that the optimum dimensional relationships for the parts of the invention, including variations in size, materials, shape, form, function and manner of operation, assembly and use, and all equivalent relationships to those illustrated in the drawings and described in the specification, that would be deemed readily apparent and obvious to one skilled in the art, are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

### CLINICAL CASE EXAMPLES

### Case 1 [FIGS. 47-50]

Randomized Crossover Study to Investigate the Capability of mVLT to Discriminate Between Hydrated and Dehydrated Healthy Young Volunteers

### Objective

The aim of the randomized crossover study was, by means of the mVLT method, to discriminate between hydrated and dehydrated healthy young volunteers.

### Methods

### Subjects

Each volunteer was subject to two 6-step mVLT sessions separated by at least two weeks. On both occasions they arrived after at least 12 hr of fasting.

### Monitoring and Interventions

An iv line for fluid infusion was set in the independent arm. In addition to standard perioperative monitoring, the other intravenous line for blood sampling was set in the other arm, and the sensor was placed on the middle finger for continuous noninvasive measurement of total hemoglobin (SpHb™ also referred to as capillary Hb-cHb) (Masimo Rainbow SET® Radical 7; Masimo Corp, USA). The averaging time for SpHb was set to “short” (1 min), and switched to “arterial” mode.

Immediately after the above procedures, on one occasion (referred to as DEHYDRATED experiment) the subjects stayed in a supine position for 45 min prior to mVLT. On another occasion (referred to as HYDRATED), subjects were given to drink 5 ml/kg of water and also stayed in a supine position for 45 min prior to mVLT.

6-step mVLT

Six test boluses of 2.5 ml/kg acetated Ringer's separated by the 5 min steady states were used during the 6-step mVLT session. Venous and capillary blood Hb was recorded before each test bolus (initial baseline or acute residual of the previous mVLT step), and after the 5 min steady state. The blood Hb was analyzed at bedside (HemoCue®, Sweden). Laboratory analysis of venous Hb and Hct was made only to determine the common initial baseline values that are needed for the calculation of plasma dilution—venous and capillary—in respect to that single (common) baseline.

Data Processing and Statistical Analysis Venous and capillary plasmadilution were processed by equations of the BIRD-math aiming to determine the preexisting whole-body hydration status. Mean values were compared by using Students t-test, and Levene's test was used for comparison of variances. Statistical analysis was performed by SPSS and P<0.05 was considered significant.

### Results

The total of ten 6-step mVLTs was performed on two occasions for five healthy volunteers. The mean venous plasma dilution was similar on both occasions (FIG. 48 A), but the capillary plasma dilution (derived from SpHb™ or cHb) was significantly more advanced in the mVLT for the better hydrated subjects (FIG. 48 B). Markers of hydration plateau—equal capillary PD (derived from noninvasive SpHb™) in two consecutive mVLT steps or PD that is lower than the PD of the preceding step—are shown in the rectangles of FIG. 48 B. Venous trends (FIG. 48 A) were not informative in that context. Note that the earlier appearance of capillary hydration plateau in less hydrated does not mean they were better hydrated according to the diagnostic criteria of the mVLT (TAB.3). All that matters is the trends of response variation and responsiveness as described in FIGS. 49 and 50.

### Venous Plasma Dilution Responsiveness

In steps 3 and 4 (FIG. 49), the less hydrated subjects presented with increasing positive mean venous plasma dilution response variation (VPR) value supported by the increasing responsiveness which is the absolute value of VPR. In contrast, the better hydrated volunteers presented with VPR and ABS-VPR variables decreasing from the beginning of their availablity—in a shift from step 3 to 5. According to the diagnostic criteria of mVLT method (also see TAB. 3), these findings are consistent with the presumed better baseline hydration in the latter group.

### Capillary Plasma Dilution Responsiveness

In steps 2 and 3 (FIG. 50), the less hydrated subjects presented with increasing positive mean response variation (VPR) value supported by the increasing responsiveness which is the absolute value of VPR. The trends began to decrease in a shift from step 3 to 5 and 6. In contrast, the better hydrated volunteers presented with VPR and ABS-VPR variables decreasing from the beginning of their availablity—in a shift from step 2 to 4, and further in a shift from step 4 to 6. According to the diagnostic criteria of the mVLT method (also see TAB. 3), these findings are consistent with the presumed better baseline hydration in the latter group.

### CONCLUSION

1. The 6-step mVLT provided discrimination between dehydrated and better hydrated healthy young volunteers.
2. The diagnostic conclusions of the mVLT were similar when invasive venous and noninvasive capillary Hb (SpHb™) measures were processed by the BIRD-math.

### Case 2 [FIGS. 51-54]

Prospective Clinical Trial to Investigate the Capability of mVLT to Detect the Difference of Baseline Hydration Between Preoperative and Postoperative Hydration in Patients

### Objective

The primary aim of the prospective clinical trial was, by means of the mVLT method, to determine the preexisting whole-body hydration status after the preoperative overnight fast in one occasion, and after the postoperative 24 hrs stay in ICU in another occasion, also to compare the preexisting hydration status between these two experiments. Secondary aim was to evaluate the correlation between simultaneously measured cardiac stroke volume and plasma dilution, arterial pressure and perfusion index.

### Methods

### Patients

Elective primary total knee arthroplasty (TKA) patients were subject to two mVLT sessions—preoperative and postoperative—separated by 24 hours including surgery and ICU stay.

### Monitoring

On the arrival to the operating theatre at 7 AM, an iv line for fluid infusion was set in the independent arm. In addition to standard perioperative monitoring, the cannulation of radial artery was performed for arterial blood sampling, also for the invasive continuous monitoring of ABP deviations (DASH 3000, GE Inc., USA), and deviations of non-calibrated cardiac stroke volume (SV) by means of arterial pulse contour analysis based technique (LiDCO™Plus, UK). Intravenous line for blood sampling was set in the same arm, and the sensor was placed on the middle finger for continuous noninvasive measurement of total hemoglobin (SpHb™ also referred to as capillary Hb-cHb) (Masimo Rainbow SET® Radical 7; Masimo Corp, USA). The averaging time for SpHb was set to “short” (1 min), and switched to “arterial” mode.

3-Step mVLT

Three test boluses of 5 ml/kg acetated Ringer's separated by the 5 min steady states were used during the 3-step mVLT session. Arterial, venous and capillary blood Hb was recorded before each test bolus (initial baseline or acute residual of the previous mVLT step), and after the 5 min steady state. The blood Hb was analyzed at bedside (HemoCue®, Sweden). Laboratory analysis of arterial Hb and Hct was made only to determine the common initial baseline values that are needed for the calculation of plasma dilution—arterial, venous and capillary—in respect to that single (common) baseline. Induction of spinal anaesthesia was performed immediately after the preoperative 3-step mVLT session.

### Surgical Interventions

Operations were performed by the same senior surgeon. Wound drainage closed during the trial.

### Data Processing and Statistical Analysis

Arterial, venous and capillary plasmadilution were processed by the equations of BIRD-math aiming to determine the preexisting whole-body hydration status. Mean values were compared by using Students t-test, and Levene's test was used for comparison of variances. Statistical analysis was performed by SPSS and P<0.05 was considered significant.

### Results

The total of 72 mVLTs was performed on two occasions for 36 TKA surgery patients. The mean preoperative arterial Hb (aHb) was significantly higher than postoperative (p<0.000), and difference of variances was not significant (p<0.061). Similarly, the mean preoperative venous Hb (vHb) was significantly higher than postoperative (p<0.000), and difference of variances was not significant (p<0.116). The mean preoperative capillary Hb (SpHb or cHb) was significantly higher than postoperative (p<0.003), and difference of variances was not significant (p<0.508). The difference between cHb and aHb was significant only in the 2^{nd} and the 3^{rd} postoperative mVLT steps (p<0.031, p<0.027, and p<0.014 accordingly).

Mean response variation (VPR) in all—arterial, venous and capillary plasma dilution—was significantly higher (p<0.05) in the postoperative mVLT suggesting that patients were better hydrated since they reached closer to the arbitrary transitory maximization of the whole-body interstitial hydration status than pre-operatively.

Good correlation was only between fluid responsiveness of SV and capillary plasma dilution (rxy=0.959, p=0.016), also between SV and MAP (rxy=0.893, p=0.035).

Conclusion
3. The mVLT provided discrimination between presumably dehydrated preoperatively and better hydrated postoperatively primary elective total knee arthroplasty patients.
4. The diagnostic conclusions of the mVLT were similar when invasive arterial, venous, and noninvasive capillary Hb measures were processed by the BIRD-math.
5. The deviations of noninvasive capillary Hb can be used for the indirect monitoring of simultaneous deviations of cardiac stroke volume if both parameters are processed by the BIRD-math.

### Discussion

In contrast to the earlier described volunteers' study, the twice bigger test bolus was infused, but there were only three mVLT steps instead of six. Nevertheless, the mVLT method detected the better hydration of patients after 24 hr stay in ICU which was presumed and clinically logical.

Conclusions
The proposed new method has demonstrated good performance in pilot studies on healthy volunteers and patients. The invention leads to improvement in patient safety and provides physiologically adequate basis for future studies investigating the processes related to optimization of intravenous infusion therapy, also blood component transfusion and blood saving strategies.
There are specific areas within the prior art that can be enhanced by the new method as follows: 1) individual evaluation and modification of hydration status; 2) noninvasive indirect evaluation of cardiac stroke volume response to fluid challenges; 3) infusion and transfusion therapy measures; 4) monitoring of the diuretic therapy, and 5) trends for the future research.
The potential solutions and suggestions for each of these areas are specified as following: 1. The evaluation and optimization of circulation currently needs sophisticated and mostly invasive monitoring of flow related parameters such as stroke volume. Nevertheless, there is still a threat that optimization of cardio-vascular performance is reached in expense of deteriorated hydration status of an individual. The new method provides the non-invasive evaluation and modification of the hydration status and cardiac stroke volume. 2. Perioperative pulmonary edema related to infusion therapy remains an issue for decades and results in significant number of fatal outcomes. The mVLT deployed for planning and monitoring infusion therapy measures may prevent volume overload related threats. 3. The mVLT leads to the more accurate estimates of infusion and blood component transfusion amounts in approaching the clinical targets. 4. Unrecognized in a timely manner occult bleeding is a common cause of otherwise avoidable deaths. The mVLT for the detection of internal bleeding (see the diagnostic criteria for the detection of bleeding by the mVLT) can save lives by early indicating appropriate treatment. 5. The new method can lead to optimization of total intravenous anesthesia (TIVA) and improvement in patient safety by providing optimized plasma dilution. 6. The mathematical BIRD model is applicable for the evaluation of fluid responsiveness of different parameters. 7. In future experiments, the mVLT can be elaborated to fit the specific demands of the related fields of application. Development of methods for the clinical verification of fluid and volume status could be a priority.

### Alternative Embodiments

According to one or more alternative embodiments of this disclosure, there is provided:
1 A method for determining the state of hydration (diagnostic mVLT or DmVLT) of a subject comprising:
   a) quantifying the subject's initial baseline generic target parameters;
   b) intravenously administering to the subject an iso-oncotic, iso-osmotic non-cellular liquid at the highest safe rate over a period of 2-5 minutes, wherein the total volume administered does not exceed 5 ml per kg of subject body mass, and then quantifying the subject's peak generic target parameter(s) within 30 seconds of the end of the administering;
   c) quantifying the subject's acute residual generic target parameter(s) after a period of 5 minutes from the end of step b), but before 6 minutes from the end of step b) without further intravenous administration of a liquid to the subject;
   d) determining by formulae the derivative target parameter(s) for the respective generic target parameter(s) obtained in steps a), b) and c);
   e) intravenously administering to the subject an iso-oncotic, iso-osmotic non-cellular liquid at the highest safe rate over a period of 2-5 minutes, wherein the total volume administered does not exceed 5 ml per kg of subject body mass, and then quantifying the subject's peak generic target parameter(s) within 30 seconds of the end of the administering;
   f) quantifying the subject's acute residual generic target parameter(s) after a period of 5 minutes from the end of step b), but before 6 minutes from the end of step e), without further administration of liquid to the subject;
   g) determining by formulae the derivative target parameter(s) of the respective generic target parameter(s) obtained in steps e), f) and g);
   h) comparing the derivative target parameter(s) determined in step d) and step g) to a derivative-trend-matrix which is (i) FIG. 43B for derivatives defining arteriovenous dilution difference, (ii) FIG. 43C or FIG. 45 for derivatives defining arterial plasmadilution; (iii) FIG. 44B or 44C for capillary derivatives; and
   i) iteratively repeating steps e) through h) until the diagnosis of hydration status is derived from the derivative-trend-matrix or until the location on the ‘fluid status’ scale-axis in FIG. 43B, 43C or 45 is determined.
2. The method of clause 1 wherein the target parameter(s) are chosen from the group of perfusion index, tissue hemoglobin content, cardiac stroke volume, global end diastolic volume index, end diastolic filling ratio, and arterial, venous and capillary hemoglobin concentration ([Hb]), or wherein at least one of the target parameters(s) is chosen from this group, or wherein the group also comprises the other target parameter(s) as discussed in the specification.
3. The method of clause 1 wherein the target parameter is arterial [Hb] and/or capillary [Hb].
4. The method of clause 3, wherein the target parameters are arterial [Hb] and capillary [Hb].
5. The method of clause 3 or 4 wherein the capillary hemoglobin concentrations are measured non-invasively.
6. The method of clause 1, wherein the derivative target parameter(s) are determined from the generic target parameter(s) by the Bolus Induced Response of Deviations (BIRD) mathematical model set forth in the specification and figures.

7. A method for optimizing the state of hydration (optimizing mVLT or OmVLT) of a subject comprising:
a) quantifying the subject's initial baseline generic target parameter(s) of the mathematical BIRD model, which target parameter(s) include but are not limited to parameters such as perfusion index, tissue hemoglobin content, cardiac stroke volume, global end diastolic volume index, end diastolic filling ratio, and markers of plasmadilution such as arterial, venous and capillary hemoglobin concentration, wherein the minimum set of target parameters required is arterial Hb and/or capillary Hb;
b) intravenously administering to the subject an iso-oncotic, iso-osmotic non-cellular liquid at the highest safe rate over a period of 2-5 minutes, wherein the volume infused does not exceed 5 ml/per kg of subject's body weight, and then quantifying the subject's peak generic target parameter(s) immediately after the end of the administering;
c) quantifying the subject's acute residual generic target parameter(s) after a period of 5 minutes from the end of step b) but before 6 minutes from the end of step b) without further intravenous administration of liquid to the subject;
d) determining, by formulae set forth in the specification based on the generic target parameters of steps a), b) and c), derivative target parameter(s) of the mathematic BIRD model set forth in the specification;
e) intravenously administering to the subject an iso-oncotic, iso-osmotic non-cellular liquid at the highest safe rate over a period of 2-5 minutes, and the volume infused does not exceed 5 ml per kg of subject body weight, and then quantifying the subject's peak generic target parameter(s) immediately after the end of the administering;
f) quantifying the subject's acute residual generic target parameters after a period of 5 minutes from the end of step b) but before 6 minutes from the end of step b) without further intravenous administration of liquid to the subject;
g) determining, by formulae set forth in the specification based on the generic target parameter(s) of steps e), f) and g), derivative target parameters of the mathematical BIRD model set forth in the specification;
h) comparing the derivative target parameter(s) determined in step d) and step g) to a derivative-trend-matrix which is (i) FIG. 43B for derivatives defining arteriovenous dilution difference, (ii) FIG. 43C and FIG. 45 for derivatives defining arterial plasmadilution; (iii) FIG. 44-B,C for capillary derivatives to determine the hydration status;
i) iteratively repeating steps e) through h) until the diagnosis of hydration status derived in step h) is normohydration, optihydration, or within the interval between normohydration and optihydration.
8. The method of clause 1, for maximizing the stroke volume, wherein a capillary BIRD trend provides indirect monitoring of the stroke volume response to the mVLT step; and a positive response indicates a further mVLT step is required.
9. The method of clause 8, wherein the positive response is an increase of (a),
(b)_capillary C_cRBD, (c) S_RBD (d) and/or arterial deviations.
10. The method of clause 1 for the continuous diagnosis of hydration status to determine when switching should be administered a volume therapy or a transfusion therapy.
11. A method for determining if a subject is in need of administration of crystalloids or administration of a blood volume expanding agent comprising performing the method of clause 1, wherein when an overhydration status is determined in step i) no further of administration of crystalloids for the maximization of stroke volume is required and it is determined that the subject is in need of administration of a volume expander.
12. The method of clause 1 or 6, wherein the iso-oncotic, iso-osmotic non-cellular liquid is acetated Ringer's solution.
13. The method of clause 2, wherein the target parameter is hemoglobin concentration and is the subject's venous, arteriolar, or capillary hemoglobin concentration.
14. The method of clause 1, wherein the target parameter is hemoglobin concentration and is the capillary hemoglobin concentration.
15. The method of clause 1, 2, 3, 4, or 13-14, wherein the hemoglobin concentration is measured non-invasively.
16. The method of clause 2 or 8, wherein the derivatives of stroke volume are determined from the hemoglobin concentration.
17. The method of clause 1, wherein in step f) the peak generic target parameters are measured just after the end of the administering and wherein in step g) acute residual generic target parameters are measured just after the 5 minutes of the end of step f).
18. A device comprising:
   a) a non-invasive blood hemoglobin concentration sensor attached to a computing apparatus so as to provide blood hemoglobin concentration input to the computing apparatus;
   b) the computing apparatus;
   c) an intravenous fluid pump controller which is attached to, and controlled by output from, the computing apparatus,
   wherein the output is related to the input by the BIRD algorithm set forth in the specification and figures.
19. A device comprising:
   a) a non-invasive blood hemoglobin concentration sensor attached to a computing apparatus comprising a memory so as to provide blood hemoglobin concentration input to the computing apparatus;
   b) the computing apparatus comprising the memory, which memory is communicatively coupled to one or more processors, the memory comprising at least one sequence of instructions which when executed by the processor causes the processor to perform the determination steps and comparison steps of clause 1 or 7 so as to provide an output to an intravenous fluid pump controller;
   an intravenous fluid pump controller which is attached to, and controlled by output from, the computing apparatus.
20. The device of clause 18 or 19, wherein the non-invasive blood hemoglobin concentration sensor provides a real-time blood hemoglobin measurement input.
21. The device of clause 18 or 19 wherein the intravenous fluid pump controller controls an infusion or transfusion pump, wherein the infusion or transfusion is being provided to a subject from whom the blood hemoglobin concentration is measured as the input in part a).
22. A non-transitory computer-readable storage medium with an executable program stored thereon, wherein the program instructs a microprocessor to perform the steps a) through i) of clause 1 or a) through i) of clause 7.
23. The non-transitory computer-readable storage medium of clause 22, wherein the steps of intravenous administration are performed by sending a signal to a device so as to effect intravenous delivery of the liquid by the device to the subject.

### REFERENCES

1. Lobo D N, Allison S P. Fluid, electrolyte and nutrient replacement. In: Burnand K G, Young A E, Lucas J, Rowlands B J, Scholefield J, eds. The New Aird's Companion in Surgical Studies. 3rd ed. London: Churchill Livingstone, 2005: 20-41.
2. Kehlet H, Wilmore D W: Evidence-based surgical care and the evolution of fast track surgery. Ann Surg 248:189, 2008.
3. Bundgaard-Nielsen M, Ruhnau B, Secher N H, Kehlet H. Flow-related techniques for preoperative goal-directed fluid optimization. British Journal of Anaesthesia 2007; 98(1):38-44.
4. Gan T J, Soppitt A, Maroof M, el-Moalem H, Robertson K M, Moretti E, Dwane D, Glass PS: Goal-directed intraoperative fluid administration reduces length of hospital stay after major surgery. Anesthesiology 2002; 97:820-6.
5. Andrijauskas A. New algorithm for the perioperative goal directed fluid management and related haemodynamic monitoring in non-cardiac patients. In: Proceedings of 2nd World Congress of Total Intravenous Anaesthesia. Berlin (Germany), Apr. 23-25, 2009. pp 97-115. Available from: www.medimond.com/proceedings/moreinfo/120090423.htm
6. Jacob M, Chappell D, Rehm M. Clinical update: perioperative fluid management. The Lancet 2007; 369:1984-1986.
7. Goddard N G, Menadue L T, Wakeling H G. A case for routine oesophageal Doppler fluid monitoring during major surgery becoming a standard of care. Br J Anaesth 2007; 99:599.
8. Holte K, Kehlet H. Fluid therapy and surgical outcomes in elective surgery: a need for reassessment in fast-track surgery. J Am Coll Surg 2006; 202 (6):971-989.
9. Pearse R, Dawson D, Fawcett J, Rhodes A, Grounds R M, Bennett ED. Early goal-directed therapy after major surgery reduces complications and duration of hospital stay. A randomised, controlled trial [ISRCTN38797445]. Crit Care 2005; 9:R687-93.
10. Wakeling H G, McFall M R, Jenkins C S, et al. Intraoperative oesophageal Doppler guided fluid management shortens postoperative hospital stay after major bowel surgery. Br J Anaesth 2005; 95:634-42.
11. Noblett S E, Snowden C P, Shenton B K, Horgan A F. Randomized clinical trial assessing the effect of Doppler-optimized fluid management on outcome after elective colorectal resection. Br J Surg; 2006; 93:1069-76.
12. Rex S, Brose S, Metzelder S, et al. Prediction of fluid responsiveness in patients during cardiac surgery. Br J Anaesth 2004; 93:782-8.
13. Svensen C. H., Olsson J., Hahn R. G., Intravascular fluid administration and hemodynamic performance during open abdominal surgery, Anesth. Analg., 2006, 103, 671-676.
14. Lopes M R, Auler J O C, Michard F. Volume management in critically ill patients: new insights. Clinics. 2006; 61(4):345-50.
15. Lobo D N, Allison S P. Fluid, electrolyte and nutrient replacement. In: Burnand K G, Young A E, Lucas J, Rowlands B J, Scholefield J, eds. The New Aird's Companion in Surgical Studies. 3rd ed. London: Churchill Livingstone, 2005: 20-41.
16. Lahner D, Kabon B, Marschalek C, Chiari A, Pestel G, Kaider A, Fleischmann E, Hetz H. Evaluation of stroke volume variation obtained by arterial pulse contour analysis to predict fluid responsiveness intraoperatively. Br. J. Anaesth. 2009 103: 346-351.
17. Hofer C K, Senn A, Weibel L, Zollinger A. Assessment of stroke volume variation for prediction of fluid responsiveness using the modified FloTrac and PiCCOplus system. Crit Care 2008; 12: R82.
18. Berridge J C, Warring-Davies K. Bland J M, Quinn A C. A new, minimally invasive technique for measuring cardiac index: clinical comparison of continuous cardiac dynamic monitoring and pulmonary artery catheter methods. Anaesthesia 2009; 64:961-967.
19. Boulepaep E L. Organisation of the cardiovascular system. In: Baron W F, Boulepaep E L, eds. Medical physiology. Philadelphia: Saunders; 2003.p. 423-446.
20. Boulepaep E L. Microcirculation. In: Baron W F, Boulepaep E L, eds. Medical physiology. Philadelphia: Saunders; 2003.p. 463-482.
21. Gelman S. Venous function and central venous pressure. Anesthesiology 2008; 108:735-48.
22. Mark P. M. Harms, Johannes J. van Lieshout, Morten Jenstrup, Frank Pott, Niels H. Secher Postural effects on cardiac output and mixed venous oxygen saturation in humans. Experimental Physiology (2003) 88.5, 611-616.
23. Magder S, Scharf S M: Venous return, Respiratory-Circulatory Interactions in Health and Disease. Edited by Scharf S M, Pinsky M R, Magder S. New York, Marcel Dekker, 2001, pp 93-112.
24. Gregory Ray. Plasma Dilution Special Situations. Presentation in: Physician's Council AATB 2009 Spring Meeting Mar. 28, 2009 Orlando, Fla.
25. Stahle L, Nilsson A, Hahn R G. Modelling the volume of expandable body fluid spaces during i.v. fluid therapy. Br J Anaesth 1997; 78: 138-43.
26. Holte K, Foss N B, Svensen C, Lund C, Madsen J L, Kehlet H. Epidural anesthesia, hypotension, and changes in intravascular volume. Anesthesiology 2004; 100: 281-6.
27. Andrijauskas A. Homeostatic blood states theory. Thesis. Vilnius University. Vilnius, Lithuania 2006. Also available from: www.dissertation.com/book.php?method=ISBN&book=1599426536
28. Andrijauskas A. Blood volume homeostasis: normal versus physiologic target volume. Transfusion Alternatives in Transfusion Medicine (TATM) 2007; 9, Suppl.1: 39-41. Also available from: www3.interscience.wiley.com/journal/118489799/issue
29. Brandstrup B. Fluid therapy for the surgical patient. Best Pract Res Clin Anaesthesiol 2006; 20:265-83. Grocott M P, Mythen M G, Gan T J: Perioperative fluid management and clinical outcomes in adults. Anesth Analg 2005; 100:1093-106.
30. Stevens T, Garcia J G, Shasby D M, Bhattacharya J, Malik A B: Mechanisms regulating endothelial cell barrier function. Am J Physiol Lung Cell Mol Physiol 2000; 279:L419-22.
31. Starling E. On the absorption of fluid from the connective tissue spaces. J Physiol (Lond) 1896; 19:312-26.
32. Jacob M, Bruegger D, Rehm M, Stoeckelhuber M, Welsch U, Conzen P, Becker B F. The endothelial glycocalyx affords compatibility of Starling's principle and high cardiac interstitial albumin levels. Cardiovasc Res 2007; 73:575-86.
33. Adamson R H, Lenz J F, Zhang X, Adamson G N, Weinbaum S, Curry F E. Oncotic pressures opposing filtration across non-fenestrated rat microvessels. J Physiol 2004; 557:889-907.
34. Luft J H. Fine structures of capillary and endocapillary layer as revealed by ruthenium red. Fed Proc 1966; 25:1773-83.
35. Rehm M, Zahler S, Lotsch M, Welsch U, Conzen P, Jacob M, Becker BF. Endothelial glycocalyx as an additional barrier determining extravasation of 6% hydroxyethyl starch or 5% albumin solutions in the coronary vascular bed. Anesthesiology 2004; 100:1211-23.
36. Vink H, Constantinescu A A, Spaan J A. Oxidized lipoproteins degrade the endothelial surface layer. Implications for platelet-endothelial cell adhesion. Circulation 2000; 101:1500-2.
37. Pries A R, Secomb T W, Gaehtgens P. The endothelial surface layer. Pflugers Arch 2000; 440:653-66.
38. Hu X, Weinbaum S. A new view of Starling's hypothesis at the microstructural level. Microvasc Res 1999; 58:281-304. Hu X, Adamson R H, Liu B, Curry F E, Weinbaum S. Starling forces that oppose filtration after tissue oncotic pressure is increased. Am J Physiol Heart Circ Physiol 2000; 279:H1724-36.
39. Levick J R. Revision of the Starling principle: New views of tissue fluid balance. J Physiol 2004; 557:704.
40. Chappell D, JacobM, Hofmann-Kiefer K, Conzen P, Rehm M. A rational approach to perioperative fluid management. Anesthesiology 2008; 109:723-40.
41. Kohl B A, Deutschman C S. The inflammatory response to surgery and trauma. Curr Opin Crit Care 2006; 12:325-32.
42. Kamp-Jensen M, Olesen K L, Bach V, Schutten H J, Engquist A. Changes in Serum electrolyte and atrial natriuretic peptide concentrations, acid-base and haemodynamic status after rapid infusion of isotonic saline and Ringer lactate solution in healthy volunteers. Br J Anaesth 1990; 64:606-10.
43. Flint M H. Some observations on the vascular supply of the nail bed and terminal segments of the finger. British Journal of Plastic Surgery 1956; 8:186-195.
44. Svensen C, Hahn R G. Volume kinetics of Ringer solution, dextran 70, and hypertonic saline in male volunteers. Anesthesiology 1997; 87:204-12.
45. Drobin D, Hahn R G. Volume kinetics of Ringer's solution in hypovolemic volunteers. Anesthesiology 1999; 90:81-91.
46. Brauer K I, Svense'n C, Hahn R G, Traber L, Prough DS. Volume kinetic analysis of the distribution of 0.9% saline in conscious versus isoflurane-anesthetized sheep. Anesthesiology 2002; 96:442-9.
47. Drobin D, Hahn R G. Kinetics of isotonic and hypertonic plasma expanders. Anesthesiology 2002; 96:1371-80
48. Ewaldsson C A, Hahn R G. Kinetics and extravascular retention of acetated Ringer s solution during isoflurane and propofol anesthesia for thyroid surgery. Anesthesiology 2005; 103:460-9.
49. Svensen C H, Rodhe P M, Olsson J, Borsheim E, Aarsland A, hahn R G. Arteriovenous differences in plasma dilution and the distribution kinetics of lactated Ringer's solution. Anesth Analg 2009; 108:128-33.
50. Hahn R G, Andrijauskas A, Drobin D, Svensen C, Ivaskevicius J. A volume loading test for the detection of dehydration. Medicina. 2008; 44:953-9.
51. A. Andrijauskas. New method of tracing blood hemoglobin concentration to hematocrit ratio for monitoring plasma dilution and osmotic changes in blood. Transfusion Alternatives in Transfusion Medicine (TATM) 2007; 9, Suppl.1: 36-38. Also available from: www3.interscience.wiley.com/journal/118489799/issue
52. Andrijauskas A, Ivaskevicius J. New method of tracing blood hemoglobin concentration to hematocrit ratio for monitoring plasma dilution and osmotic origin shifts in blood. Medicina (Kaunas) 2006; 42(3):181-186.
53. McMurdy J W, Jay G D, Suner S, Crawford G. Noninvasive optical, electrical, and acoustic methods of total hemoglobin determination. Clinical Chemistry 2008; 54(2):264-272.
54. Current news from Masimo. Available from:
   www.masimo.com/news/index.cfm#01252007
55. Mokken F C, et al. Differences in peripheral arterial and venous hemorheologic parameters. Ann Hematol 1996; 73:135-137.
56. Yang Z W, et al. Comparison of blood counts in venous, fingertip, and arterial blood and their measurement variation. Clin. Lab. Haem. 2001; 23:155-159.
57. Gomez-Simon A, et al. Evaluation of four rapid methods for hemoglobin screening of whole blood donors in mobile collection settings. Transfusion and Apheresis Science. 2007; 36:235-242.
58. Bland J M, et al. Statistical methods for assessing agreement between two methods of clinical measurement. Lancet 1986; 1:307-310.
59. Wiig H, Rubin K, Reed R K. New active role of the interstitium in control of interstitial fluid pressure: potential therapeutic consequences. Acta Anaesthesiologica Scandinavica 2003; 47(2):111-121.
60. Lamhout L et al. Comparison of the accuracy of noninvasive hemoglobin monitoring by spectrophotometry (SpHb) and HemoCue° with automated laboratory hemoglobin measurement. Anesthesiology 2011; 115:548-54.
61. Cannesson M et al. Does the pleth variability index indicate the respiratory-induced variation in the plethysmogram and arterial pressure waveforms? Anesth Analg 2008; 106:1189-94.

TABLE 1
Assesment and monitoring of fluid balance

| Parameter | Significance |
|---|---|
| History | Alerts to likelihood of fluid deficit (e.g. vomiting/diarrhoea/haemorrhage) or |
| | excess (e.g. from intraoperative fluids) |
| Weighing | 24-h change in weight (performed under similar conditions)-best measure |
| | of change in water balance. Simple to carry out by bedside. |
| Fluid balance | Inherent inaccuracies in measurement and recording. Does not measure |
| charts | insensible loss. Large cumulative error over several days. Good measure |
| | of changes in urine output, fistula loss, gastric aspirate, etc. |
| Urine output | <30 ml/h is commonly used as indication for fluid infusion, but in the |
| | absence of other features of intravascular hypovolaemia is usually due to |
| | the normal oliguric response to surgery. Urine quality (e.g. urine:plasma |
| | urea or osmolality ratio) is just as important, particularly in the complicated |
| | patient. |
| Blood | Cuff measurements may not always correlate with intra-arterial monitoring. |
| pressure | Does not necessarily correlate with flow. Affected by drugs, etc. |
| | Nonetheless, a fall is compatible with intravascular hypovolaemia, |
| | particularly when it correlates with other parameters such as pulse rate, |
| | urine output, etc. |
| Capillary refill | Slow refill compatible with, but not diagnostic of volume deficit. Can be |
| | influenced by temperature and peripheral vascular disease. |
| Autonomic | Pallor and sweating, particularly when combined with tachycardia, |
| responses | hypotension and oliguria are suggestive of intravascular volume deficit, but |
| | can also be caused by other complications, e.g. pulmonary embolus or |
| | myocardial infarction. |
| Skin turgor | Diminished in salt and water depletion, but also caused by ageing, cold and |
| | wasting. |
| Dry mouth | Usually due to mouth breathing, but compatible with salt and water |
| | depletion. |
| Sunken facies | May be due to starvation or wasting from disease, but compatible with salt |
| | and water depletion. |
| Serum | Indicates ratio of electrolytes to water in the extracellular fluid and is a poor |
| biochemistry | indicator of whole body sodium status. Hyponatraemia most commonly |
| | caused by water excess. If change in water balance over 24 h is known, |
| | then change in serum sodium concentration can guide sodium balance. |
| | Hypokalaemia nearly always indicates the need for potassium |
| | supplementation. Blood bicarbonate and chloride concentrations measured |
| | on point of care blood gas machines are useful in patients with acid-base |
| | problems including iatrogenic hyperchloraemia. |
| Urine | Urine sodium concentration reflects renal perfusion and a low value (<20 mmol/L) |
| biochemistry | indicates renal hypoperfusion. Measurement of urine sodium |
| | allows assessment of postoperative sodium mobilisation (see text) Urine |
| | potassium measurement is helpful in assessing the cause of refractory |
| | hypokalaemia. Urine urea excretion increases several fold in catabolic |
| | states (e.g. sepsis) and is an indication for provision of additional free water |
| | to avoid hypernatraemia and uraemia. |

TABLE 2
Association of midcapillary hydraulic pressure and
related arteriolar and venular tone

| | | Arteriolar dilation | Arteriolar constriction |
|---|---|---|---|
| | Resting tone | Venular constriction | Venular dilation |
| Arterioles | 60 | 60 | 60 |
| Capillaries | 25 | 40 | 20 |
| Venulae | 15 | 15 | 15 |

TABLE 3

| | | | | | Data point | | | | | | | |
|---|---|---|---|---|---|---|---|---|---|---|---|---|
| | | | | | | mVLT | | | Description and use of the derivative variable | | | |
| Generic variable(s) | | Derivative variable | | | Initial | step | | Equation # in | Mathematical | Physiological | Meaning/use in the | |
| Definition | Abbreviation | Definition | Abbreviation | Refference | baseline | 1 | >1 | BIRD-math | decription | definition/meaning | mVLT method | Deficiency |
| Hemoglobin | Hb | Plasma | PD | Total | A | A | A | 1-3 | Fractional change of Hb | Total plasma dilution at | Close to nill or negative in | to |
| concentration | | dilution | | dilution | | | | | at the end of mVLT step | intial baseline and | the dilution plateau part of | spotting the |
| | | | | | | | | | in respect to intial | following 5 min steady | the hydration plateau. | plateau. |
| | | | | | | | | | baseline Hb value. | state after each test bolus. | | |
| Plasma dilution | PD | Plasma | PDD | Response | NA | A | A | 4 | Shift of total plasma | Plasma dilution efficacy of | Positive during the transitory | Non-specific. |
| | | dilution | | | | | | | dilution during a single | a single mVLT step. | initial rehydration and | |
| | | difference | | | | | | | mVLT step. | | overhydration. | |
| | | | | | | | | | | | Negative or close to nill at | End-dilution |
| | | | | | | | | | | | transitory maximized | plateau is |
| | | | | | | | | | | | normal interstitial | usually |
| | | | | | | | | | | | hydration (dilution plateau). | overriden. |
| Plasma dilution | PDD | Mean | MPD | Response | NA | NA | A | 5 | Mean shift of plasma | Tendency of plasma | Decreases within the | Non-specific. |
| difference | | plasma- | | variability | | | | | dilution in two | dilution efficacy in two | hydration plateau, and | |
| | | dilution | | | | | | | consecutive mVLT | consecutive mVLT steps. | increases at its exit. | |
| | | difference | | | | | | | steps. | | | |
| Plasma dilution | PDD | Variation of | VPR | Response | NA | NA | A | 6 | The difference between | Deviation of plasma | Negative turning into | Insufficiently |
| difference and | and | plasma- | | variation | | | | | the shift of total plasma | dilution efficacy from its | positive is the marker of | informative for |
| mean plasma | MPD | dilution | | | | | | | dilution in the last mVLT | tendency in two | ‘exit’ from hydration | the evaluation |
| dilution | | response | | | | | | | step and the mean of | consecutive steps. | plateau of the single | of multiple |
| difference | | | | | | | | | two last steps. | | capillary-lymphatic bed. | plateaus. |
| | | | | | | | | | | | Comparison of maximal | Needs |
| | | | | | | | | | | | positive values between two | selection of |
| | | | | | | | | | | | ‘exits’ from the hydration | maximal VPR |
| | | | | | | | | | | | plateau; increase means the | values |
| | | | | | | | | | | | shift towards transitory | between two |
| | | | | | | | | | | | maximization of interstitial | ‘exits’ from |
| | | | | | | | | | | | hydration of the whole body. | plateaus. |
| Variation of | VPR | Absolute | ABS- | Responsiveness | NA | NA | A | 7 | Absolute VPR value. | The overall excitability of | Supportive information: | Needs support |
| plasma-dilution | | variation of | VPR | | | | | | | plasma dilution. | increasing ABS-VPR is a | from the |
| response | | plasma- | | | | | | | | | marker of the shift towards | evaluation of |
| | | dilution | | | | | | | | | transitory maximization of | VPR (as |
| | | response | | | | | | | | | interstitial hydration. | above). |

Generic—Measured parameter or previously calculated derivative used to calculate new derivatives.
Derivative—Non-measurable variable that was derived by equations of the BIRD-math model.
mVLT step—Minimal volume loading test step.
Hb—Hemoglobin concentration (aHb-arterial and vHb-venous).
A—Available.
NA—Not applicable.

1. A method for determining the baseline state of the whole-body interstitial hydration (diagnostic mVLT or DmVLT) of a subject comprising:
a) quantifying the subject's initial baseline generic target parameters, wherein the derivative target parameter(s) are determined from the generic target parameter(s) by the Bolus Induced Response of Deviations (BIRD) mathematical model set forth in the specification and figures, wherein the target parameter(s) are chosen from the group of arterial, venous and capillary hemoglobin concentration ([Hb]), or wherein at least arterial or capillary Hb;
b) intravenously administering to the subject an iso-oncotic, iso-osmotic non-cellular liquid at the highest safe rate over a period of 2-5 minutes, wherein the total volume administered is 1.5 to 2.5 ml per kg of the subject's lean body mass;
c) quantifying the subject's generic target parameter(s) after a period of 5 minutes from the end of step b), but before 6 minutes from the end of step b) without further intravenous administration of a liquid to the subject;
d) determining by formulae the derivative target parameter(s) for the respective generic target parameter(s) obtained in steps a), and c);
e) intravenously administering to the subject an iso-oncotic, iso-osmotic non-cellular liquid at the highest safe rate over a period of 2-5 minutes, wherein the total volume administered is 1.5 to 2.5 ml per kg of the subject's lean body mass;
f) quantifying the subject's acute residual generic target parameter(s) after a period of 5 minutes from the end of step b), but before 6 minutes from the end of step e), without further administration of liquid to the subject;
g) determining by formulae the derivative target parameter(s) of the respective generic target parameter(s) obtained in steps e) and g);
h) comparing the derivative target parameter(s) determined in step d) and step g) to a specific pattern(s) of the diagnostic criteria set forth in the specification; and
i) iteratively repeating steps e) through h) until the diagnosis of baseline interstitial hydration status is derived by fitting the dynamics of the derivative target parameter(s) to a specific pattern(s) of the diagnostic criteria set forth in the specification.

2. The method of claim 1 wherein the target parameter(s) are chosen from the group of arterial, venous and capillary hemoglobin concentration ([Hb]), or wherein at least arterial or capillary Hb.

3. The method of claim 1 wherein the target parameter is arterial [Hb] and/or capillary [Hb].

4. The method of claim 3, wherein the target parameters are arterial [Hb] and capillary [Hb].

5. The method of claim 3 wherein the capillary hemoglobin concentrations are measured non-invasively.

6. The method of claim 1, wherein the derivative target parameter(s) are determined from the generic target parameter(s) by the Bolus Induced Response of Deviations (BIRD) mathematical model set forth in the specification and figures.

7. A method for optimizing the state of the whole-body interstitial hydration (optimizing mVLT or OmVLT) of a subject comprising:
a) quantifying the subject's initial baseline generic target parameters, wherein the derivative target parameter(s) are determined from the generic target parameter(s) by the Bolus Induced Response of Deviations (BIRD) mathematical model set forth in the specification and figures, wherein the target parameter(s) are chosen from the group of arterial, venous and capillary hemoglobin concentration ([Hb]), or wherein at least arterial or capillary Hb;
b) intravenously administering to the subject an iso-oncotic, iso-osmotic non-cellular liquid at the highest safe rate over a period of 2-5 minutes, wherein the total volume administered is 1.5 to 2.5 ml per kg of the subject's lean body mass;
c) quantifying the subject's generic target parameter(s) after a period of 5 minutes from the end of step b), but before 6 minutes from the end of step b) without further intravenous administration of a liquid to the subject;
d) determining by formulae the derivative target parameter(s) for the respective generic target parameter(s) obtained in steps a) and c);
e) intravenously administering to the subject an iso-oncotic, iso-osmotic non-cellular liquid at the highest safe rate over a period of 2-5 minutes, wherein the total volume administered is 1.5 to 2.5 ml per kg of the subject's lean body mass;
f) quantifying the subject's acute residual generic target parameter(s) after a period of 5 minutes from the end of step b), but before 6 minutes from the end of step e), without further administration of liquid to the subject;
g) determining by formulae the derivative target parameter(s) of the respective generic target parameter(s) obtained in steps e), f) and g);
h) comparing the derivative target parameter(s) determined in step d) and step g) to a specific pattern(s) of the diagnostic criteria set forth in the specification; and
j) iteratively repeating steps e) through h) until the operator preferred transitory whole body interstitial hydration state is derived by fitting the dynamics of the derivative target parameter(s) to a specific pattern(s) of the diagnostic criteria set forth in the specification.

8. The method of claim 1, for maximizing the cardiac stroke volume, wherein a plasma dilution variation, and especially capillary trend, provides indirect monitoring of the stroke volume response to the mVLT step.

9. The method of claim 8, wherein the positive cardiac stroke volume response (its increase) is a decrease of capillary plasmadilution response variation (VPR).

10. The method of claim 1 for the continuous diagnosis of hydration status to determine when switching to a volume therapy or a transfusion therapy should be administered.

11. The method of claim 1, wherein the iso-oncotic, iso-osmotic non-cellular liquid is preferably acetated Ringer's solution, but the other iso-osmotic crystalloid solutions can be deployed.

12. The method of claim 2, wherein the target parameter is hemoglobin concentration and is the subject's venous, arteriolar, or capillary hemoglobin concentration.

13. The method of claim 1, wherein the target parameter is hemoglobin concentration and is the capillary hemoglobin concentration.

14. The method of claim 1, wherein the hemoglobin concentration is measured non-invasively.

15. The method of claim 2, wherein the derivatives of stroke volume are determined from the hemoglobin concentration.

16. The method of claim 1, wherein in step g) acute residual generic target parameters are measured just after the 5 minutes of the end of step f).

17. A device comprising:
d) a non-invasive blood hemoglobin concentration sensor attached to a computing apparatus so as to provide blood hemoglobin concentration input to the computing apparatus;
e) the computing apparatus;
f) an intravenous fluid pump controller which is attached to, and controlled by output from, the computing apparatus, wherein the output is related to the input by the BIRD algorithm set forth in the specification and figures or
a device comprising:
c) a non-invasive blood hemoglobin concentration sensor attached to a computing apparatus comprising a memory so as to provide blood hemoglobin concentration input to the computing apparatus;
d) the computing apparatus comprising the which memory is communicatively coupled to one or more processors, the memory comprising at least one sequence of instructions which when executed by the processor causes the processor to perform the determination steps and comparison steps of claim 1 so as to provide an output to an intravenous fluid pump controller; an intravenous fluid pump controller which is attached to, and controlled by output from, the computing apparatus.

18-26. (canceled)
